(19) United States
(12) Patent Application Publication
(10) Pub. No.: US 2024/0376049 A1
(43) Pub. Date: Nov. 14, 2024

(54) AZACARBENE CARBON FREE RADICAL MOLECULES, MIXTURES AND FORMULATIONS COMPRISING SAME, AND USES THEREOF IN ORGANIC ELECTRONIC DEVICES

(71) Applicant: Zhejiang Brilliant Optoelectronic Technology Co.,Ltd., Taizhou (CN)

(72) Inventors: Chao SHI, Taizhou (CN); Hong HUANG, Taizhou (CN); Junyou PAN, Taizhou (CN)

(73) Assignee: Zhejiang Brilliant Optoelectronic Technology Co.,Ltd., Taizhou (CN)

(21) Appl. No.: 18/783,969

(22) Filed: Jul. 25, 2024

Related U.S. Application Data
(63) Continuation of application No. PCT/CN2023/071163, filed on Jan. 9, 2023.

(30) Foreign Application Priority Data
Jan. 27, 2022 (CN) 202210100314.3

(51) Int. Cl. C07D 207/06; C07D 401/04; C07D 403/04; C07D 413/10; C07D 417/10; C07D 487/04; C07D 498/04; C07D 498/16; C07D 513/04; C07F 5/02; H10K 85/60

(52) U.S. Cl. CPC C07D 207/06; C07D 401/04; C07D 403/04; C07D 413/10; C07D 417/10; C07D 487/04; C07D 498/04; C07D 498/16; C07D 513/04; C07F 5/027; H10K 85/622; H10K 85/633; H10K 85/654; H10K 85/656; H10K 85/6572; H10K 85/658

(57) ABSTRACT

Disclosed are azacarbene carbon free radical molecules including a structure of formula (I). T is a single bond, or a carbon atom, or a nitrogen atom, when T is a single bond, Ar³ does not exist. Also provided are formulations containing at least one azacarbene carbon free radical molecule, and at least one organic solvent. Further provided are organic electronic devices containing at least one azacarbene carbon free radical molecule.

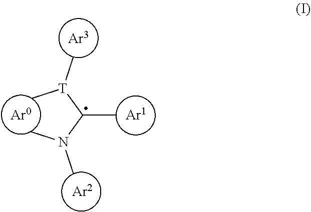

### CROSS-REFERENCE TO RELATED APPLICATIONS

The present application is a continuation of International Application No. PCT/CN2023/071163, filed on Jan. 9, 2023, which claims priority to Chinese Patent Application No. 202210100314.3, filed on Jan. 27, 2022. All of the aforementioned applications are incorporated herein by reference in their entireties.

### TECHNICAL FIELD

The present disclosure relates to the field of organic electronic material and device technology, and in particular to an azacarbene carbon free radical molecule, a formulation, and the applications thereof in organic electronic devices, particularly in organic spintronic devices or organic light-emitting diodes. The present disclosure also relates to an organic electronic device comprising the azacarbene carbon free radical molecule, and in particular to an organic spin electron, a light-emitting element, and the applications thereof in display devices and illumination equipment.

### BACKGROUND

Due to the diversity of the synthesis, the low manufacturing cost, and the excellent optical and electrical properties, the organic light-emitting diodes (OLEDs) show great potential for optoelectronic device applications, such as flat-panel displays and lighting.

Up to now, organic free radical emitting material based on open shell can achieve high external quantum efficiency of the device by utilizing 100% doublet excitons, which is comparable to the conventional phosphorescent metal (iridium, platinum) complex materials. In 2018, China Prof. Feng Li of Jilin University successfully prepared a near-infrared electrophosphorescent device by doping organic emitting materials containing trichlorotriphenylmethyl radical (TTM) units into N,N-dicarbazole biphenyl (CBP) with a maximum external quantum efficiency (EQE) of up to 27%. Moreover, this free-radical emitting material only forms doublet excitons in the luminous region of OLEDs, while the bilinear excitons do not have the spin-forbidden blocking problem during the transition process, which makes the internal quantum efficiency of the device theoretically 100%, and can solve the long-standing triplet exciton utilization problem. In addition, such free-radical emitting material is heavy metal-free, low-cost, and green, which has attracted great interest.

However, most of the currently reported stable free-radical emitting materials contain TTM units, the TTM units will inevitably lose chlorine atoms during the evaporation process, resulting in poor material stability, which is not conducive to high-quality displays, and the spectrum of such free-radical emitting material is difficult to be tuned to the blue-green region.

In addition, organic spintronics is the foundation of next-generation computing and storage technologies, and is still a current research hotspot. Free radical compound is potential key material for organic spintronic devices, so that the high-performance free radical compounds for this purpose are urgently to be developed.

Therefore, novel stable and high-performance free-radical emitting materials are urgently to be developed.

### SUMMARY

In one aspect, the present disclosure provides an azacarbene carbon free radical molecule comprising a structure of formula (I):

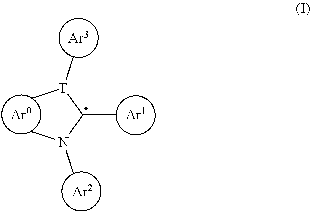

Where T is a single bond, or a carbon atom, or a nitrogen atom, when T is a single bond, Ar³ does not exist; Ar⁰ is a substituted/unsubstituted single bond or double bond, or an alkane, olefin, aromatic hydrocarbon or heteroaromatic cyclic hydrocarbon system, which is unsubstituted or substituted by one or more Rs; Ar¹-Ar³ are each independently selected from an aromatic group containing 6 to 60 ring atoms, a heteroaromatic group containing 5 to 60 ring atoms, or a non-aromatic ring system containing 3 to 30 ring atoms, which are unsubstituted or substituted by R; R, in multiple occurrences, is independently selected from a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, or any combination thereof.

In another aspect, the present disclosure also provides a polymer comprising a repeating unit, the repeating unit comprises at least one structure of formula (I).

In yet another aspect, the present disclosure further provides a mixture comprising at least one azacarbene carbon free radical molecule or polymer as described herein, and at least one other organic functional material, the at least one other organic functional material is selected from a hole-injection material (HIM), a hole-transport material (HTM), an electron-transport material (ETM), an electron-injection material (EIM), an electron-blocking material (EBM), a hole-blocking material (HBM), an emitting material (Emitter), a host material (Host), or an organic dyc.

In yet another aspect, the present disclosure further provides a formulation comprising at least one azacarbene carbon free radical molecule or polymer or mixture as described herein, and at least one organic solvent.

In yet another aspect, the present disclosure further provides an organic electronic device comprising at least one azacarbene carbon free radical molecule or polymer or mixture as described herein.

In addition or alternatively, the organic electronic device as described herein is selected from an organic light-emitting diode (OLED), an organic photovoltaic cell (OPV), an organic light emitting electrochemical cell (OLEEC), an organic field effect transistor (OFET), an organic light emitting field effect transistor, an organic laser, an organic spintronic device, an organic spin-valve, a photodiode, an organic sensor, or an organic plasmon emitting diode (OPED).

Beneficial effects: the present disclosure provides more options for free radical molecular emitting materials by designing and synthesizing novel stable azacarbene carbon free radical molecules; the azacarbene carbon free radical molecule further stabilizes the radical electrons on the carbene by introducing a novel large steric hindrance organic group, thereby improving the luminescence efficiency of the free-radical emitting material and the stability of the free radical molecular material, which provides more material choices for the radical light-emitting device.

### DETAILED DESCRIPTION OF THE EMBODIMENTS

The present disclosure provides an azacarbene carbon free radical molecule, a formulation, and the applications thereof in organic electronic devices, aiming to provide a novel stable and high-performance free radical emitting material. In order to make the objects, the technical solutions and the effects of the present disclosure more clear and definite, the present disclosure is further described in detail below. It should be understood that the embodiments described herein are only intended to explain the present disclosure and are not intended to limit the present disclosure.

As used herein, the term “substituted” means that a hydrogen atom of the compound is substituted.

As used herein, “the number of ring atoms” means that the number of atoms constituting the ring itself of a structural compound (e. g., a monocyclic compound, a fused ring compound, a cross-linked compound, a carbocyclic compound, and a heterocyclic compound) by covalent bonding. When the ring is substituted with a substituent, the atoms contained in the substituent are not included in the ring atoms. The above rule applies for all cases without further specific description. For example, the number of ring atoms of a benzene ring is 6, the number of ring atoms of a naphthalene ring is 10, and the number of ring atoms of a thienyl group is 5.

The term “aromatic group” refers to a hydrocarbon group containing an aromatic ring. The term “heteroaromatic group” refers to an aromatic hydrocarbon group containing at least one heteroatom. The heteroatoms are preferably selected from Si, N, P, O, S and/or Ge, particularly preferably selected from Si, N, P, O and/or S. The term “fused-ring aromatic group” refers to an aromatic group containing two or more rings, in which two carbon atoms are shared by two adjacent rings, i. e., fused rings. The term “fused heterocyclic aromatic group” refers a fused aromatic hydrocarbon group containing at least one heteroatom. For the purposes of the present disclosure, the aromatic groups or heteroaromatic groups comprise not only aromatic ring systems, but also non-aromatic ring systems. Therefore, systems such as pyridine, thiophene, pyrrole, pyrazole, triazole, imidazole, oxazole, oxadiazole, thiazole, tetrazole, pyrazine, pyridazine, pyrimidine, triazine, carbene, and the like is also considered be aromatic groups or heterocyclic aromatic groups for the purposes of this disclosure. For the purposes of the present disclosure, the fused-ring aromatic or fused heterocyclic aromatic ring systems contain not only aromatic or heteroaromatic systems, but also have a plurality of aromatic or heterocyclic aromatic groups linked by short non-aromatic units (<10% of non-H atoms, preferably <5% of non-H atoms, such as C, N or O atoms). Therefore, a system such as 9,9′-spirobifluorene, 9,9-diarylfluorene, triarylamine, diaryl ether, and the like is also considered to be fused-ring aromatic ring systems for the purposes of this disclosure.

In embodiments of the present disclosure, the energy level structure of the organic material, singlet energy level (E_{S1}), doublet energy level (E_{D1}), triplet energy level (E_{T1}), highest occupied molecular orbital (HOMO), and lowest unoccupied molecular orbital (LUMO) play key roles. The determination of these energy levels is introduced as follows.

HOMO and LUMO energy levels can be measured by optoelectronic effect, for example by XPS (X-ray photoelectron spectroscopy), UPS (UV photoelectron spectroscopy), or by cyclic voltammetry (hereinafter referred to as CV). Recently, quantum chemical methods, such as density functional theory (hereinafter referred to as DFT) are becoming effective method for calculating the molecular orbital energy levels.

The singlet energy level E_{S1} of the organic material can be determined by the emission spectrum, and the triplet energy level E_{T1} of the organic material can be measured by low-temperature time-resolved spectroscopy. E_{S1}, E_{D1}, and E_{T1} can also be calculated by quantum simulation (for example, by Time-dependent DFT), for instance with the commercial software Gaussian 09W (Gaussian Inc.), the specific simulation method can be found in WO2011141110. ΔE_{ST} equals E_{S1}-E_{T1}.

It should be noted that the absolute values of HOMO, LUMO, E_{S1}, E_{D1}, and E_{T1} may vary depending on the measurement method or calculation method used. Even for the same method, different ways of evaluation, for example, using either the onset or peak value of a CV curve as reference, may result in different (HOMO/LUMO) values. Therefore, reasonable and meaningful comparison should be carried out by using the same measurement and evaluation methods. In the embodiments of the present disclosure, the values of HOMO, LUMO, E_{S1}, E_{D1}, and E_{T1} are based on the Time-dependent DFT simulation, which however should not exclude the applications of other measurement or calculation methods.

In the present disclosure, (HOMO-1) stands for the energy level of the second highest occupied molecular orbital, (HOMO-2) stands for the energy level of the third highest occupied molecular orbital, and so on. (LUMO+1) stands for the energy level of the second lowest unoccupied molecular orbital, (LUMO+2) stands for the energy level of the third lowest occupied molecular orbital, and so on.

In one aspect, the present disclosure provides an azacarbene carbon free radical molecule comprising a structure of formula (I):

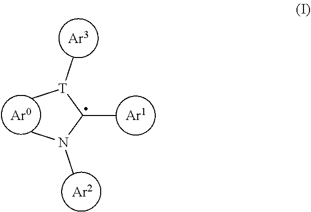

Where T is a single bond, or a carbon atom, or a nitrogen atom, when T is a single bond, Ar³ does not exist; Ar⁰ is a substituted/unsubstituted single bond or double bond, or an alkane, olefin, aromatic hydrocarbon or heteroaromatic cyclic hydrocarbon system, which is unsubstituted or substituted by one or more Rs; Ar¹-Ar³ are each independently selected from an aromatic group containing 6 to 60 ring atoms, a heteroaromatic group containing 5 to 60 ring atoms, or a non-aromatic ring system containing 3 to 30 ring atoms, which are unsubstituted or substituted by R; R is independently selected from a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, or any combination thereof.

In some embodiments, Ar⁰ is selected from a substituted/unsubstituted single bond, a substituted/unsubstituted double bond, a substituted/unsubstituted aromatic group containing 6 to 60 ring atoms, a substituted/unsubstituted heteroaromatic group containing 5 to 60 ring atoms, or a substituted/unsubstituted non-aromatic ring system containing 3 to 30 ring atoms. In some embodiments, Ar⁰ is selected from a substituted/unsubstituted single bond, a substituted/unsubstituted double bond, a substituted/unsubstituted aromatic group containing 6 to 50 ring atoms, a substituted/unsubstituted heteroaromatic group containing 5 to 50 ring atoms, or a substituted/unsubstituted non-aromatic ring system containing 3 to 25 ring atoms. In some embodiments, Ar⁰ is selected from a substituted/unsubstituted single bond, a substituted/unsubstituted double bond, a substituted/unsubstituted aromatic group containing 6 to 40 ring atoms, a substituted/unsubstituted heteroaromatic group containing 5 to 40 ring atoms, or a substituted/unsubstituted non-aromatic ring system containing 3 to 20 ring atoms. In some embodiments, Ar⁰ is selected from a substituted/unsubstituted single bond, a substituted/unsubstituted double bond, a substituted/unsubstituted aromatic group containing 6 to 30 ring atoms, a substituted/unsubstituted heteroaromatic group containing 5 to 30 ring atoms, or a substituted/unsubstituted non-aromatic ring system containing 3 to 15 ring atoms.

In some embodiments, Ar¹-Ar³ are each independently selected from a substituted/unsubstituted aromatic group containing 6 to 30 ring atoms, or a substituted/unsubstituted heteroaromatic group containing 5 to 30 ring atoms; in some embodiments, Ar¹-Ar³ are each independently selected from a substituted/unsubstituted aromatic group containing 6 to 20 ring atoms, or a substituted/unsubstituted heteroaromatic group containing 5 to 20 ring atoms.

In some more preferred embodiments, Ar¹ is selected from a substituted/unsubstituted aromatic group containing 10 to 30 ring atoms, or a substituted/unsubstituted heteroaromatic group containing 8 to 30 ring atoms; in some further preferred embodiments, Ar¹ is selected from a substituted/unsubstituted aromatic group containing 10 to 25 ring atoms, or a substituted/unsubstituted heteroaromatic group containing 8 to 25 ring atoms; in the most further preferred embodiments, Ar¹ is selected from a substituted/unsubstituted aromatic group containing 10 to 20 ring atoms, or a substituted/unsubstituted heteroaromatic group containing 8 to 20 ring atoms.

In some embodiments, Ar⁰ or Ar¹-Ar³ are each independently selected from the following groups or any combination thereof:

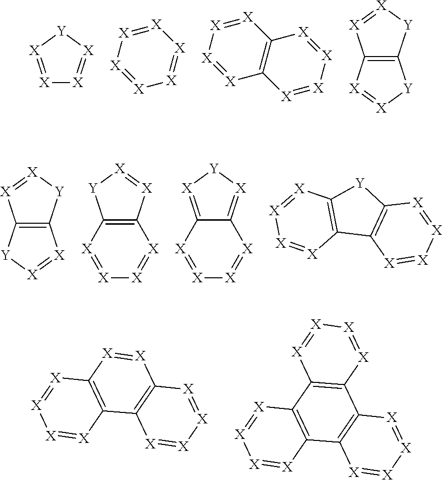

Where Y at each occurrence independently represents CR¹R², NR¹, O, S, SiR¹R², PR¹, P(═O)R¹, S—O, S(═O)₂, or C—O; X at each occurrence independently represents CR¹ or N; each of R¹ and R² at each occurrence is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, or any combination thereof.

Further, Ar⁰ or Ar¹-Ar³ are each independently selected from the following groups or any combination thereof:

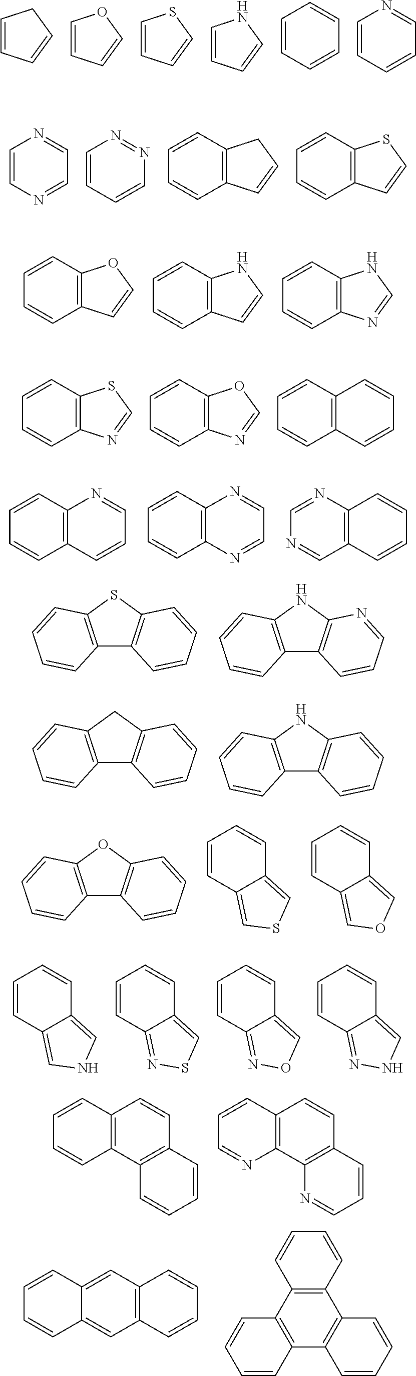

Where H atoms on the ring may be further substituted.

Preferably, in some embodiments, Ar¹-Ar³ are each independently selected from the following groups or any combination thereof:

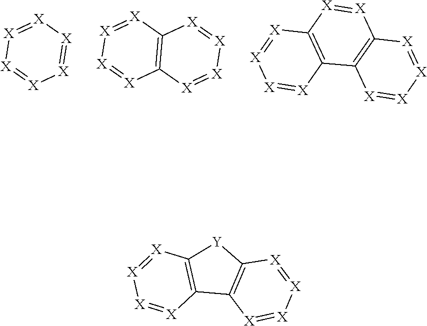

Where X and Y are identically defined as described herein.

In some embodiments, at least one of Ar¹-Ar³ is independently selected from a substituted/unsubstituted fused-ring aromatic group containing 10 to 60 ring atoms or a substituted/unsubstituted fused-ring heteroaromatic group containing 8 to 60 ring atoms; in some embodiments, at least one of Ar¹-Ar³ is independently selected from a substituted/unsubstituted fused-ring aromatic group containing 10 to 50 ring atoms or a substituted/unsubstituted fused-ring heteroaromatic group containing 8 to 50 ring atoms; in some embodiments, at least one of Ar¹-Ar³ is independently selected from a substituted/unsubstituted fused-ring aromatic group containing 10 to 40 ring atoms or a substituted/unsubstituted fused-ring heteroaromatic group containing 8 to 40 ring atoms; in some embodiments, at least one of Ar¹-Ar³ is independently selected from a substituted/unsubstituted fused-ring aromatic group containing 10 to 30 ring atoms or a substituted/unsubstituted fused-ring heteroaromatic group containing 8 to 30 ring atoms; in some embodiments, at least two of Ar¹-Ar³ are independently selected from a substituted/unsubstituted fused-ring aromatic group containing 10 to 30 ring atoms or a substituted/unsubstituted fused-ring heteroaromatic group containing 8 to 30 ring atoms; in some embodiments, Ar¹-Ar³ are each independently selected from a substituted/unsubstituted fused-ring aromatic group containing 10 to 30 ring atoms or a substituted/unsubstituted fused-ring heteroaromatic group containing 8 to 30 ring atoms.

In some embodiments, the fused-ring aromatic group or the fused-ring heteroaromatic group is selected from the following groups or any combination thereof:

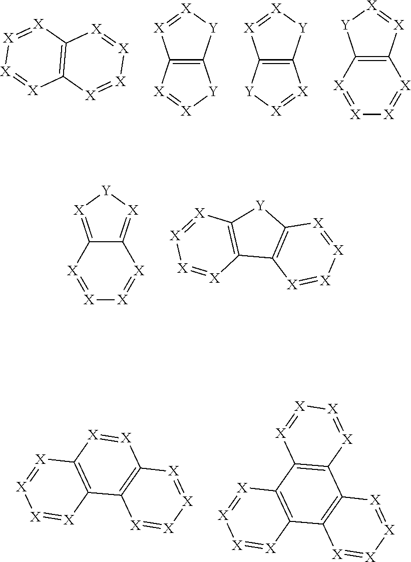

Where X and Y are identically defined as described herein.

More preferably, the fused-ring aromatic group or the fused-ring heteroaromatic group is selected from the group consisting of:

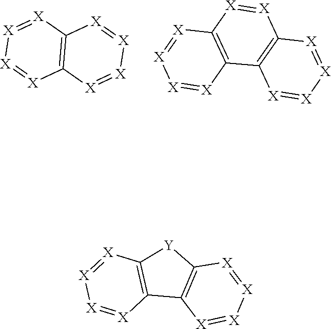

Where X and Y are identically defined as described herein.

In some embodiments, the fused-ring aromatic group is selected from the group consisting of: naphthalene, anthracene, fluoranthene, phenanthrene, triphenylene, perylene, naphthacene, pyrene, benzopyrene, acenaphthylene, fluorene, and derivatives thereof; the fused-ring heteroaromatic group is selected from the group consisting of: benzofuran, benzothiophene, indole, carbazole, pyrroloimidazole, pyrrolopyrrole, thienopyrrole, thienothiophene, furopyrrole, furofuran, thienofuran, benzisoxazole, benzoisothiazole, benzimidazole, quinoline, isoquinoline, o-diazanaphthalene, quinoxaline, phenanthridine, primidine, quinazoline, quinazolinone, and derivatives thereof.

In some embodiments, each T is independently selected from a single bond, a carbon atom, or a nitrogen atom, when Tis a single bond, Ar³ does not exist. Furthermore, the azacarbene carbon free radical molecule is selected from one of formulas (II-1)-(II-5):

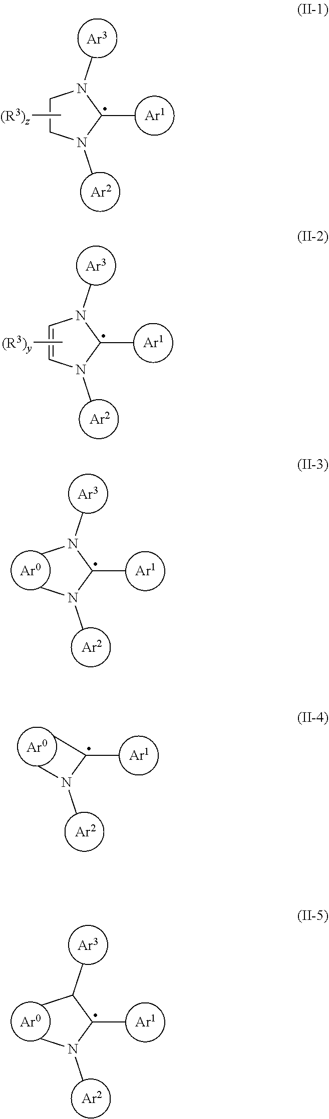

Where Ar⁰ and Ar¹-Ar³ are identically defined as described herein; y is an integer from 0 to 2, z is an integer from 0 to 4; each R³ is identically defined as the above-mentioned R.

In some embodiments, Ar¹ of the azacarbene carbon free radical molecule is independently selected from an unsubstituted or one or more substituted aromatic hydrocarbon or a heteroaromatic cyclic hydrocarbon system, in multiple occurrences may be independently and preferably selected from any one of formulas A1-A20.

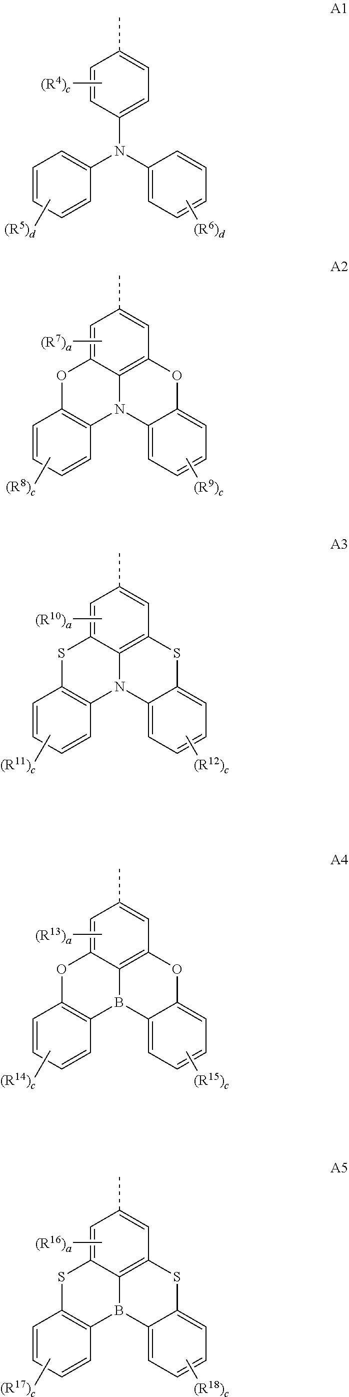

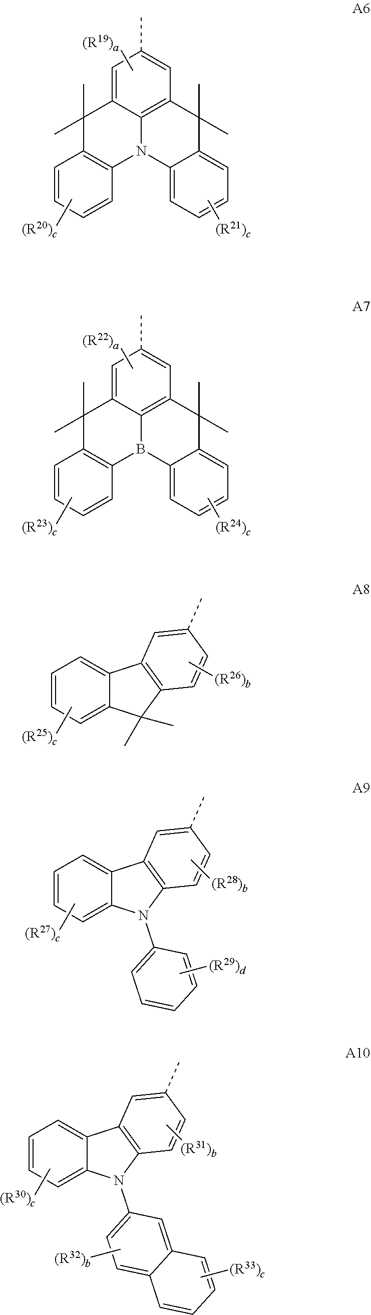

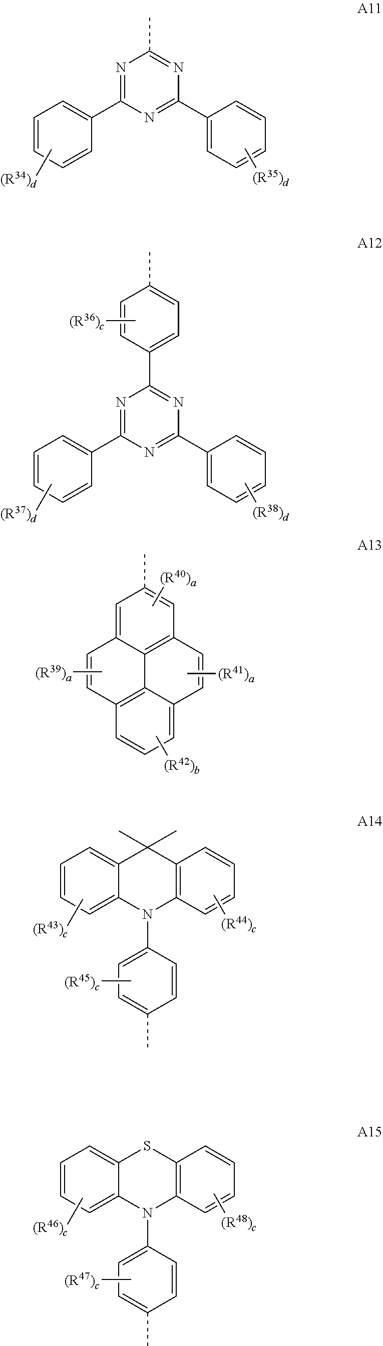

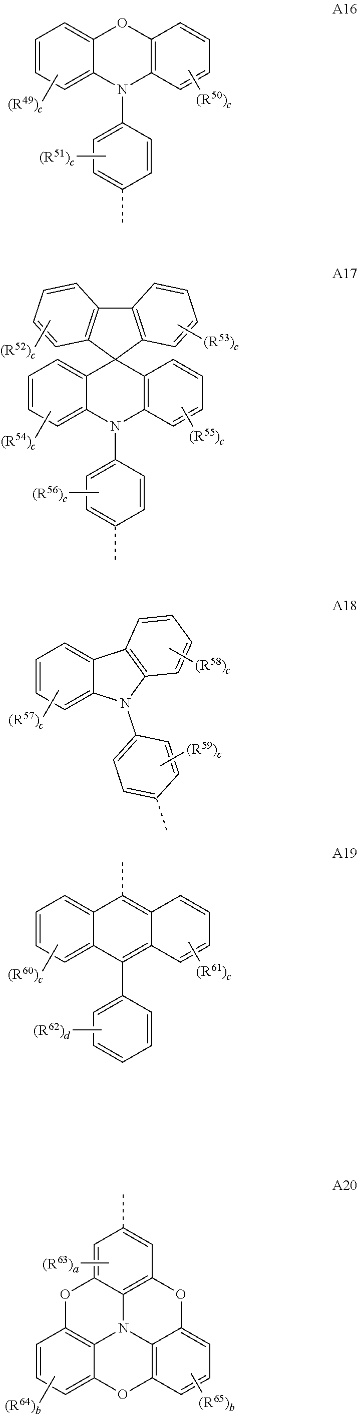

Where a is an integer from 0 to 2; b is an integer from 0 to 3; c is an integer from 0 to 4; d is an integer from 0 to 5; R⁴-R⁶⁵ are each independently selected from F, Cl, Br, I, D, CN, NO₂, CF₃, B(OR₀)₂, Si(R₀)₃, a linear alkane, an alkane ether, a C₁-C₁₀ alkane sulfide, a C₁-C₁₀ branched alkane, a C₁-C₁₀ cycloalkane, a C₃-C₁₀ alkane ether, a C₃-C₁₀ alkane sulfide group, or a C₆-C₁₀ aryl group; each dotted line independently represents a bond directly linked to an azacarbene carbon free radical group.

The term “small molecule” herein refers to a molecule that is no one of following: a polymer, an oligomer, a dendrimer, or a blend. In particular, there are no repeating structures in the small molecule. The molecular weight of the small molecule ≤4000 g/mol, preferably ≤3000 g/mol, and most preferably ≤2000 g/mol.

The term “polymer” comprises homopolymer, copolymer, and block copolymer. Also in the present disclosure, the term “polymer” comprises dendrimer. For the synthesis and application of the dendrimers please refer to [Dendrimers and Dendrons, Wiley-VCH Verlag Gmbh & Co. KGaA, 2002, Ed. George R. Newkome, Charles N. Moorefield, Fritz Vogtle.].

The term “conjugated polymer” refers to a polymer with backbone mainly comprising sp² hybrid orbitals of carbon atoms, well-known examples are polyacetylene and poly(phenylene vinylene). The carbon atoms on the backbones can also be substituted with other non-carbon atoms. Moreover, the above-mentioned structure should still be considered as a conjugated polymer when the sp² hybridization on the backbone is interrupted by natural defects. Also in the present disclosure, the backbone of conjugated polymer comprises aryl amines, aryl phosphines and other heteroarmotics, organometallic complexes, etc.

In some embodiments, the azacarbene carbon free radical molecule is partially deuterated; preferably 10% or more of total H, more preferably 20% or more of total H, further preferably 30% or more of total H, and most preferably 40% or more of total H, are deuterated.

The azacarbene carbon free radical molecule can be used as a functional material in electronic devices, especially in OLED devices. The functional material is selected from a hole-injection material (HIM), a hole-transport material (HTM), an electron-transport material (ETM), an electron-injection material (EIM), an electron-blocking material (EBM), a hole-blocking material (HBM), an emitting material (Emitter), a host material (Host), or an organic dye. In some embodiments, the azacarbene carbon free radical molecule can be used as an emitting material, or an electron-transport material, or a hole-transport material.

Specific examples of the suitable azacarbene carbon free radical molecules are as follows, but not limited thereto.

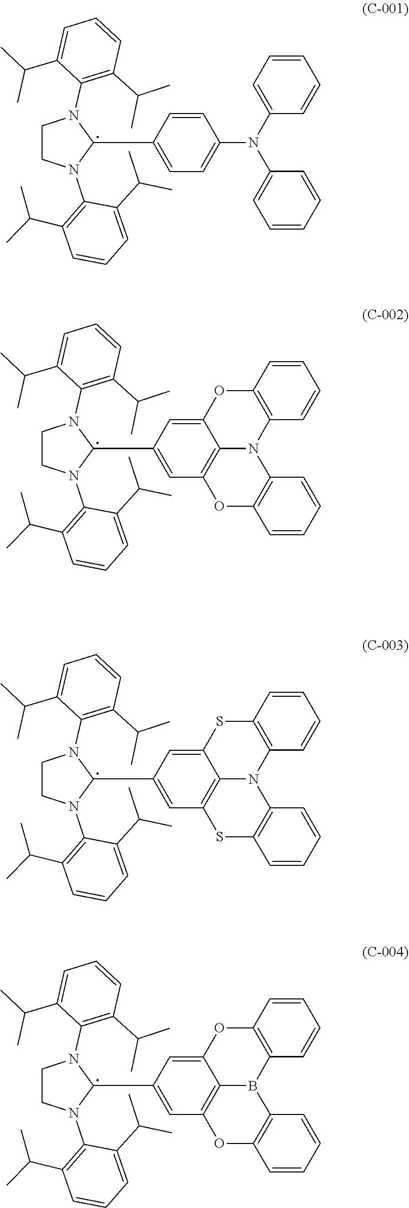

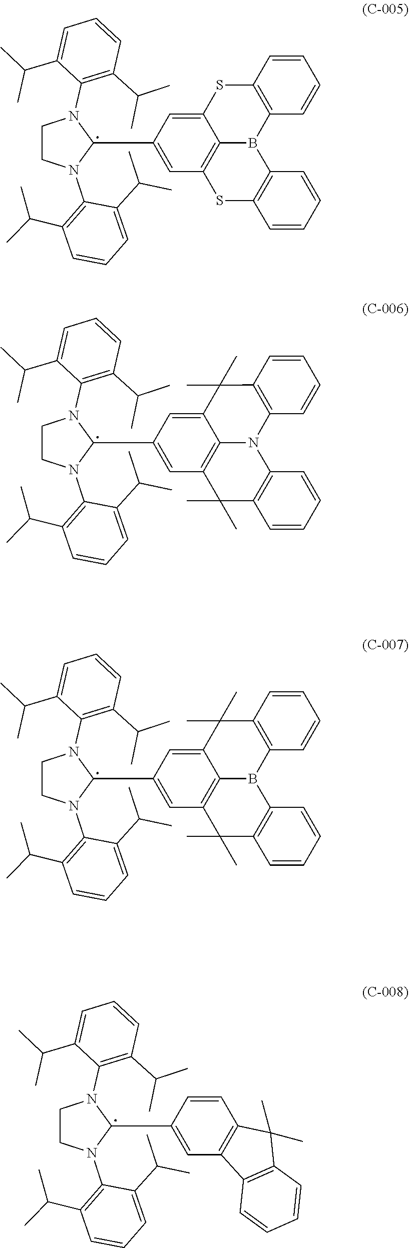

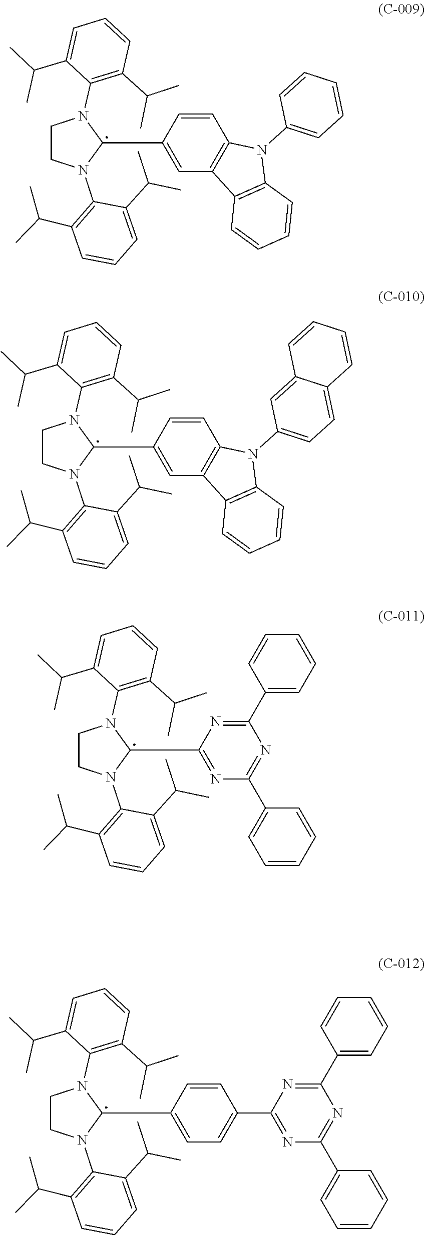

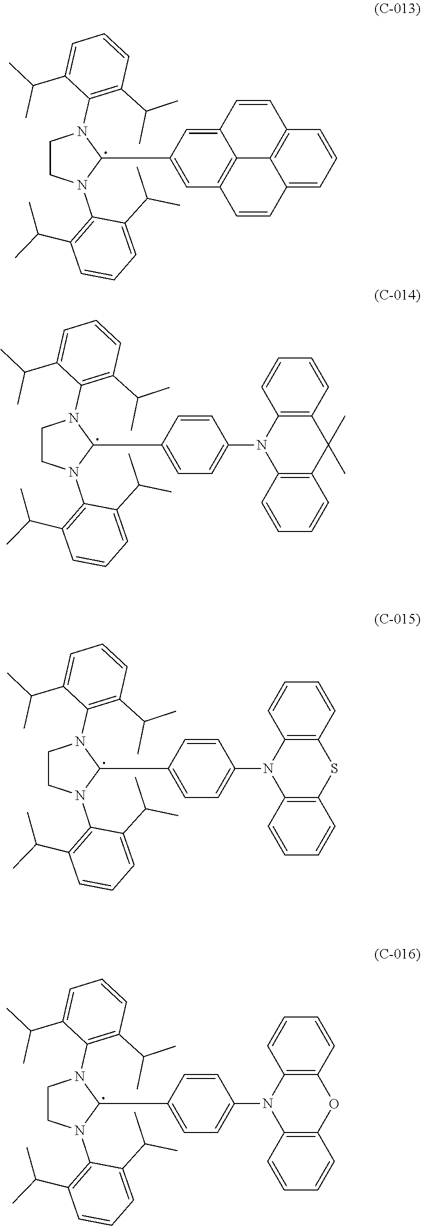

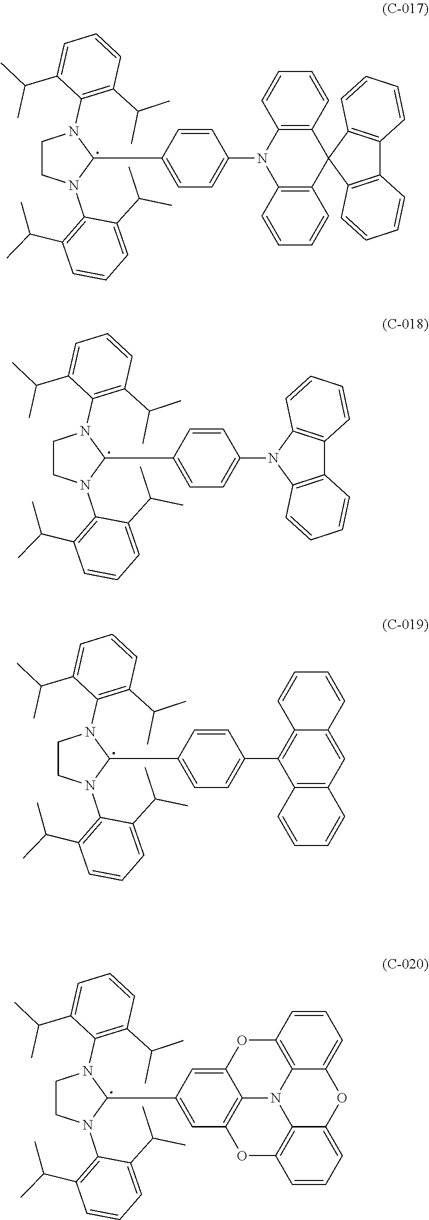

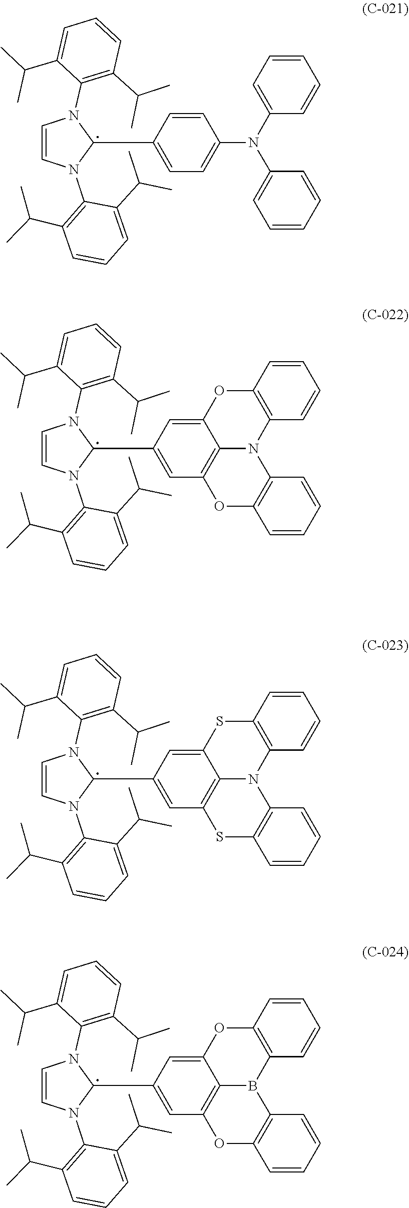

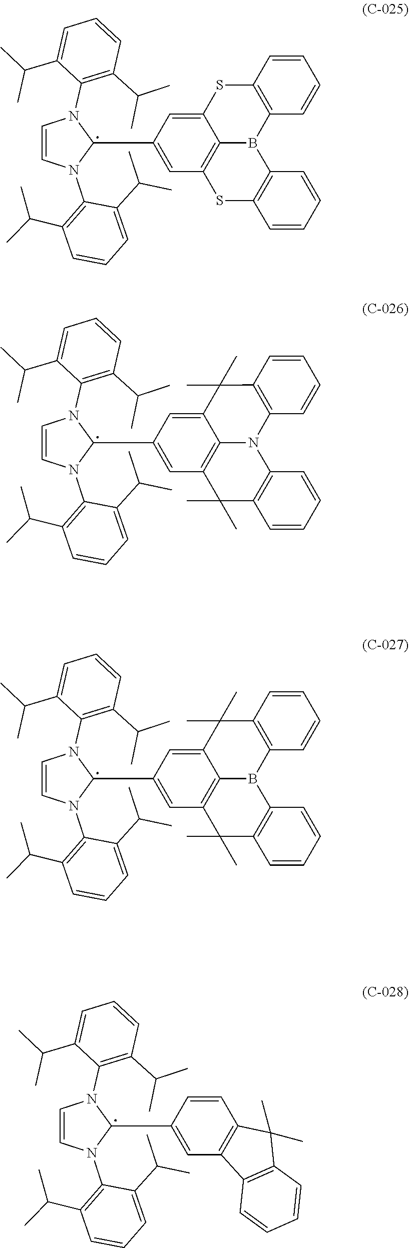

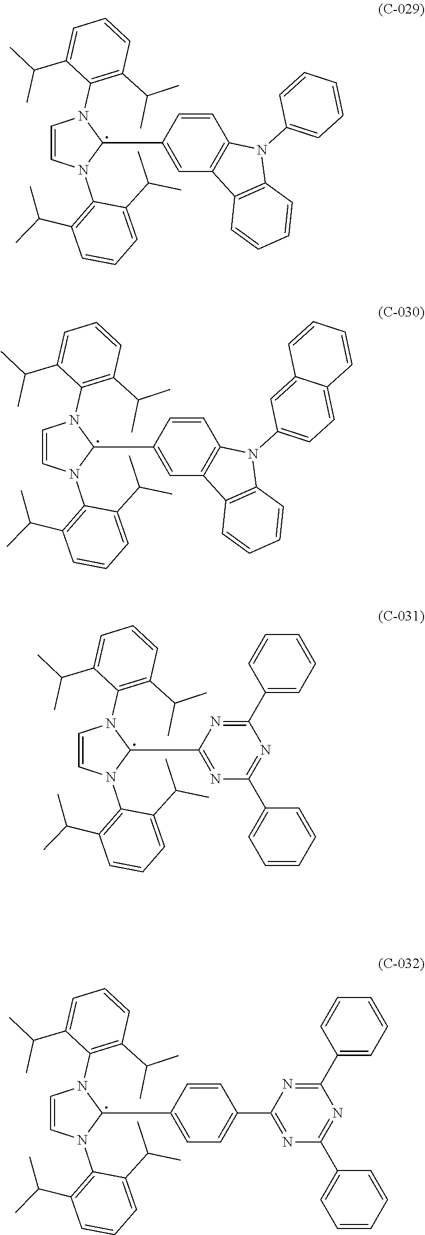

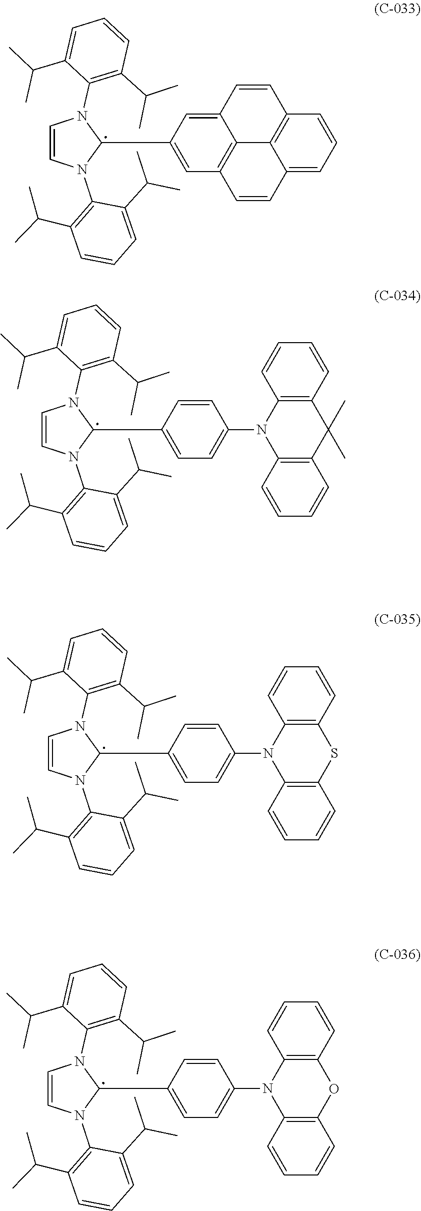

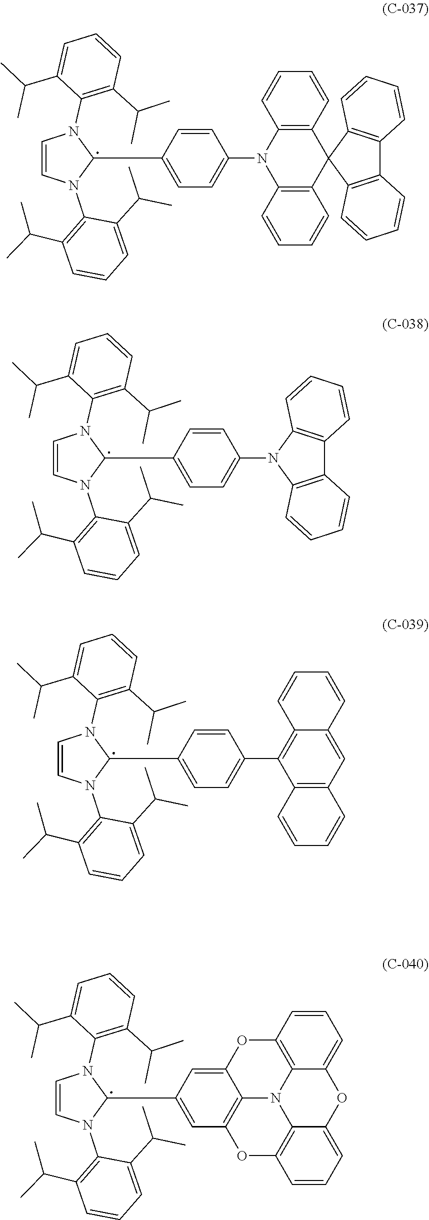

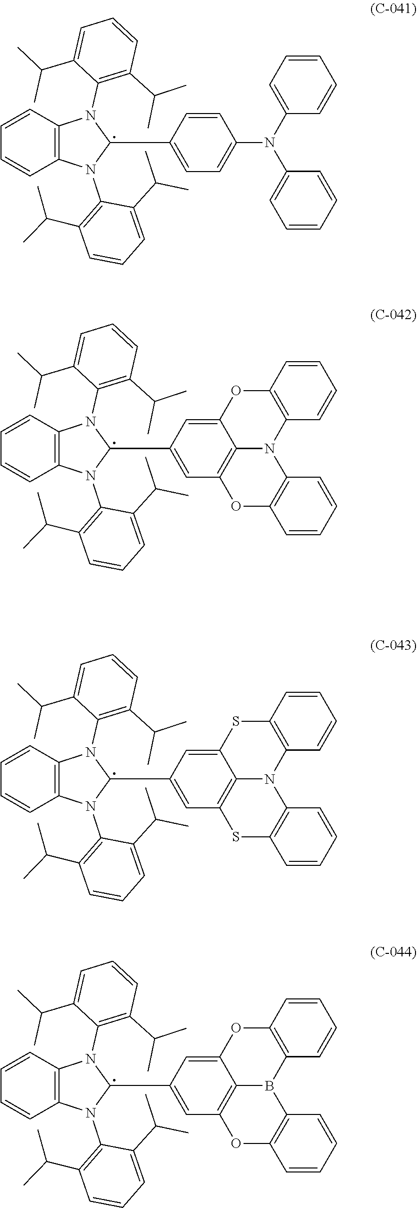

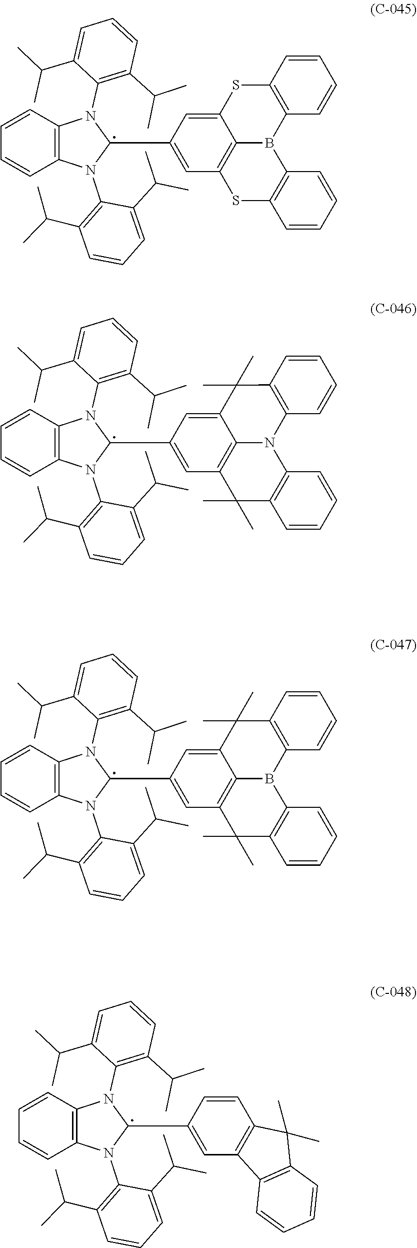

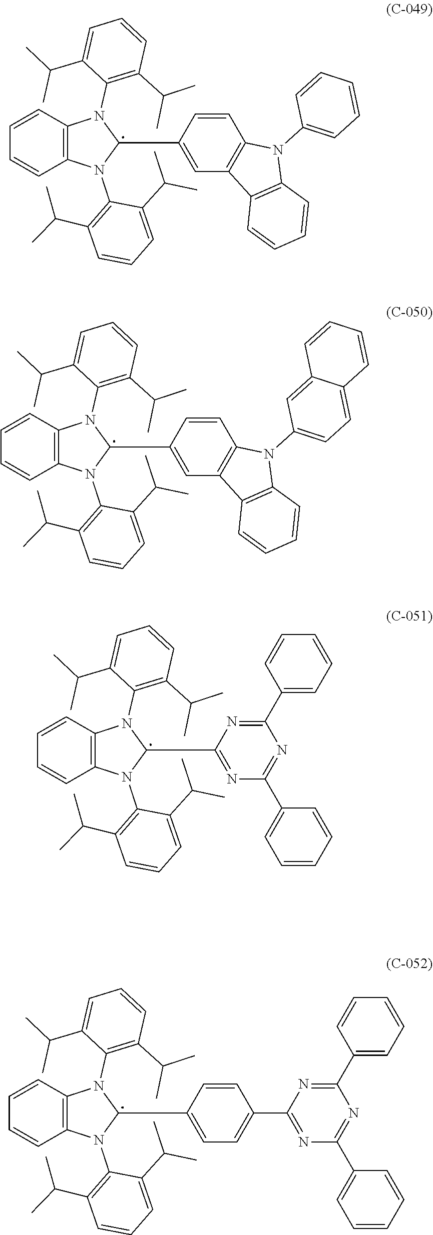

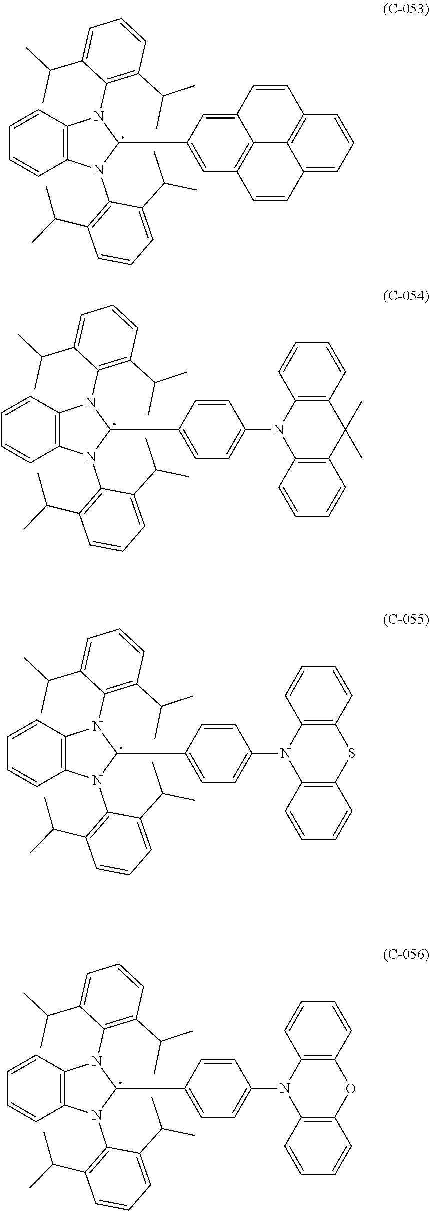

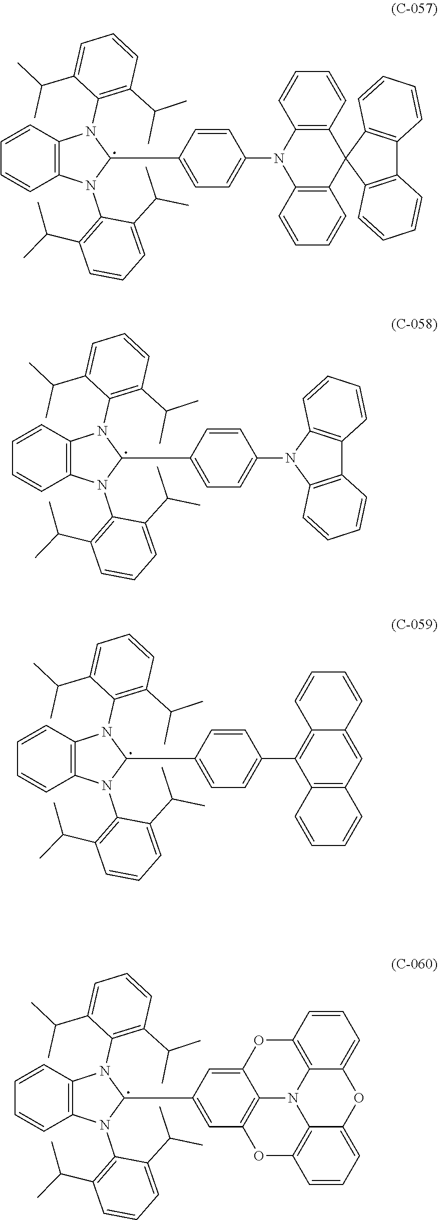

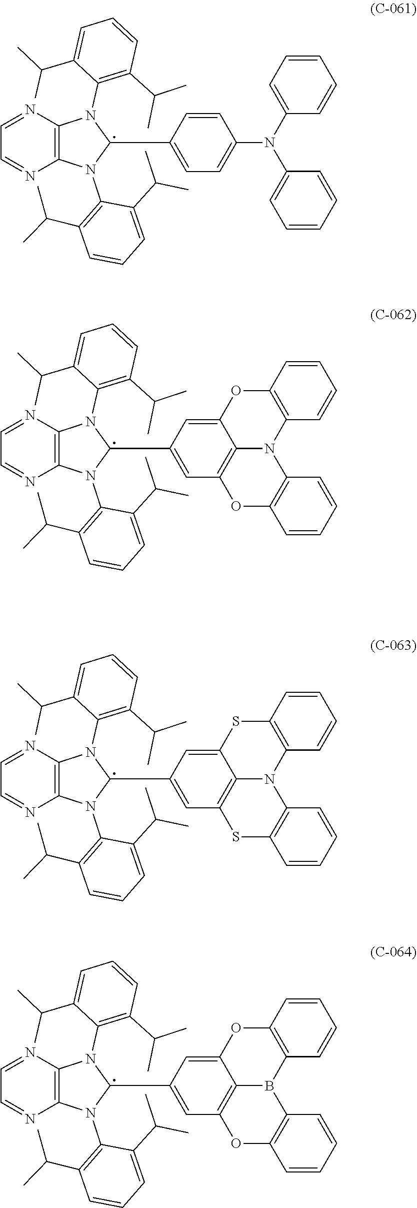

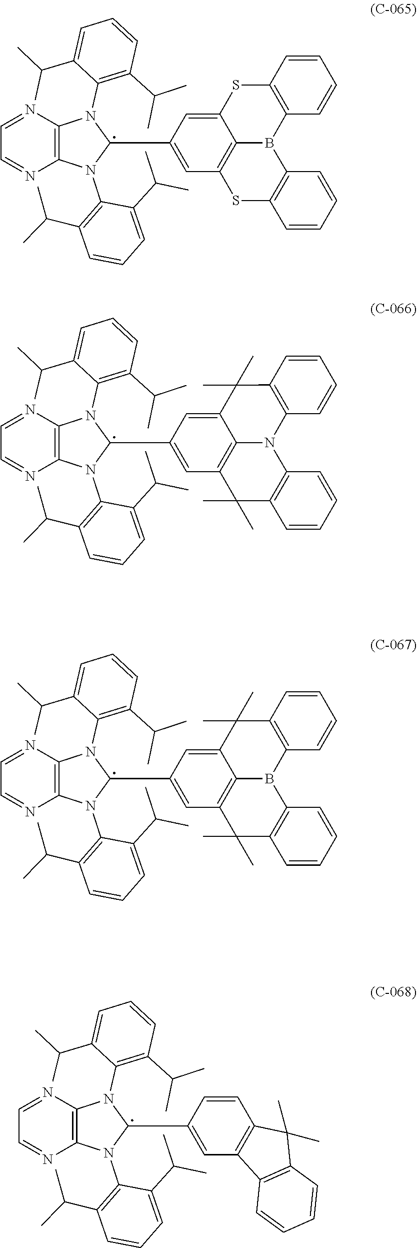

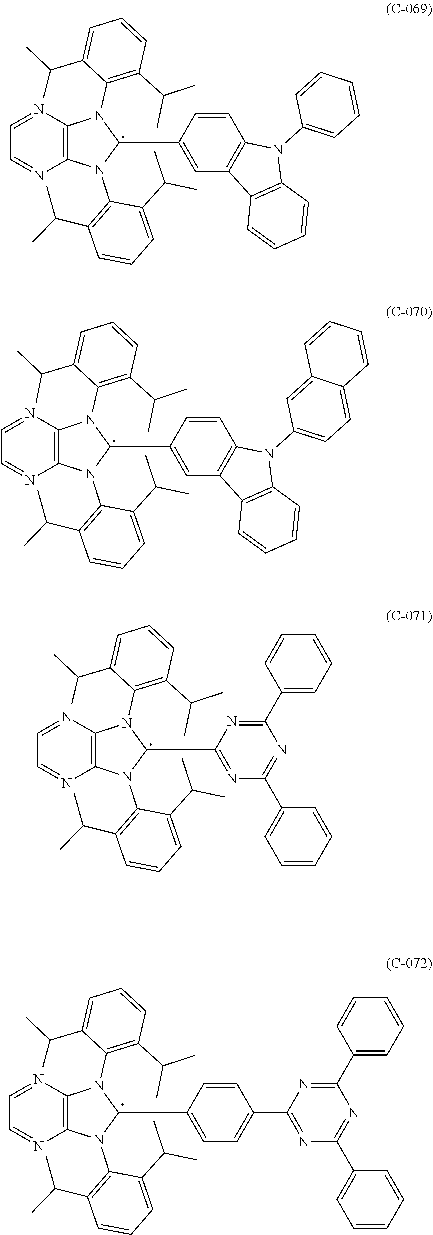

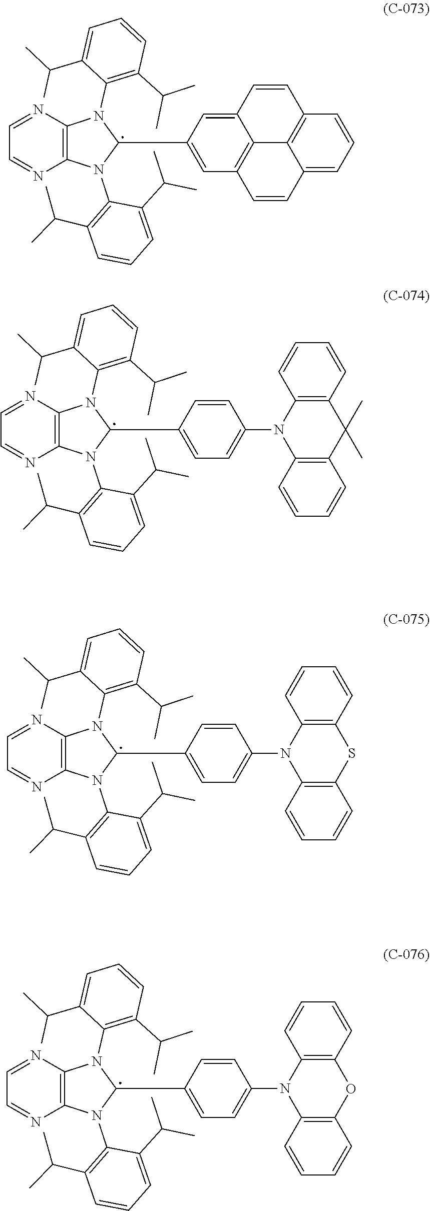

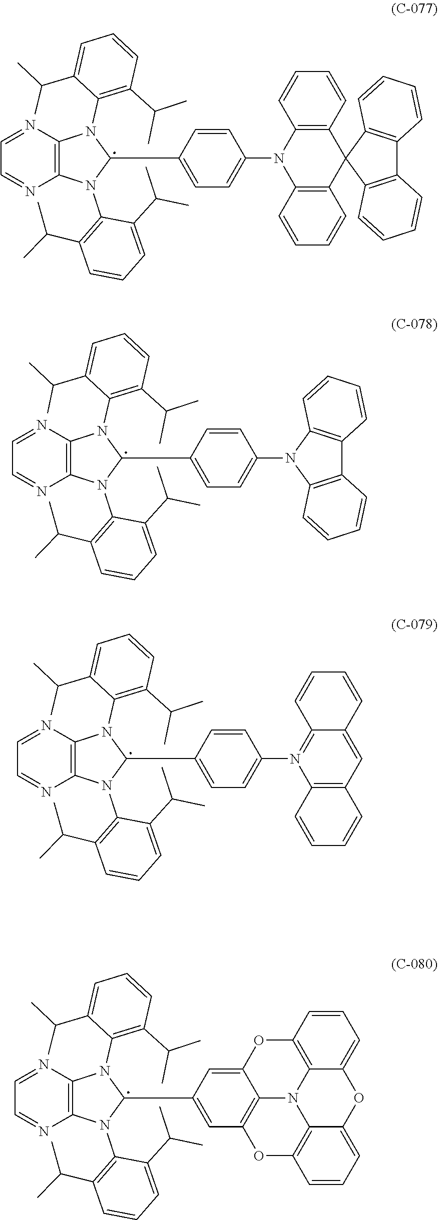

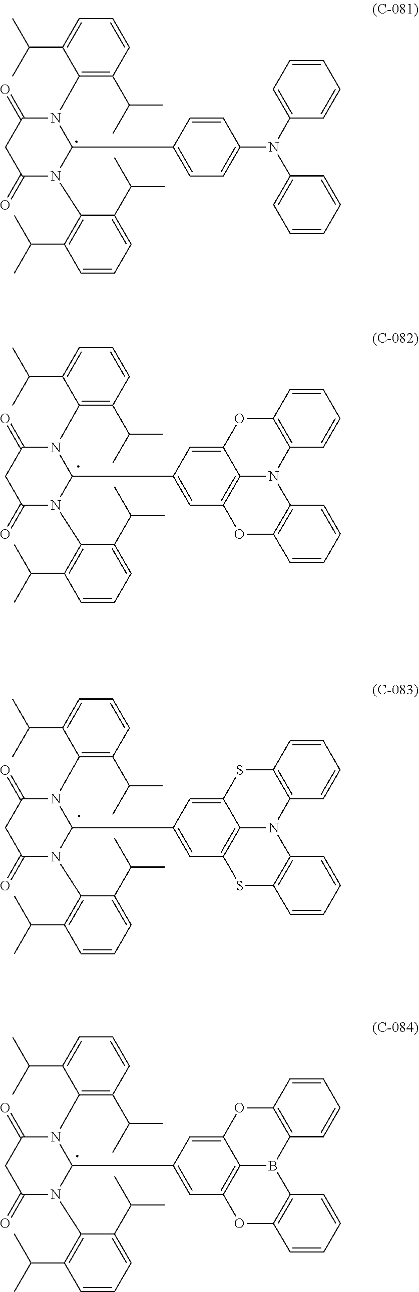

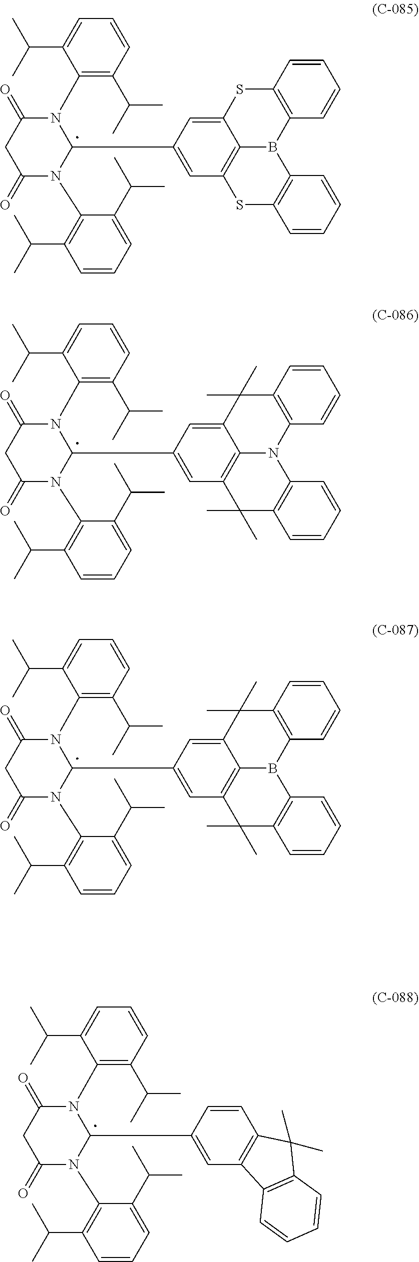

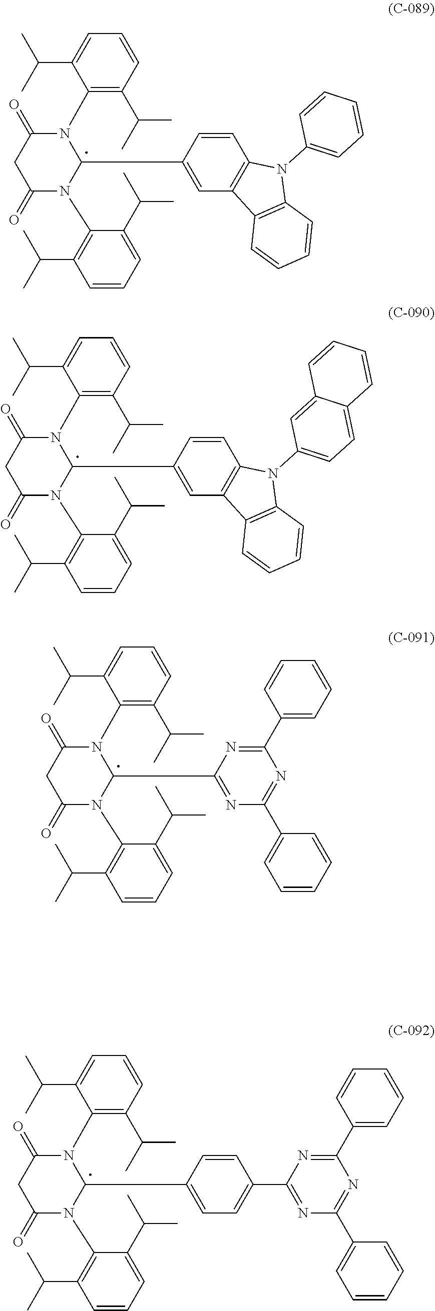

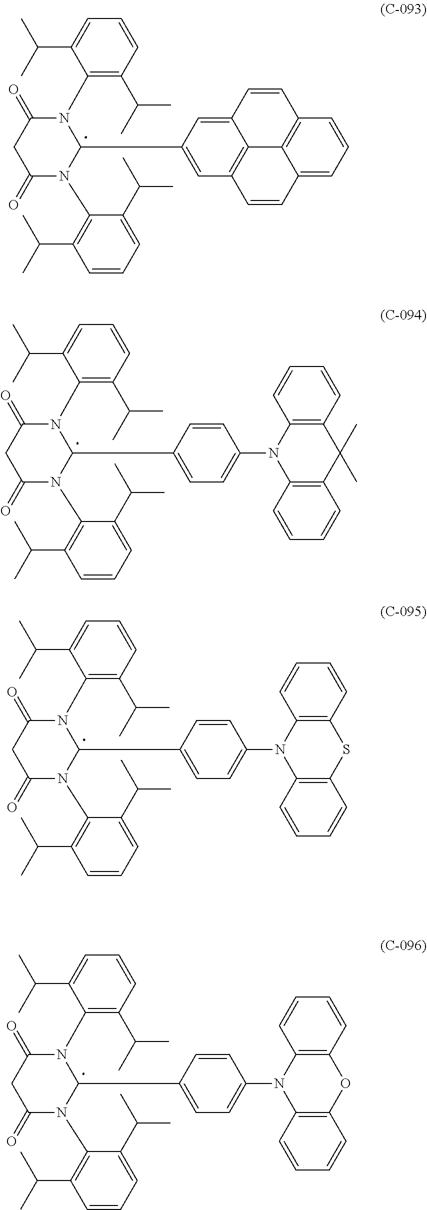

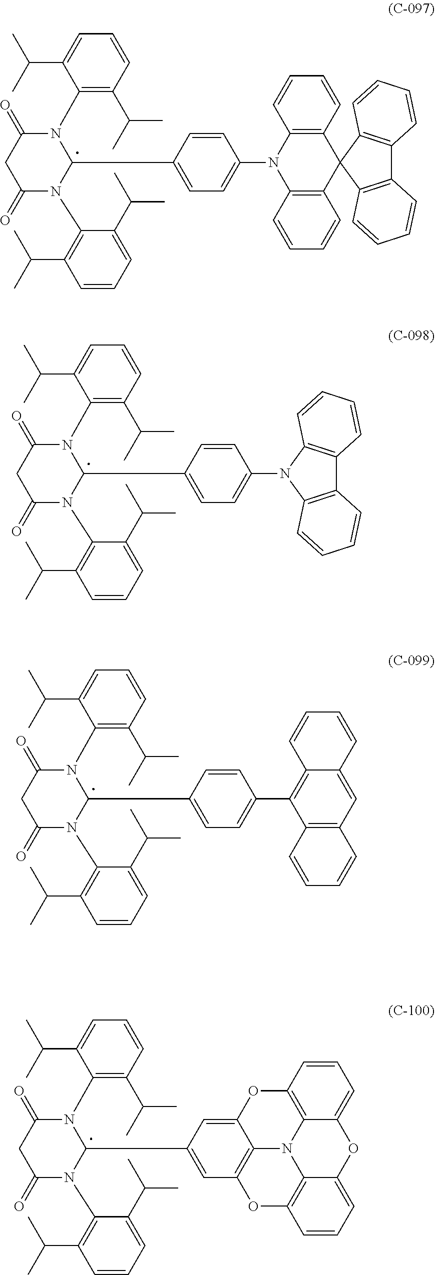

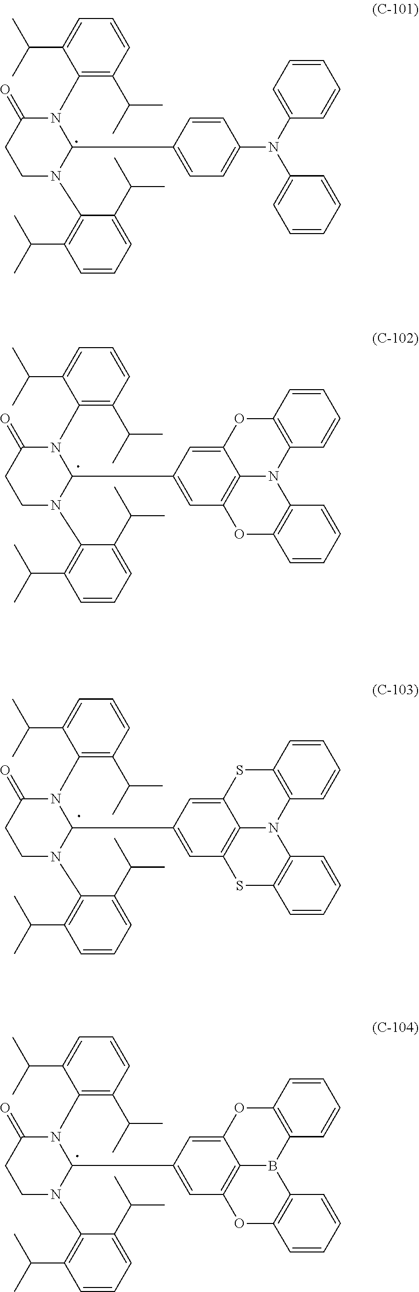

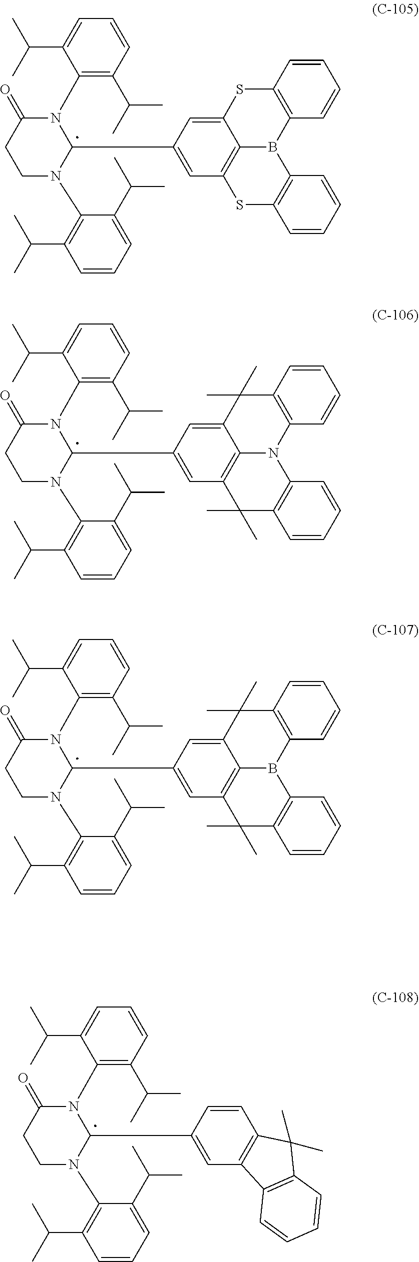

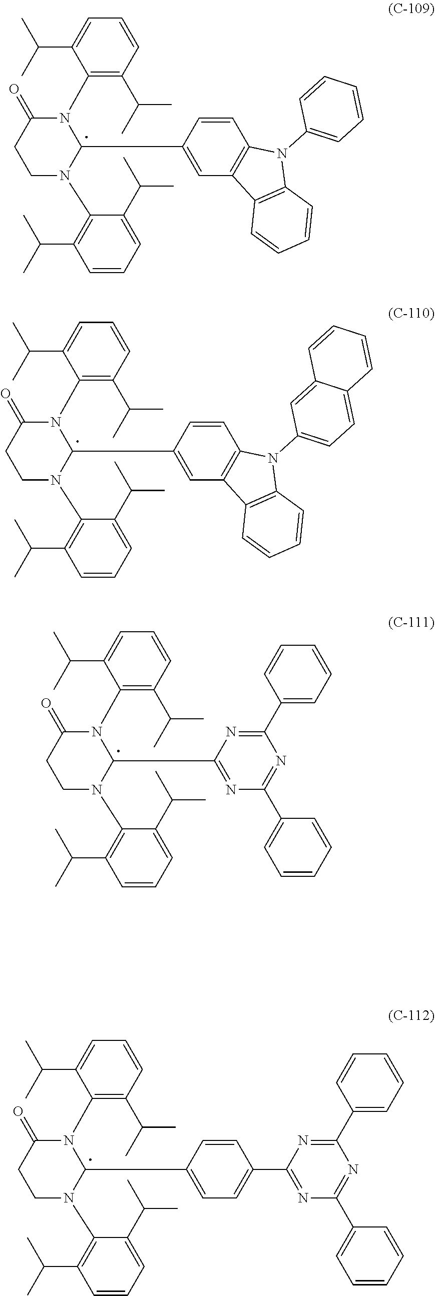

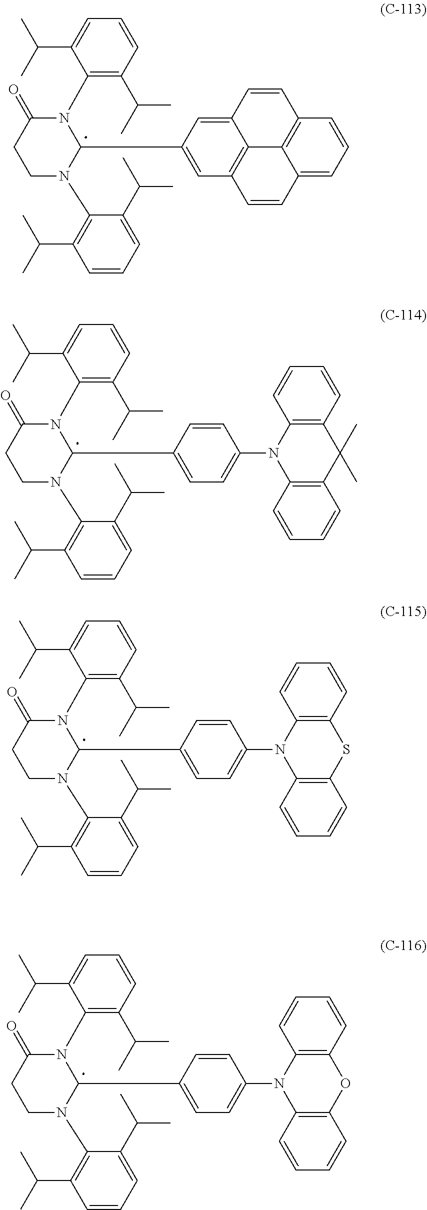

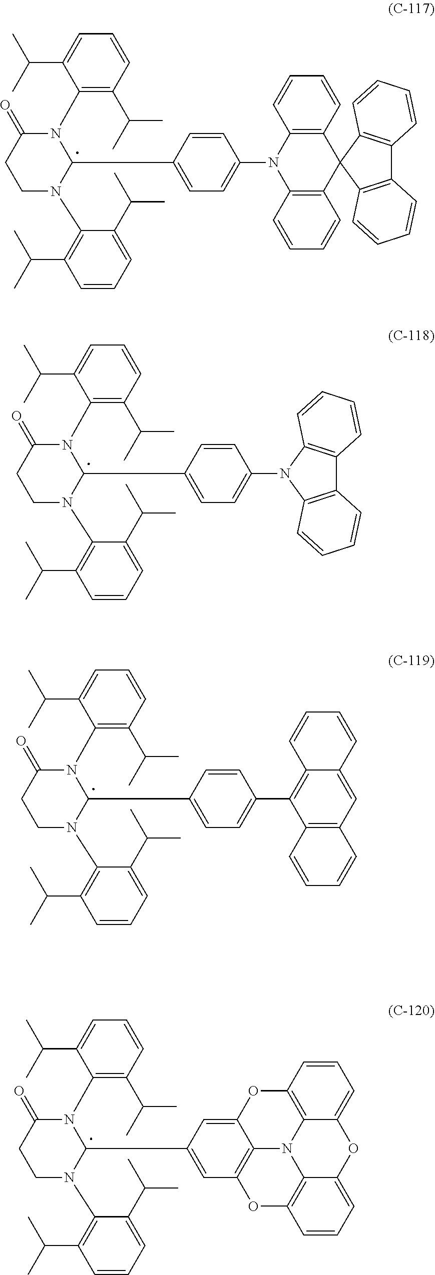

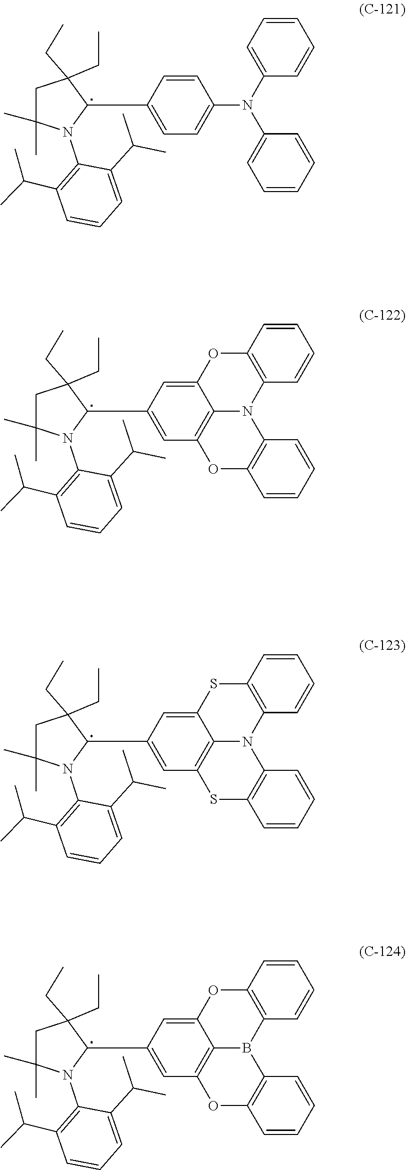

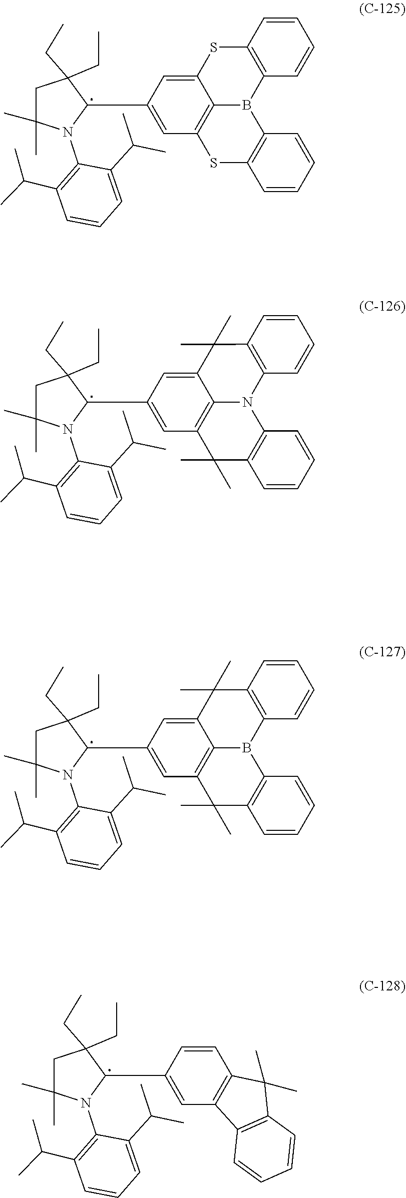

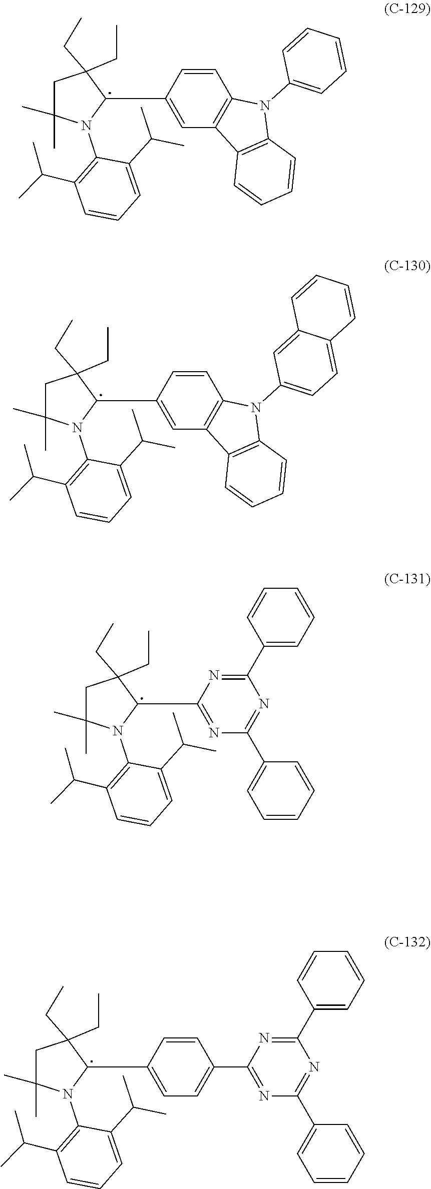

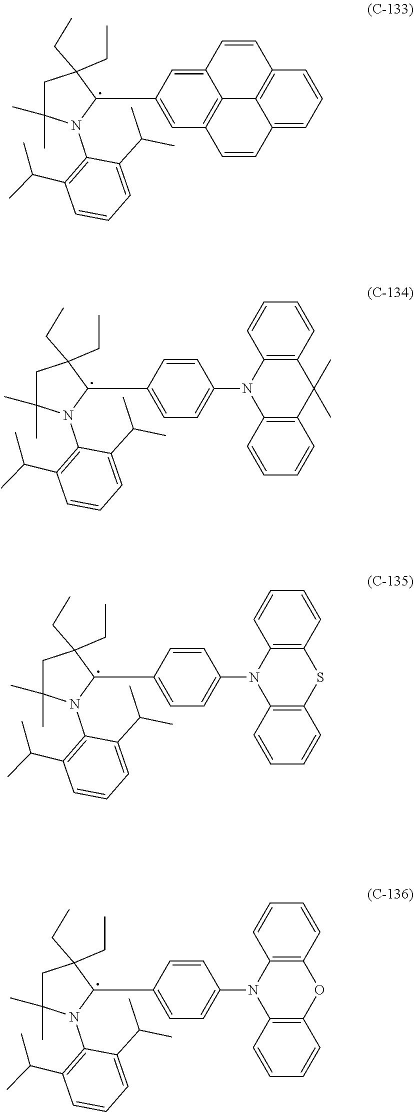

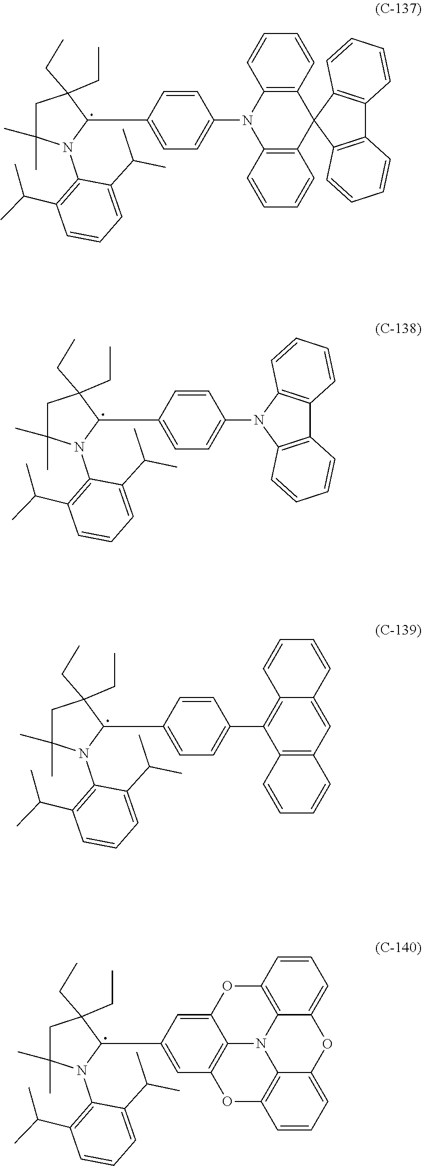

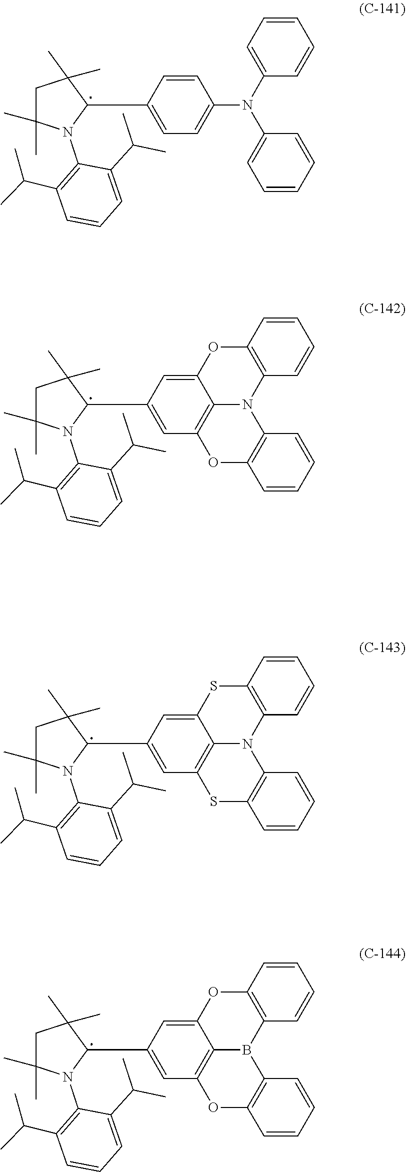

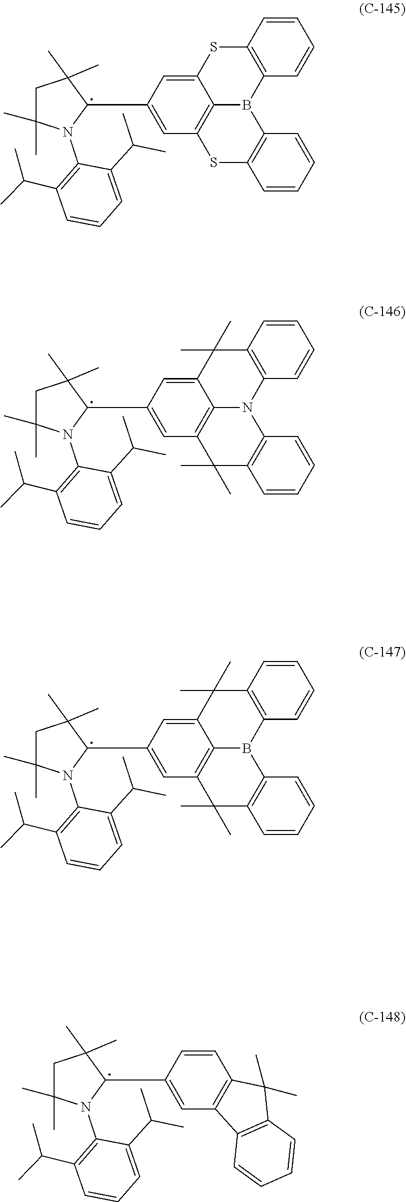

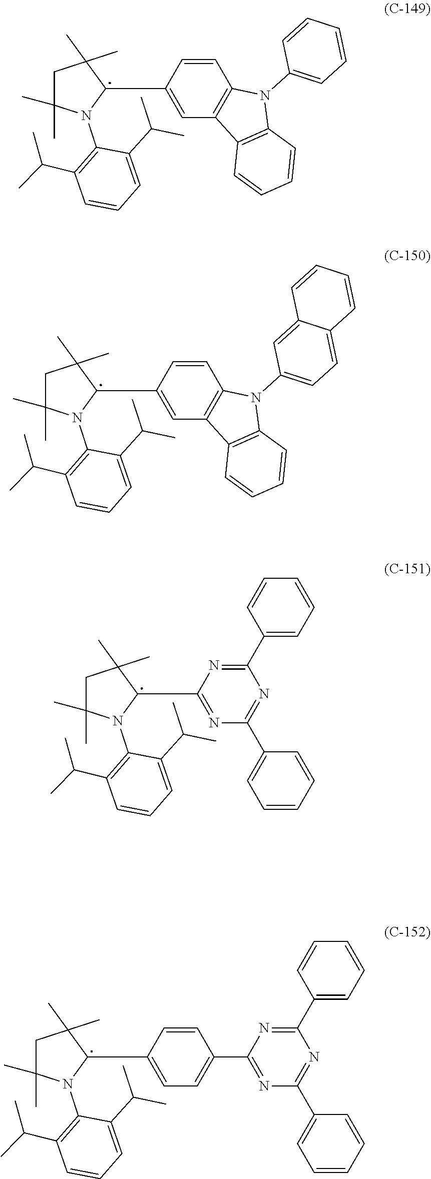

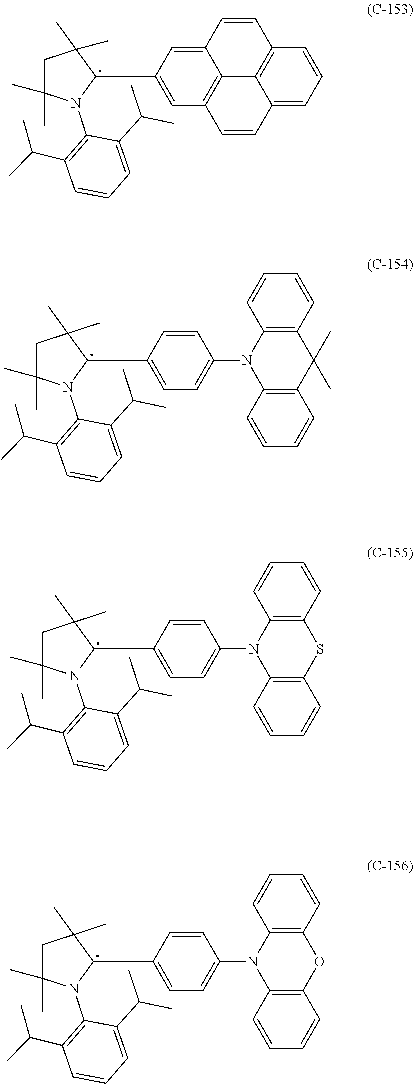

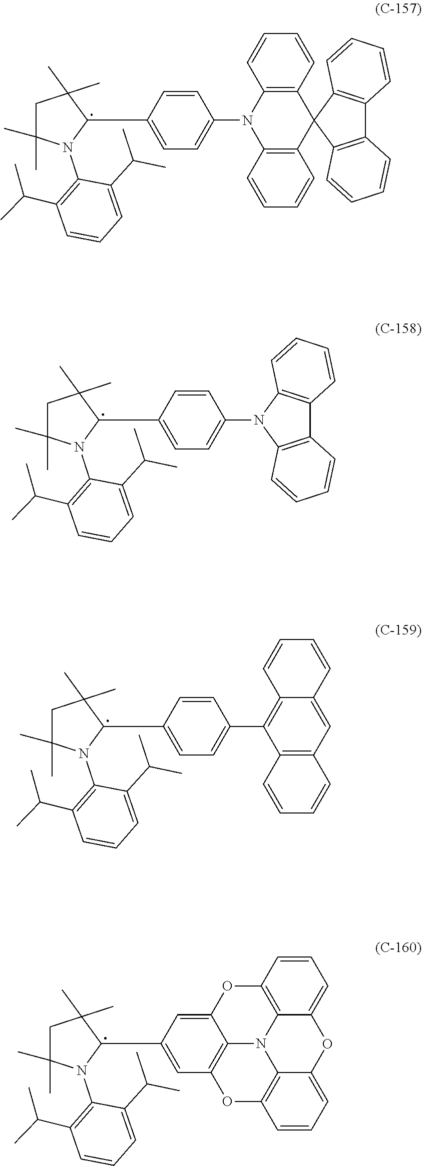

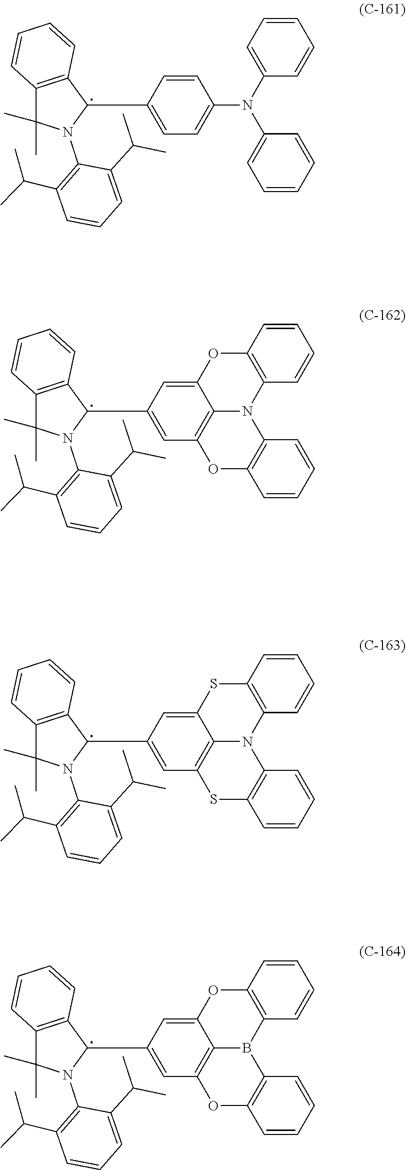

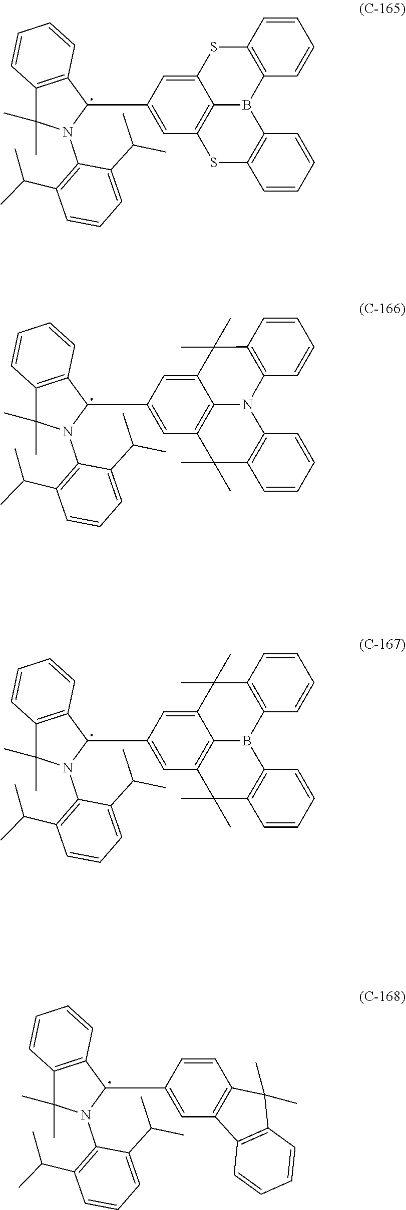

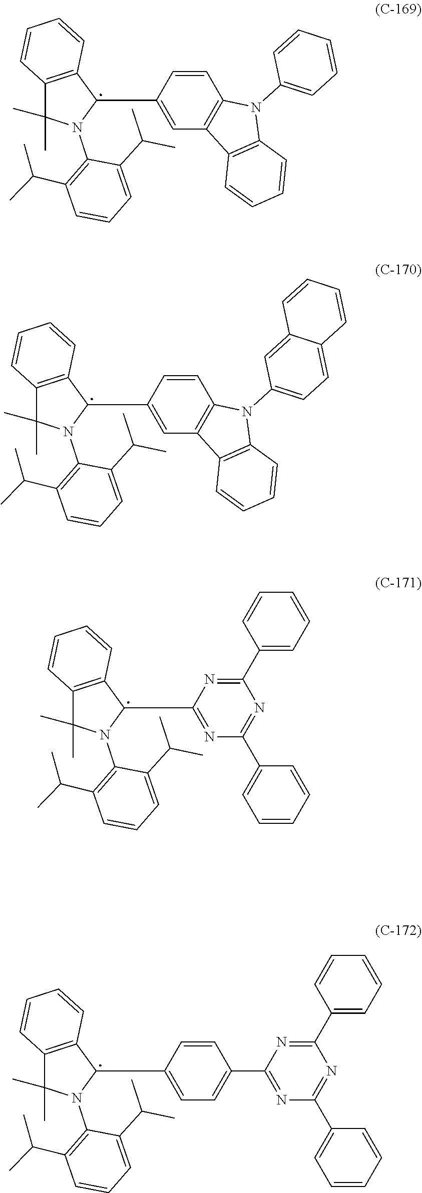

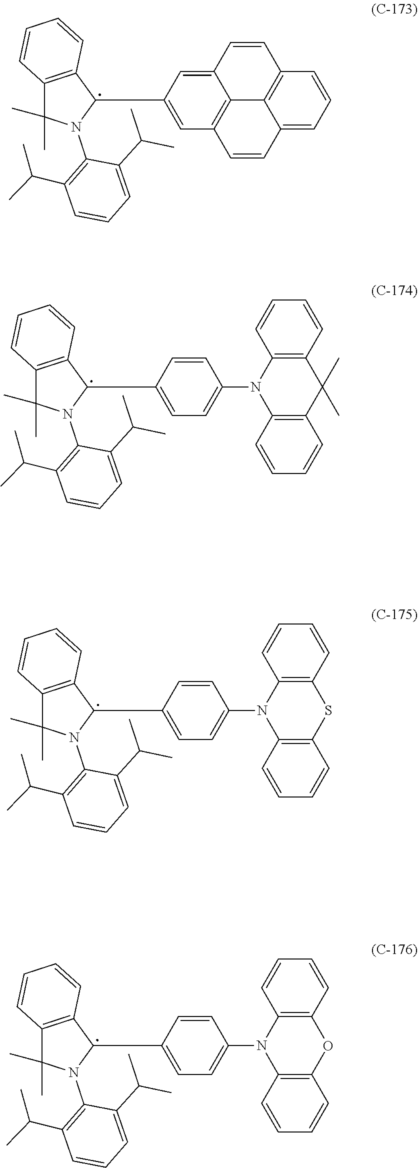

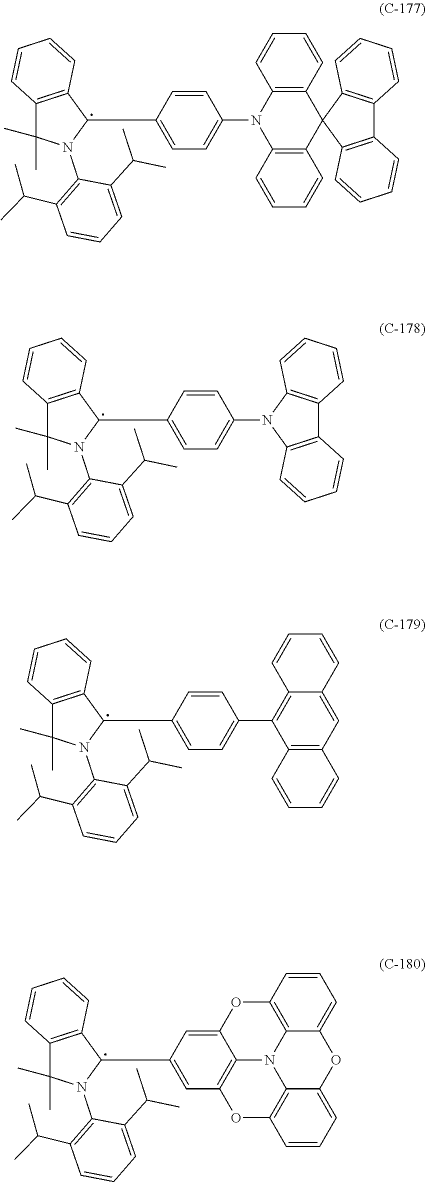

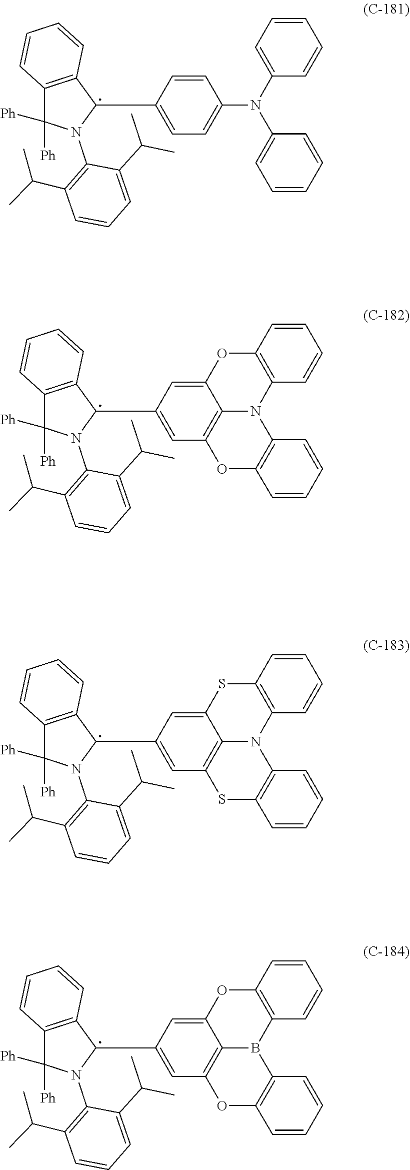

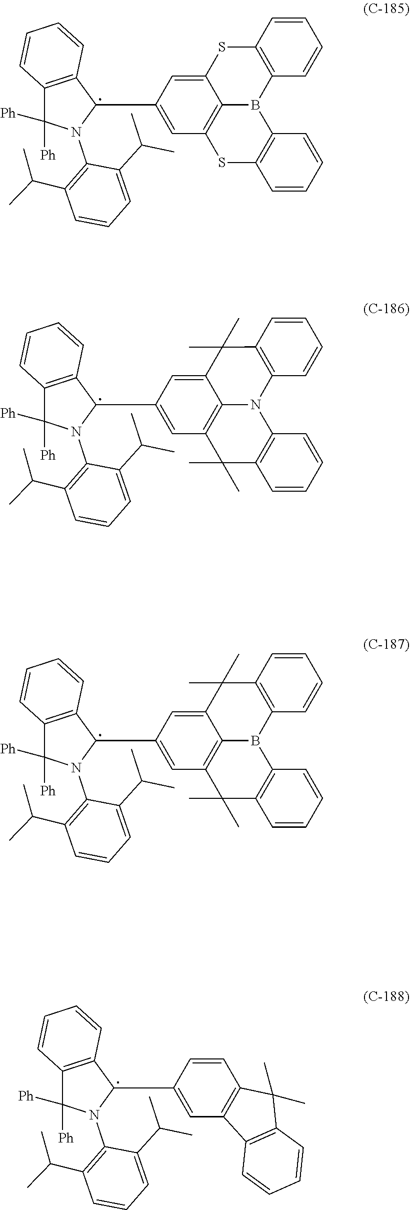

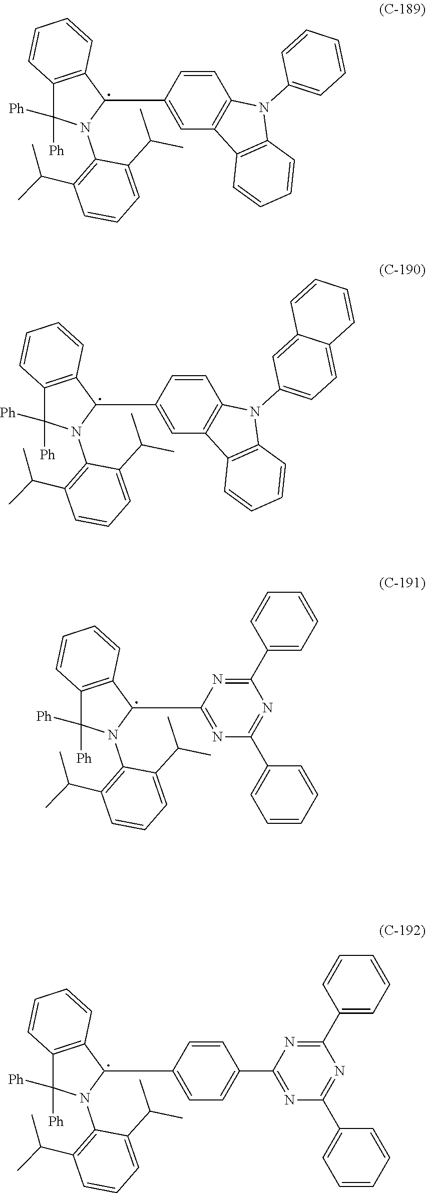

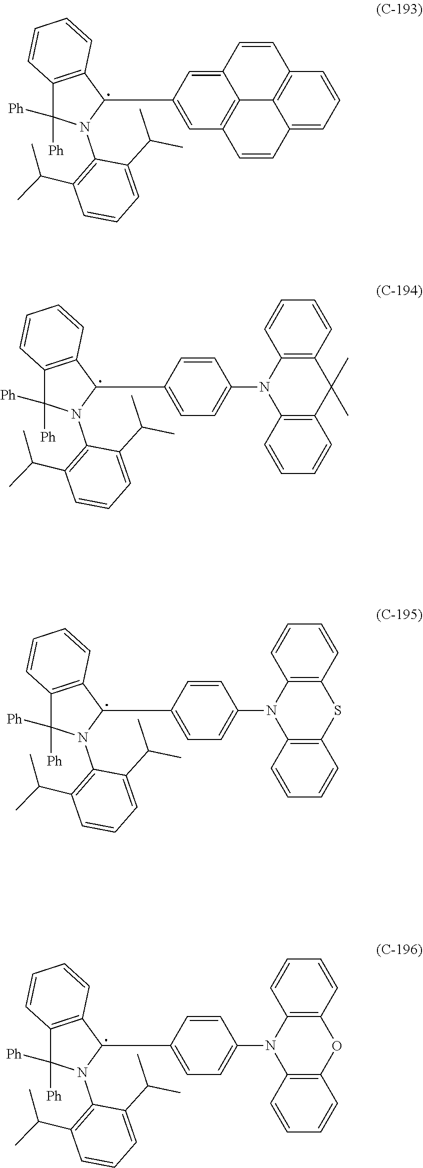

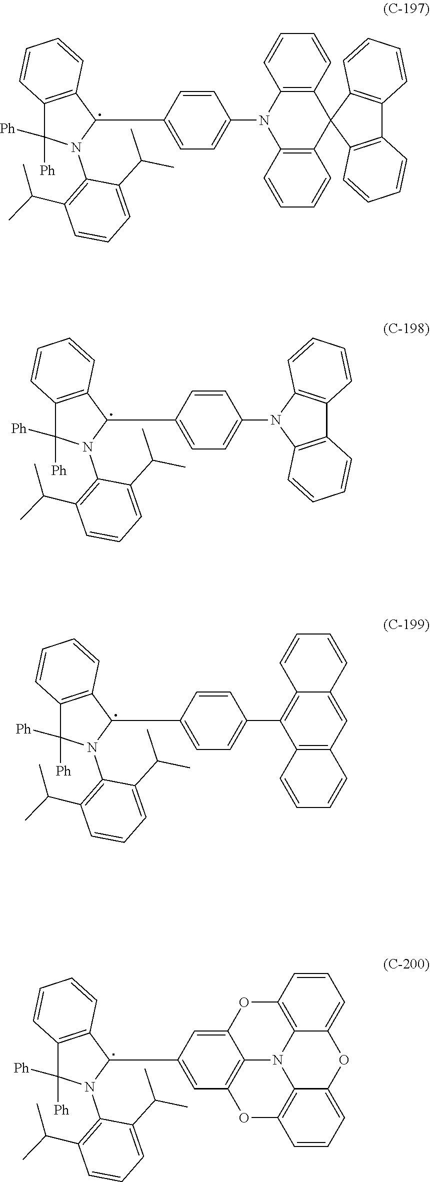

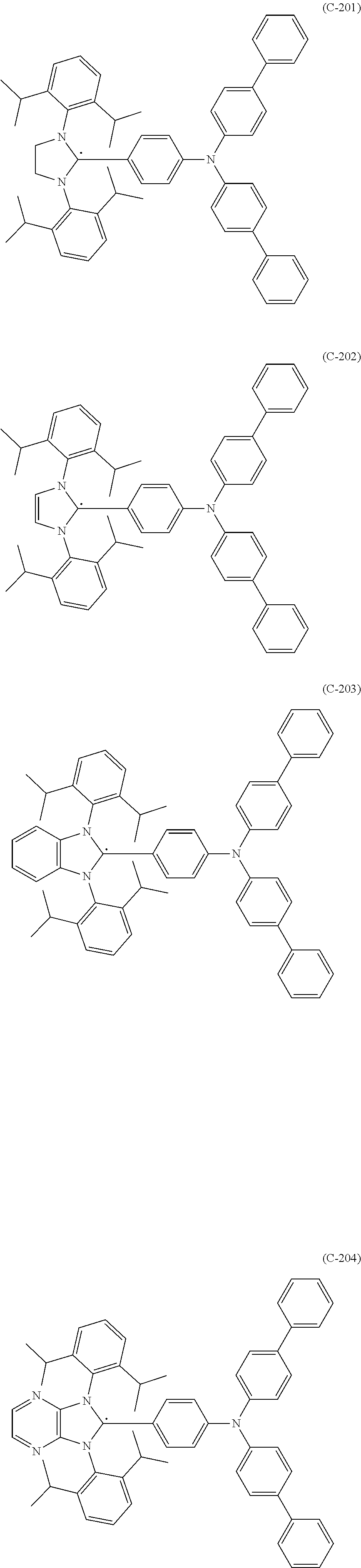

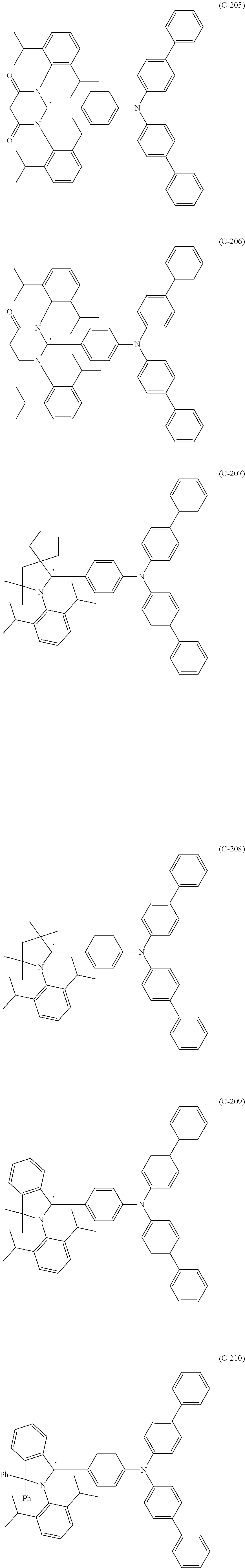

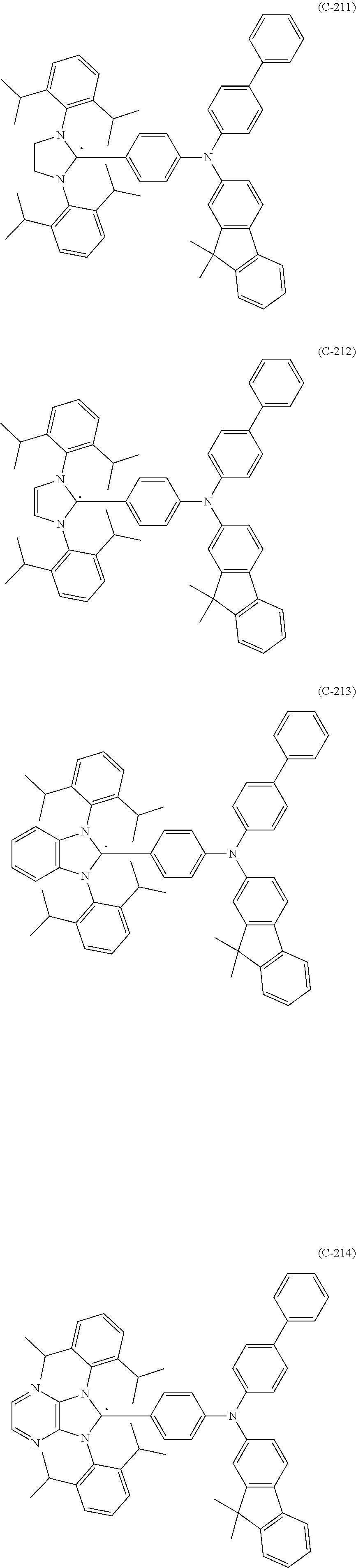

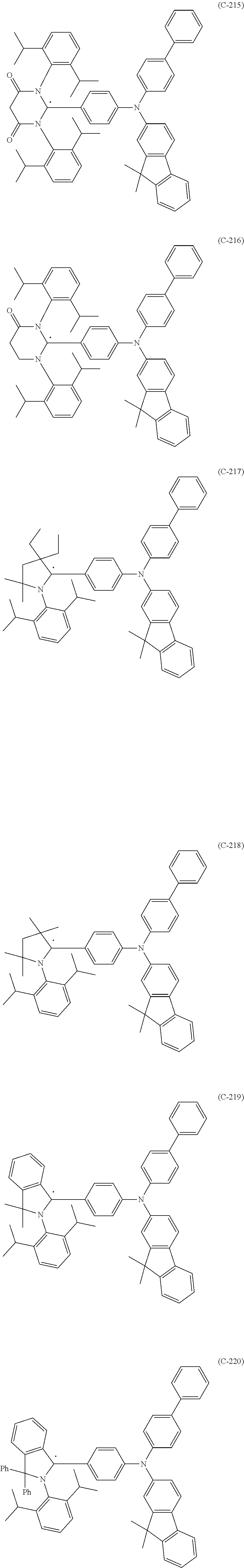

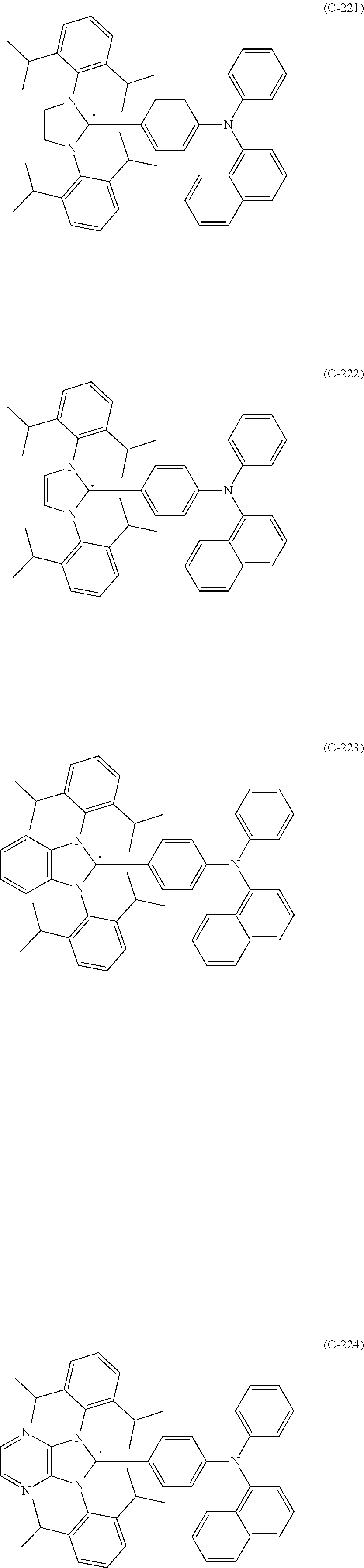

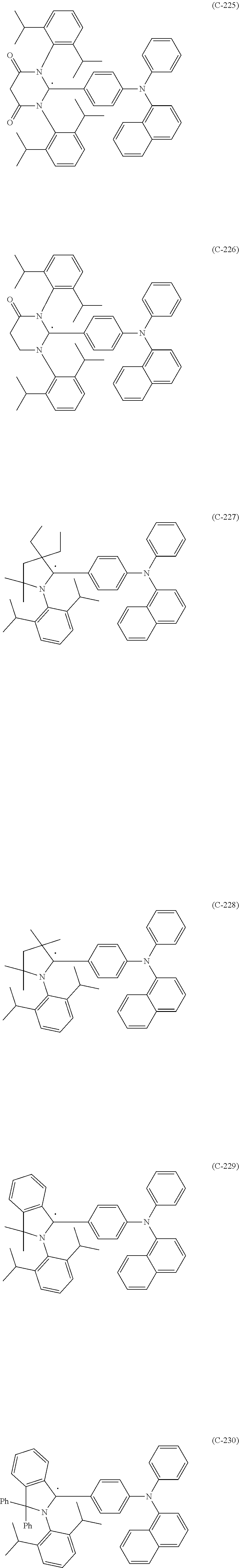

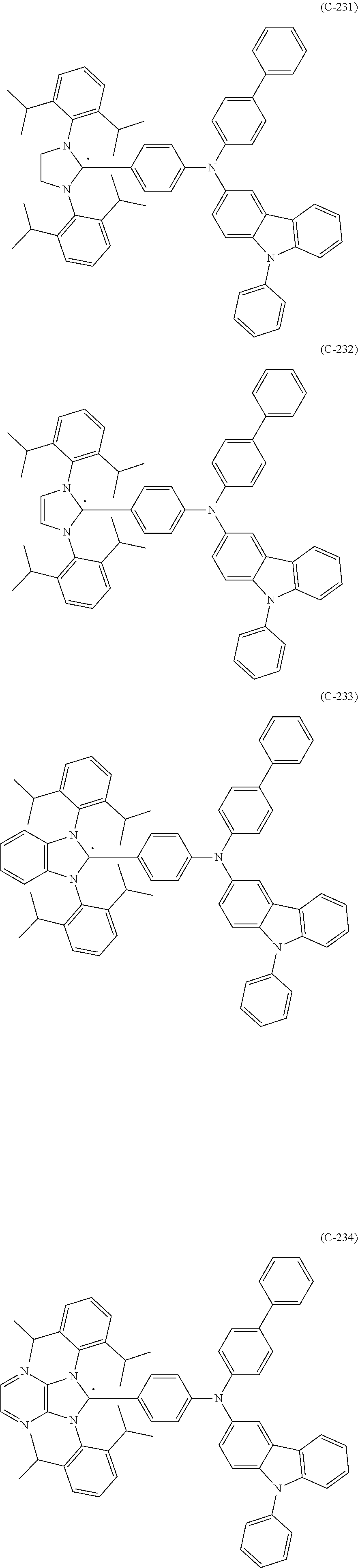

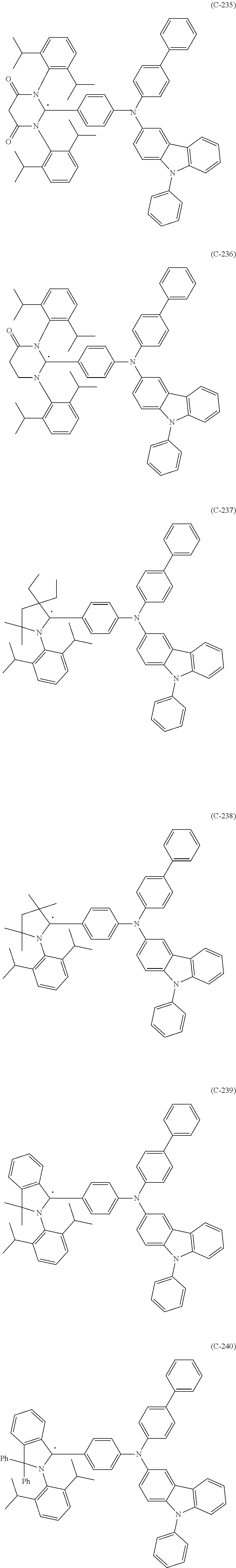

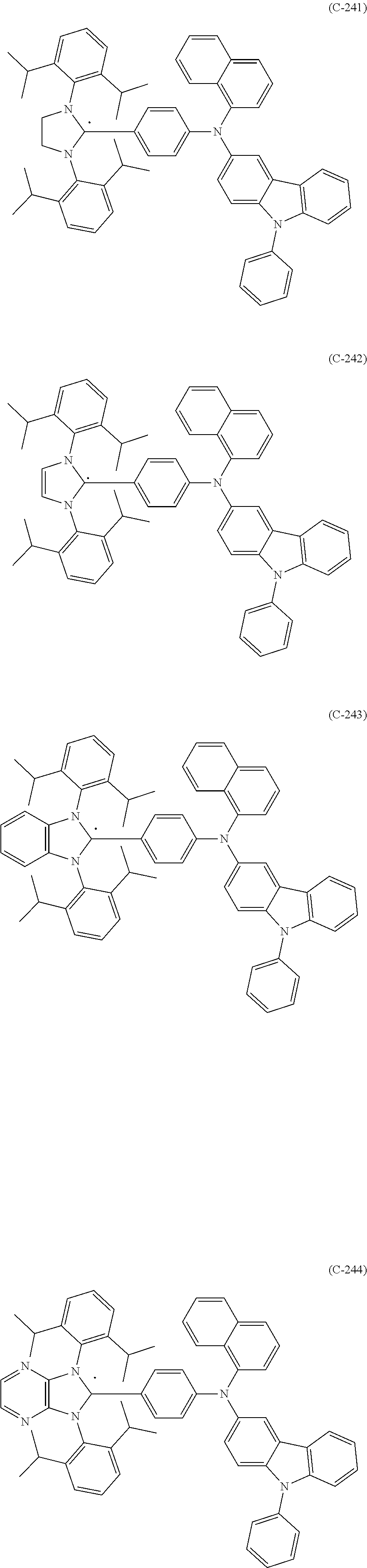

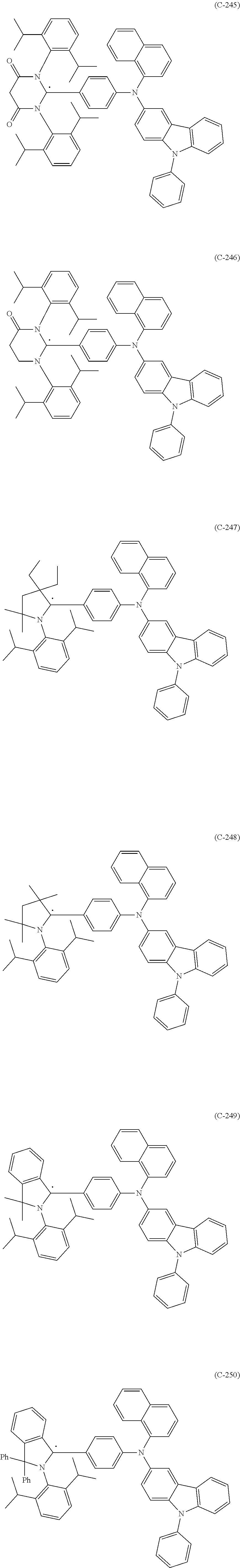

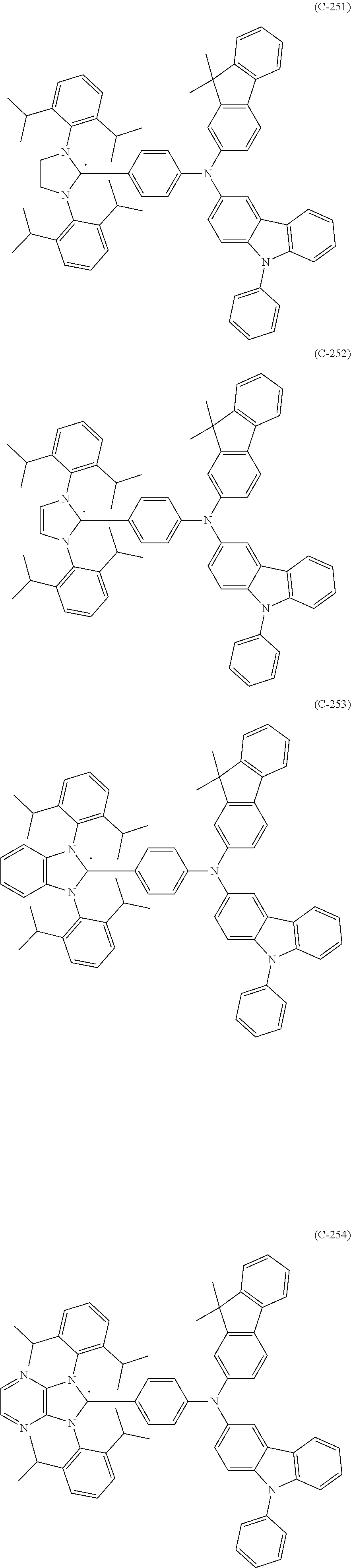

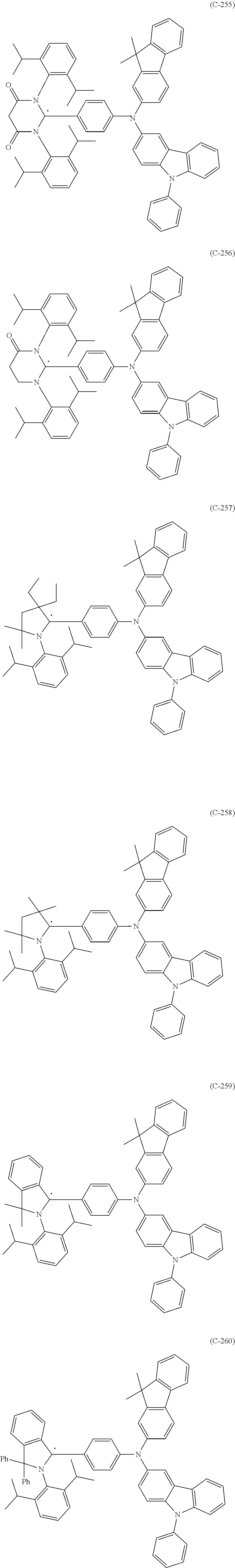

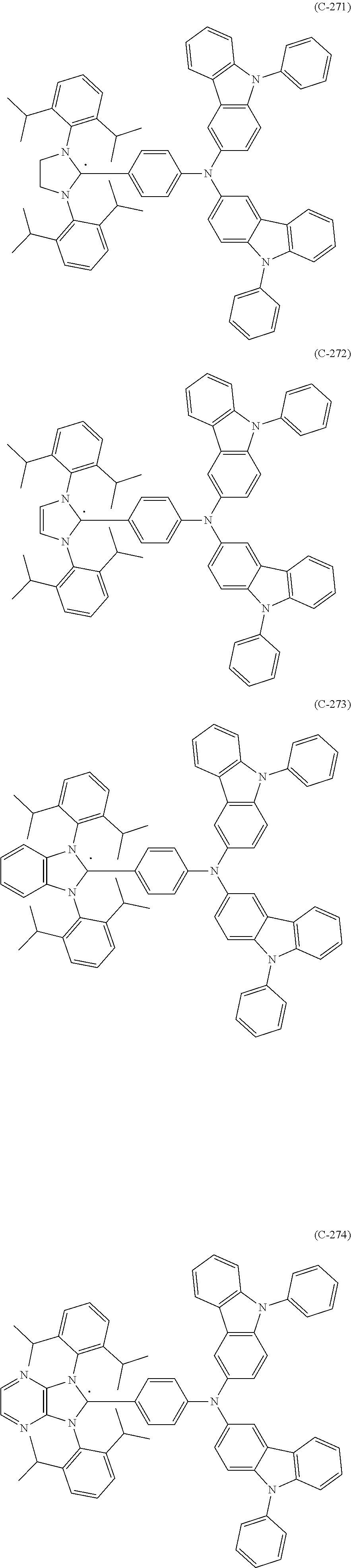

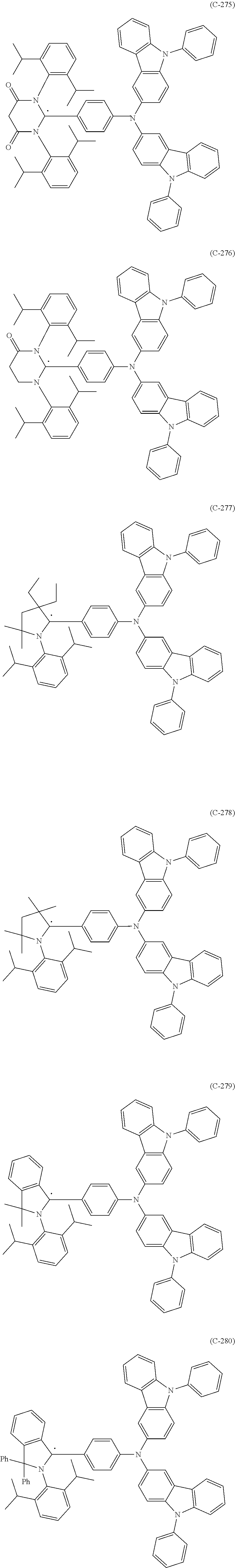

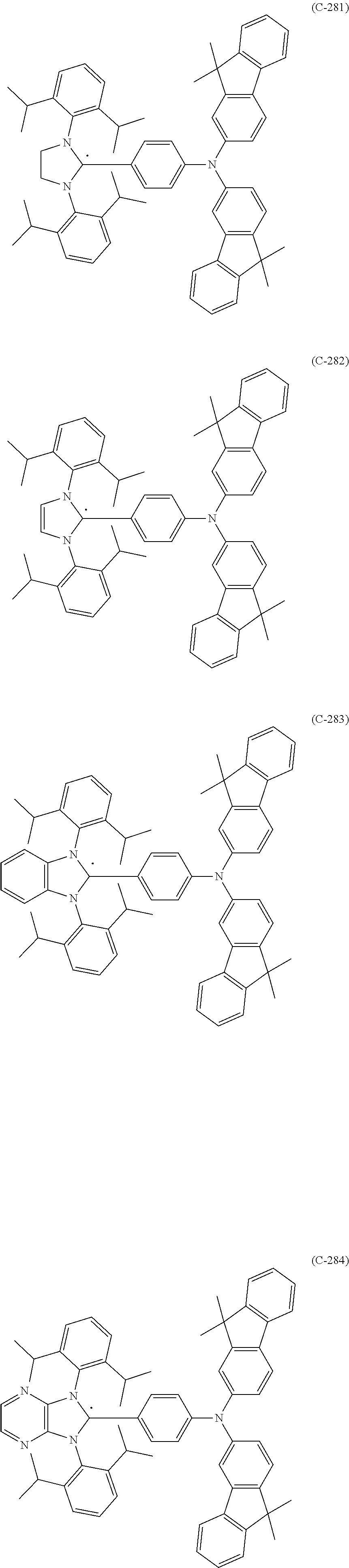

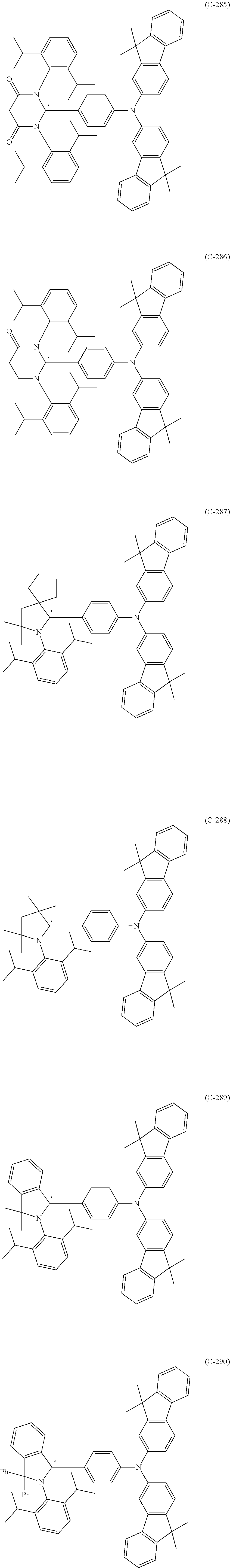

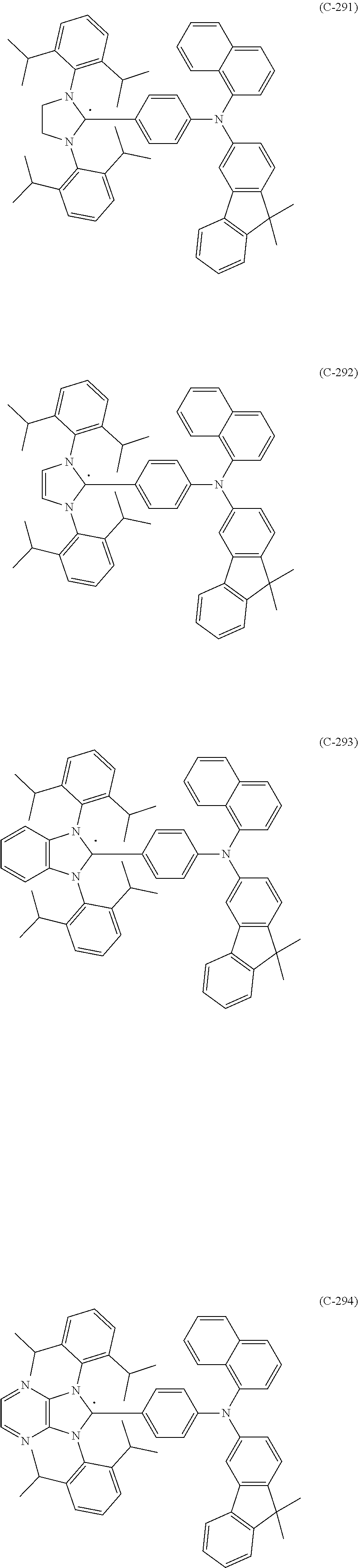

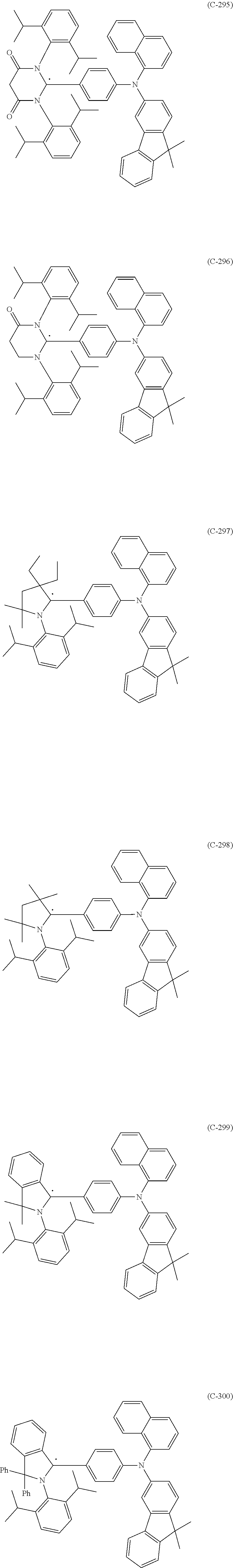

In some embodiments, the azacarbene carbon free radical molecule is an emitting material with an emitting wavelength between 300 nm and 5000 nm, preferably between 350 nm and 4000 nm, more preferably between 400 nm and 2000 nm. Herein the emission refers to photoluminescence or electroluminescence. In some embodiments, the photoluminescence quantum efficiency of the azacarbene carbon free radical molecule ≥30%, preferably >40%, more preferably ≥50%, and most preferably >60%.

In some embodiments, the azacarbene carbon free radical molecule may also be a non-emitting material.

In another aspect, the present disclosure also provides a polymer comprising a repeating unit, where the repeating unit comprises a structure of formula (I). In some embodiments, the polymer is a non-conjugated polymer in which the structural unit of formula (I) is on a side chain. In some embodiments, the polymer is a conjugated polymer.

In some embodiments, the polymer is a non-conjugated polymer in which the structural unit of formula (I) is on a side chain. In some embodiments, the polymer is a conjugated polymer.

In some embodiments, the synthetic method the polymer is selected from the group consisting of: SUZUKI-, YAMAMOTO-, STILLE-, NIGESHI-, KUMADA-, HECK-, SONOGASHIRA-, HIYAMA-, FUKUYAMA-, HARTWIG-BUCHWALD-, and ULLMAN-.

In some embodiments, the glass transition temperature (Tg) of the polymer ≥100° C., preferably ≥120° C., more preferably ≥140° C., further preferably ≥160° C., and most preferably ≥180° C.

In some embodiments, the polydispersity index (PDI) of the polymer is preferably from 1 to 5, more preferably 1 to 4, even more preferably 1 to 3, further preferably 1 to 2, and most preferably 1 to 1.5.

In some embodiments, the weight-average molecular weight (Mw) of the polymer is preferably from 10 k to 1 million, more preferably 50 k to 500 k, even more preferably 100 k to 400 k, further preferably 150 k to 300 k, and most preferably 200 k to 250 k.

In yet another aspect, the present disclosure further provides a mixture comprising at least one azacarbene carbon free radical molecule or polymer as described herein, and at least one other organic functional material.

The at least one other organic functional material may be selected from the group consisting of a hole-injection material (HIM), a hole-transport material (HTM), a hole-blocking material (HBM), an electron-injection material (EIM), an electron-transport material (ETM), an electron-blocking material (EBM), a host material (Host), an emitting material, a singlet emitting material (fluorescent emitting material), a thermally activated delayed fluorescence material (TADF material), a triplet emitting material (phosphorescent emitting material), and in particular from a luminescent metal-organic complex or an organic dye. These organic functional materials are described in detail, for example, in WO2010135519A1, US20090134784A1 and WO2011110277A1. The entire contents of these three documents are incorporated herein by reference in their entirety.

It is an object of the present disclosure to provide a material for the evaporation-based organic electronic devices.

In some embodiments, the molecular weight of the azacarbene carbon free radical molecule ≤1100 g/mol, preferably ≤1000 g/mol, more preferably ≤950 g/mol, further preferably ≤900 g/mol, and most preferably ≤800 g/mol.

Another object of the present disclosure is to provide a material for the printed organic electronic devices.

In some embodiments, the molecular weight of the azacarbene carbon free radical molecule ≥700 g/mol, preferably >900 g/mol, more preferably ≥1000 g/mol, and most preferably ≥1100 g/mol.

In some embodiments, the azacarbene carbon free radical molecule as described herein has a solubility of ≥10 mg/mL in toluene at 25° C., preferably ≥15 mg/mL, and most preferably ≥20 mg/mL.

In yet another aspect, the present disclosure further provides a formulation or a ink comprising at least one azacarbene carbon free radical molecule or polymer as described herein, and at least one organic solvent.

The viscosity and surface tension of the ink are important parameters in printing processes. A suitable ink surface tension is required for the specific substrates and the specific printing methods.

In some embodiments, the surface tension of the ink as described herein at 25° C. is in the range of 19 dyne/cm to 50 dyne/cm; more preferably in the range of 22 dyne/cm to 35 dyne/cm; and most preferably in the range of 25 dyne/cm to 33 dyne/cm.

In some embodiments, the viscosity of the ink as described herein at 25° C. is in the range of from about 1 cps to 100 cps; particularly in the range of 1 cps to 50 cps; more particularly in the range of 1.5 cps to 20 cps; and most particularly in the range of 4 cps to 20 cps. The resulting formulation will be particularly suitable for ink-jet printing.

The viscosity can be adjusted by different methods, such as by the selection of appropriate solvent and the concentration of the functional materials in the ink. In the ink comprising the above-mentioned azacarbene carbon free radical molecules or polymers as described herein facilitate the adjustment of the printing ink in the appropriate range according to the printing method used. Generally, in the formulation comprising the functional material as described herein, the weight ratio of the functional material ranges from 0.3 wt % to 30 wt %, preferably from 0.5 wt % to 20 wt %, more preferably from 0.5 wt % to 15 wt %, further preferably from 0.5 wt % to 10 wt %, and most preferably from 1 wt % to 5 wt %.

In some embodiments, the at least one organic solvent of the ink as described herein is selected from aromatic-based or heteroaromatic-based solvents, particular in aliphatic chain/ring substituted aromatic solvents, aromatic ketone solvents, or aromatic ether solvents.

Examples of solvents suitable for the present disclosure include, but not limited to aromatic-based or heteroaromatic-based solvents, such as p-diisopropylbenzene, amylbenzene, tetralin, cyclohexylbenzene, chloronaphtalene, 1,4-dimethylnaphthalene, 3-isopropylbenzene, p-methylisopropylbenzene, dipentylbenzene, tripentylbenzene, pentyltoluene, o-xylene, m-xylene, p-xylene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, p-diiisopropylbenzene, 1-methoxynaphthalene, cyclohexylbenzene, dimethylnaphthalene, 3-isopropylbiphenyl, p-methylisopropylbenzene, 1-methylnaphthalene, 1,2,4-trichlorobenzene, 1,3-dipropoxybenzene, 4,4-difluorobenzenemethane, 1,2-dimethoxy-4-(1-propenyl)benzene, diphenylmethane, 2-phenylpyridine, 3-phenylpyridine, N-methyldiphenylamine, 4-isopropylbipheny, α,α-dichlorodiphenylmethane, 4-(3-phenylpropyl)pyridine, benzyl benzoatel, 1,1-bis(3,4-dimethylphenyl) ethane, 2-isopropylnaphthalene, dibenzyl ether, etc.; ketone-based solvents, such as 1-tetrahydronaphthalone, 2-tetrahydronaphthalone, 2-(phenylepoxy)tetrahydronaphthalone, 6-(methoxy)tetrahydronaphthalone, acetophenone, phenylacetone, benzophenone, and derivatives thereof such as 4-methyl acetophenone, 3-methyl acetophenone, 2-methyl acetophenone, 4-methyl propanone, 3-methyl propanone, 2-methyl propanone, isophorone, 2,6,8-trimethyl-4-nonanone, fenchone, 2-nonanone, 3-nonanone, 5-nonanone, 2-decanone, 2,5-hexanedione, phoron, di-n-amyl ketone, etc; aromatic ether solvents, such as 3-phenoxytoluene, butoxybenzene, benzylbutylbenzene, p-anisaldehyde dimethyl acetal, tetrahydro-2-phenoxy-2H-pyran, 1,2-dimethoxy-4-(1-propenyl)benzene, 1,4-benzodioxane, 1,3-dipropylbenzene, 2,5-dimethoxytoluene, 4-ethylphenyl ether, 1,2,4-trimethoxybenzene, 4-(1-propenyl)-1,2-dimethoxybenzene, 1,3-dimethoxybenzene, glycidyl phenyl ether, dibenzyl ether, 4-tert-butyl anisole, trans-anethole, 1,2-dimethoxybenzene, 1-methoxynaphthalene, diphenyl ether, 2-phenoxymethyl ether, 2-phenoxytetrahydrofuran, ethyl-2-naphthyl ether, dipentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether, etc.; ester solvent, such as alkyl octanoate, alkyl sebacate, alkyl stearate, alkyl benzoate, alkyl phenylacetate, alkyl cinnamate, alkyl oxalate, alkyl maleate, alkyl lactone, alkyl oleate, etc.

Further, the at least one organic solvent of the ink as described herein can be selected from aliphatic ketones, such as 2-nonanone, 3-nonanone, 5-nonanone, 2-decanone, 2,5-hexanedione, 2,6,8-trimethyl-4-nonanone, phorone, 6-undecanone, etc.; and the at least one organic solvent of the ink can be selected from aliphatic ethers, such as, dipentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether, etc.

In some embodiments, the printing ink further comprises another organic solvent. Examples of the another organic solvents include, but not limited to: methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 3-phenoxytoluene, 1,1,1-trichloroethane, 1,1,2,2-tetrachloroethane, ethyl acetate, butyl acetate, dimethylformamide, dimethylacetamide, dimethylsulfoxide, tetraline, decalin, indene, and/or mixtures thereof.

In some embodiments, the formulation as described herein is a solution.

In some embodiments, the formulation as described herein is a dispersion.

The formulations of the embodiments may comprise the azacarbene carbon free radical molecule or the polymer or the mixture of 0.01 wt % to 20 wt %, preferably 0.1 wt % to 15 wt %, more preferably 0.2 wt % to 10 wt %, and most preferably 0.25 wt % to 5 wt %.

The present disclosure further provides the use of the formulation as a coating or printing ink in the preparation of organic electronic devices, particularly preferably by printing or coating processing methods.

Where suitable printing or coating techniques include, but not limited to ink-jet printing, nozzle printing, gravure printing, typographic printing, screen printing, dip coating, spin coating, blade coating, roller printing, torsion roll printing, planographic printing, flexographic printing, rotary printing, spray coating, brush coating, pad printing, slit die coating, and so on. Preferred techniques are ink-jet printing, nozzle printing, and gravure printing. The solution or dispersion may additionally comprise one or more components, such as surface active compounds, lubricants, wetting agents, dispersing agents, hydrophobic agents, binders, etc., which are used to adjust the viscosity and film forming properties, or to improve adhesion, etc. For more information on printing technologies and their requirements for solutions, such as solvent, concentration, viscosity, etc., please refer to Handbook of Print Media: Technologies and Production Methods, edited by Helmut Kipphan, ISBN 3-540-67326-1.

Based on the azacarbene carbon free radical molecule or the polymer, the present disclosure also provides an application of the azacarbene carbon free radical molecule or the polymer as described herein, i.e., the azacarbene carbon free radical molecule or the polymer is applied to an organic electronic device, and the organic electronic device may be selected from, but not limited to, an organic light-emitting diode (OLED), an organic photovoltaic cell (OPV), an organic light emitting electrochemical cell (OLEEC), an organic field effect transistor (OFET), an organic light emitting field effect transistor, an organic laser, an organic spintronic electronic device, an organic spin-valve, a photodiode, an organic sensor, an organic plasmon emitting diode (OPED), etc., particularly preferably is an organic electroluminescent device, such as an OLED, an OLEEC, an organic light emitting field effect transistor. In the embodiments of the present disclosure, it is preferred to use the azacarbene carbon free radical molecule or the polymer for the light-emitting layer of the organic electroluminescent device.

In yet another aspect, the present disclosure further provides an organic electronic device comprising at least one azacarbene carbon free radical molecule or polymer or mixture as described herein. Generally, such organic electronic device comprises a cathode, an anode, and a functional layer disposed between the cathode and the anode, where the functional layer comprises at least one azacarbene carbon free radical molecule or polymer or mixture as described herein. The organic electronic device may be selected from, but not limited to, an organic light-emitting diode (OLED), an organic photovoltaic cell (OPV), an organic light emitting electrochemical cell (OLEEC), an organic field effect transistor (OFET), an organic light emitting field effect transistor, an organic laser, an organic spintronic device, an organic spin-valve, a photodiode, an organic sensor, an organic plasmon emitting diode (OPED), etc., particularly preferably is an organic electroluminescent device, such as an OLED, an OLEEC, an organic light emitting field effect transistor; others are particularly selected from organic spintronic devices or organic spin-valves.

In some embodiments, the organic electroluminescent device comprises a light-emitting layer, where the light-emitting layer comprises an azacarbene carbon free radical molecule or a polymer or a mixture as described herein; or comprises an azacarbene carbon free radical molecule or a polymer or a mixture as described herein, and a phosphorescent emitter; or comprises an azacarbene carbon free radical molecule or a polymer or a mixture as described herein, and a host material; or comprises an azacarbene carbon free radical molecule or a polymer or a mixture as described herein, a phosphorescent emitter, and a host material.

In the organic electroluminescent devices as described herein, in particular an OLED, which comprises a substrate, an anode, at least one light-emitting layer, and a cathode.

The substrate should be opaque or transparent. A transparent substrate could be used to produce a transparent light-emitting device (for example: Bulovic et al., Nature, 1996, 380, p29, and Gu et al., Appl. Phys. Lett., 1996, 68, p 2606). The substrate can be rigid or flexible, e.g. it can be plastic, metal, semiconductor wafer, or glass. Preferably, the substrate has a smooth surface. Particularly ideal are substrates without surface defects. In some embodiments, the substrate is flexible and can be selected from a polymer film or plastic with a glass transition temperature (Tg)>150° C., preferably >200° C., more preferably >250° C., and most preferably >300° C. Examples of the suitable flexible substrates include poly ethylene terephthalate (PET) and polyethylene glycol (2,6-naphthalene) (PEN).

The anode may be a conductive metal, or a metal oxide, or a conductive polymer. The anode should be able to easily inject holes into a hole-injection layer (HIL), a hole-transport layer (HTL), or a light-emitting layer. In some embodiments, the absolute value of the difference between the work function of the anode and the HOMO energy level/valence band energy level of the emitter of the light-emitting layer or the p-type semiconductor materials of the hole-injection layer (HIL)/hole-transport layer (HTL)/electron-blocking layer (EBL) <0.5 eV, preferably <0.3 eV, and most preferably <0.2 eV. Examples of anode materials may include, but not limited to: Al, Cu, Au, Ag, Mg, Fe, Co, Ni, Mn, Pd, Pt, ITO, aluminum-doped zinc oxide (AZO), etc. Other suitable anode materials are known and can be readily selected for use by the general technicians in this field. The anode materials can be deposited using any suitable technique, such as a suitable physical vapor deposition method including RF magnetron sputtering, vacuum thermal evaporation, e-beam, etc. In some embodiments, the anode is patterned. Patterned conductive ITO substrates are commercially available and can be used to produce the devices as described herein.

The cathode may be a conductive metal or a metal oxide. The cathode should be able to easily inject electrons into the electron-injection layer (EIL), the electron-transport layer (ETL), or the directly into the light-emitting layer. In some embodiments, the absolute value of the difference between the work function of the cathode and the LUMO energy level/conduction band energy level of the emitter of the light-emitting layer, or the n-type semiconductor materials of the electron-injection layer (EIL)/electron-transport layer (ETL)/hole-blocking layer (HBL) <0.5 eV, preferably <0.3 eV, and most preferably <0.2 eV. In principle, all materials those can be used as cathodes for OLEDs may be applied as cathode materials for the devices as described herein. Examples of cathode materials include, but not limited to: Al, Au, Ag, Ca, Ba, Mg, LiF/Al, MgAg alloy, BaF₂/Al, Cu, Fe, Co, Ni, Mn, Pd, Pt, ITO, etc. The cathode materials can be deposited using any suitable technique, such as the suitable physical vapor deposition method including RF magnetron sputtering, vacuum thermal evaporation, e-beam, etc.

The OLED may also comprise other functional layers, such as a hole-injection layer (HIL), a hole-transport layer (HTL), an electron-blocking layer (EBL), an electron-injection layer (EIL), an electron-transport layer (ETL), and a hole-blocking layer (HBL). Materials suitable for use in these functional layers are described in details above and in WO2010135519A1, US20090134784A1 and WO2011110277A1. The entire contents of these three documents are hereby incorporated herein for reference.

In some embodiments, the light-emitting layer of the light-emitting device is prepared by the formulation as described herein.

The emitting wavelength of the light-emitting device as described herein is between 300 nm and 5000 nm, preferably between 350 nm and 4000 nm, more preferably between 400 nm and 2000 nm.

The present disclosure further provides the applications of organic electronic devices in various electronic equipment, including, but not limited to, display devices, lighting equipment, light sources, sensors, etc.

The present disclosure further provides electronic devices comprising organic electronic devices of the present disclosure, including, but not limited to, display devices, lighting equipment, light sources, sensors, etc.

The present disclosure will be described below in conjunction with the preferred embodiments, but the present disclosure is not limited to the following embodiments. It should be understood that the scope of the present disclosure is covered by the scope of the claims of the present disclosure, and those skilled in the art should understand that certain changes may be made to the embodiments of the present disclosure.

### Specific Embodiment

### 1. Synthesis

### Synthesis Example 1: Synthesis of Azacarbene Carbon Free Radical C-001

The synthetic route of the azacarbene carbon free radical C-001 is as follows:

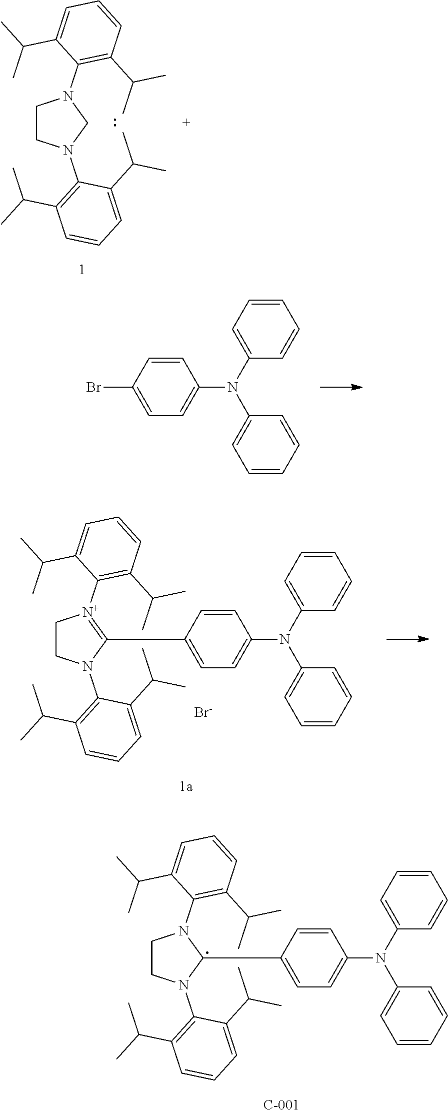

### Synthesis of Compound 1a:

Carbene 1 (0.031 g, 0.08 mmol), 4-bromotriphenylamine (0.029 g, 0.09 mmol), and Ni(cod)₂ (1.1 mg, 0.004 mmol, 5 mol %) were added to a dry double-necked flask. After adding 8 mL of dry o-xylene under N₂ atmosphere, the resulting mixture was stirred at room temperature for 10 min, heated to reflux, then stirred for 4 h. After cooling down to room temperature, the organic phases were concentrated, then the resulting sample was purified by column chromatography (eluent: dichloromethane:ethanol=10:1) to yield 0.034 g (60% yield) of compound 1a (yellow solid).

### Synthesis of Azacarbene Carbon Free Radical C-001:

Compound 1a (0.014 mg, 0.02 mmol) and KC8 (0.003 g, 0.02 mmol) were added to a dry schlenck tube. After adding 15 mL of dry-cooled tetrahydrofuran under N₂ atmosphere, the resulting mixture was stirred at −78° C. for 1 h, then the result was heated to room temperature and stirred for 2 h. After the reaction was completed, the result was filtrated, then the filtrate was concentrated and dried to yield 0.011 g (90% yield) of azacarbene carbon free radical C-001 (solid).

### Synthesis Example 2: Synthesis of Azacarbene Carbon Free Radical C-002

The synthetic route of the azocarbene carbon free radical C-002 is as follows:

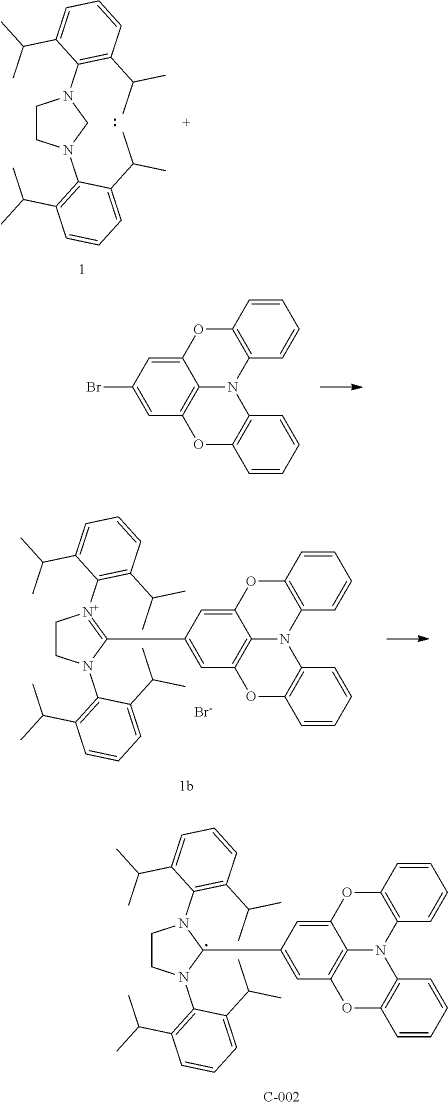

### Synthesis of Compound 1b:

Carbene 1 (0.031 g, 0.08 mmol), 4-bromo-dioxotriphenylamine (0.032 g, 0.09 mmol), and Ni(cod)₂ (1.1 mg, 0.004 mmol, 5 mol %) were added to a dry double-necked flask. After adding 8 mL of dry o-xylene under N₂ atmosphere, the resulting mixture was stirred at room temperature for 10 min, heated to reflux, then stirred for 4 h. After cooling down to room temperature, the organic phases were concentrated, then the resulting sample was purified by column chromatography (eluent: dichloromethane:ethanol=10:1) to yield 0.033 g (55% yield) of compound 1b (yellow solid).

### Synthesis of Azacarbene Carbon Free Radical C-002:

Compound 1b (0.015 mg, 0.02 mmol) and KC8 (0.003 g, 0.02 mmol) were added to a dry schlenck tube. After adding 15 mL of dry-cooled tetrahydrofuran under N₂ atmosphere, the resulting mixture was stirred at −78° C. for 1 h, then the result was heated to room temperature and stirred for 2 h. After the reaction was completed, the result was filtrated, then the filtrate was concentrated and dried to yield 0.012 g (90% yield) of azacarbene carbon free radical C-002 (solid).

### Synthesis Example 3: Synthesis of Azacarbene Carbon Free Radical C-003

The synthetic route of the azacarbene carbon free radical C-003 is as follows:

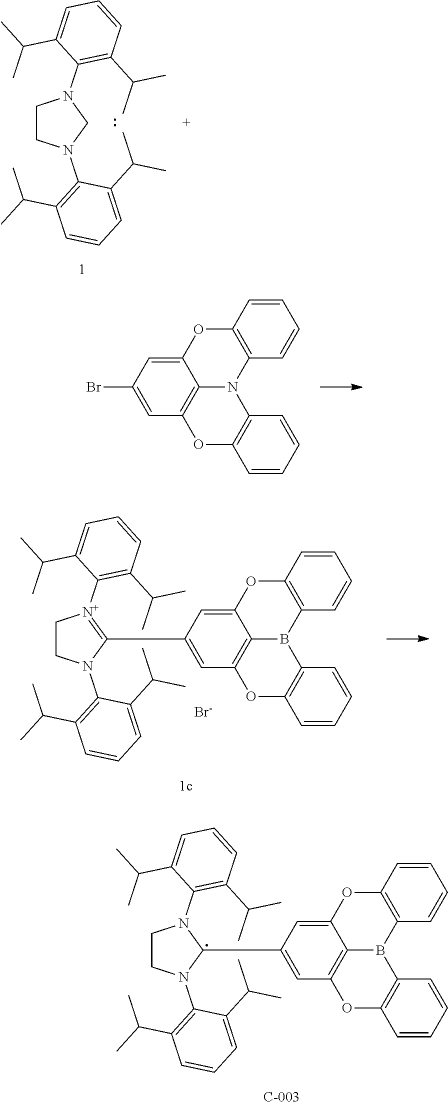

### Synthesis of Compound 1c:

Carbene 1 (0.031 g, 0.08 mmol), 4-bromo-dioxotriphenylamine (0.029 g, 0.09 mmol), and Ni(cod)₂ (1.1 mg, 0.004 mmol, 5 mol %) were added to a dry double-necked flask. After adding 8 mL of dry o-xylene under N₂ atmosphere, the resulting mixture was stirred at room temperature for 10 min, heated to reflux, then stirred for 4 h. After cooling down to room temperature, the organic phases were concentrated, then the resulting sample was purified by column chromatography (eluent: dichloromethane:ethanol=10:1) to yield 0.029 g (50% yield) of compound 1c (yellow solid).

### Synthesis of Azacarbene Carbon Free Radical C-003:

Compound 1c (0.014 mg, 0.02 mmol) and KC8 (0.003 g, 0.02 mmol) were added to a dry schlenck tube. After adding 15 mL of dry-cooled tetrahydrofuran under N₂ atmosphere, the resulting mixture was stirred at −78° C. for 1 h, then the result was heated to room temperature and stirred for 2 h. After the reaction was completed, the result was filtrated, then the filtrate was concentrated and dried to yield 0.011 g (90% yield) of azacarbene carbon free radical C-003 (solid).

### Synthesis Example 4: Synthesis of Azacarbene Carbon Free Radical C-004

The synthetic route of the azacarbene carbon free radical C-004 is as follows:

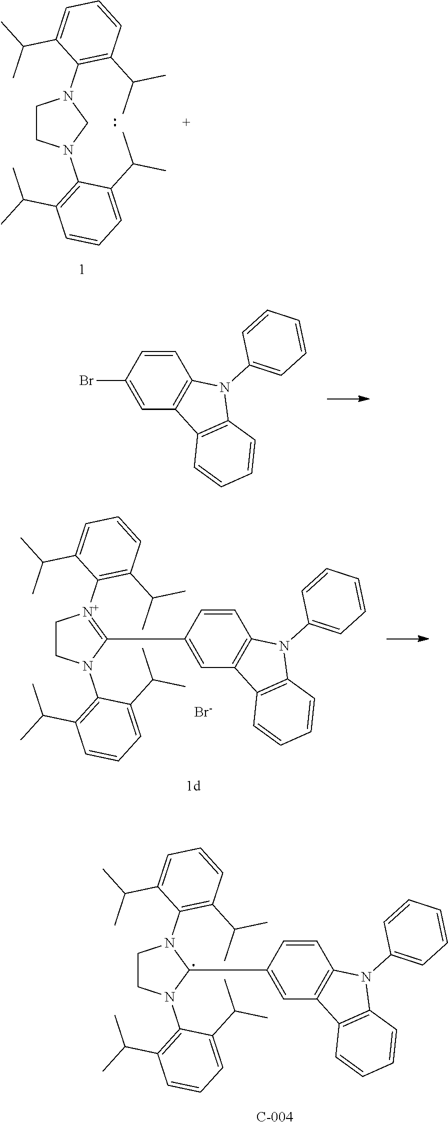

### Synthesis of Compound 1d:

Carbene 1 (0.031 g, 0.08 mmol), 3-bromo-9-phenylcarbazole (0.028 g, 0.09 mmol), and Ni(cod)₂ (1.1 mg, 0.004 mmol, 5 mol %) were added to a dry double-necked flask. After adding 8 mL of dry o-xylene under N₂ atmosphere, the resulting mixture was stirred at room temperature for 10 min, heated to reflux, then stirred for 4 h. After cooling down to room temperature, the organic phases were concentrated, then the resulting sample was purified by column chromatography (eluent: dichloromethane:ethanol=10:1) to yield 0.037 g (65% yield) of compound 1d (yellow solid).

### Synthesis of Azacarbene Carbon Free Radical C-004:

Compound 1d (0.014 mg, 0.02 mmol) and KC8 (0.003 g, 0.02 mmol) were added to a dry schlenck tube. After adding 15 mL of dry-cooled tetrahydrofuran under N₂ atmosphere, the resulting mixture was stirred at −78° C. for 1 h, then the result was heated to room temperature and stirred for 2 h. After the reaction was completed, the result was filtrated, then the filtrate was concentrated and dried to yield 0.011 g (90% yield) of azacarbene carbon free radical C-004 (solid).

### Synthesis Example 5: Synthesis of Azacarbene Carbon Free Radical C-005

The synthetic route for the azacarbene carbon free radical C-005 is as follows:

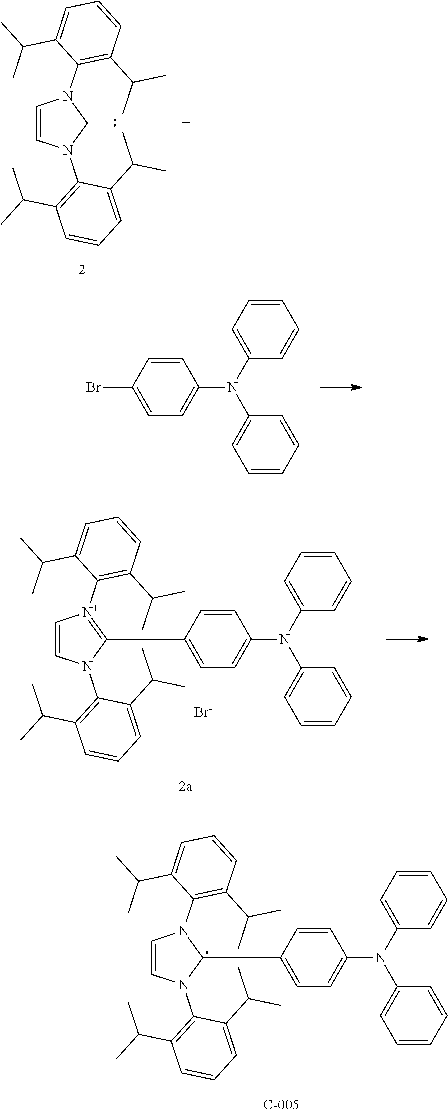

### Synthesis of Compound 2a:

Carbene 2 (0.031 g, 0.08 mmol), 4-bromotriphenylamine (0.029 g, 0.09 mmol), and Ni(cod)₂ (1.1 mg, 0.004 mmol, 5 mol %) were added to a dry double-necked flask. After adding 8 mL of dry o-xylene under N₂ atmosphere, the resulting mixture was stirred at room temperature for 10 min, heated to reflux, then stirred for 4 h. After cooling down to room temperature, the organic phases were concentrated, then the resulting sample was purified by column chromatography (eluent: dichloromethane:ethanol=10:1) to yield 0.034 g (60% yield) of compound 2a (yellow solid).

### Synthesis of Azacarbene Carbon Free Radical C-005:

Compound 2a (0.014 mg, 0.02 mmol) and KC8 (0.003 g, 0.02 mmol) were added to a dry schlenck tube. After adding 15 mL of dry-cooled tetrahydrofuran under N₂ atmosphere, the resulting mixture was stirred at −78° C. for 1 h, then the result was heated to room temperature and stirred for 2 h. After the reaction was completed, the result was filtrated, then the filtrate was concentrated and dried to yield 0.011 g (90% yield) of azacarbene carbon free radical C-005 (solid).

### Synthesis Example 6: Synthesis of Azacarbene Carbon Free Radical C-006

The synthetic route of the azacarbene carbon free radical C-006 is as follows:

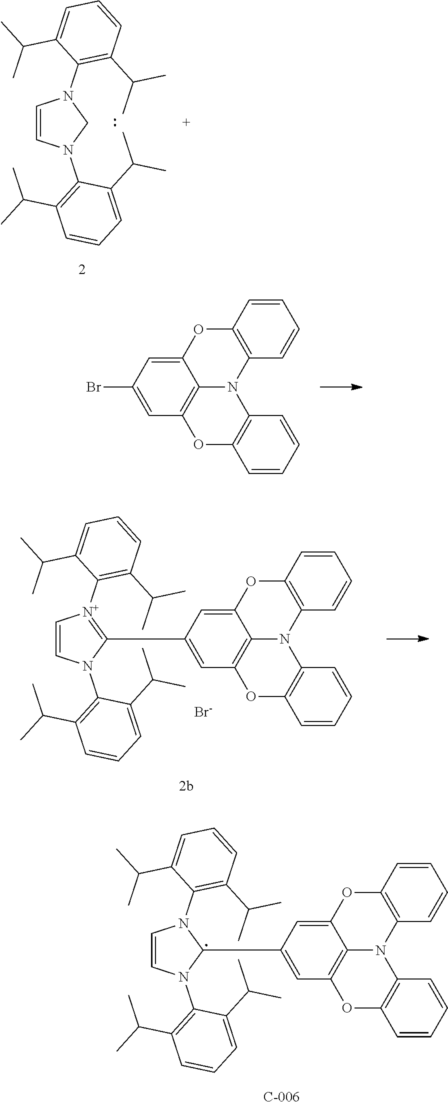

### Synthesis of Compound 2b:

Carbene 2 (0.031 g, 0.08 mmol), 4-bromo-dioxotriphenylamine (0.032 g, 0.09 mmol), and Ni(cod)₂ (1.1 mg, 0.004 mmol, 5 mol %) were added to a dry double-necked flask. After adding 8 mL of dry o-xylene under N₂ atmosphere, the resulting mixture was stirred at room temperature for 10 min, heated to reflux, then stirred for 4 h. After cooling down to room temperature, the organic phases were concentrated, then the resulting sample was purified by column chromatography (eluent: dichloromethane:ethanol=10:1) to yield 0.033 g (55% yield) of compound 2b (yellow solid).

### Synthesis of Azacarbene Carbon Free Radical C-006:

Compound 2b (0.015 mg, 0.02 mmol) and KC8 (0.003 g, 0.02 mmol) were added to a dry schlenck tube. After adding 15 mL of dry-cooled tetrahydrofuran under N₂ atmosphere, the resulting mixture was stirred at −78° C. for 1 h, then the result was heated to room temperature and stirred for 2 h. After the reaction was completed, the result was filtrated, then the filtrate was concentrated and dried to yield 0.012 g (90% yield) of azacarbene carbon free radical C-006 (solid).

### Synthesis Example 7: Synthesis of Azacarbene Carbon Free Radical C-007

The synthetic route of the azacarbene carbon free radical C-007 is as follows:

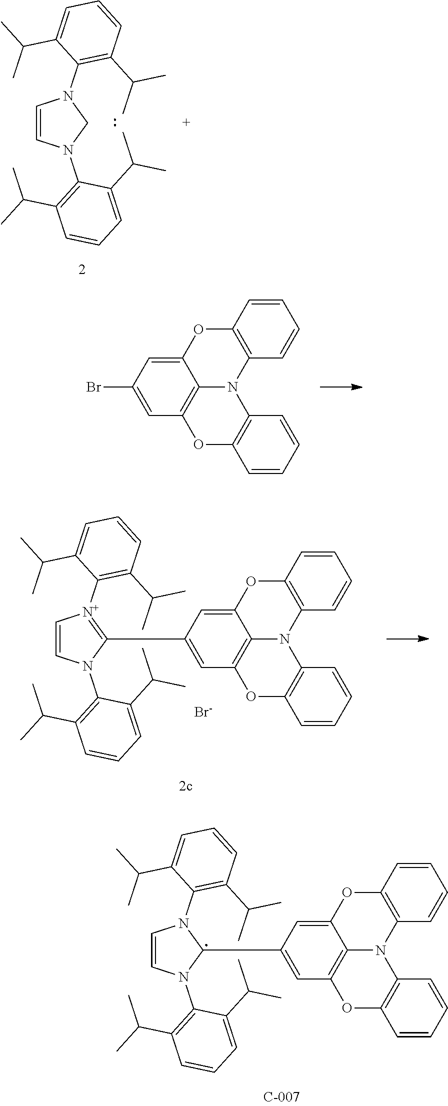

### Synthesis of Compound 2c:

Carbene 2 (0.031 g, 0.08 mmol), 4-bromo-dioxotriphenylboron (0.029 g, 0.09 mmol), and Ni(cod)₂ (1.1 mg, 0.004 mmol, 5 mol %) were added to a dry double-necked flask. After adding 8 mL of dry o-xylene under N₂ atmosphere, the resulting mixture was stirred at room temperature for 10 min, heated to reflux, then stirred for 4 h. After cooling down to room temperature, the organic phases were concentrated, then the resulting sample was purified by column chromatography (eluent: dichloromethane:ethanol=10:1) to yield 0.029 g (50% yield) of compound 2c (yellow solid).

### Synthesis of Azacarbene Carbon Free Radical C-007:

Compound 2c (0.014 mg, 0.02 mmol) and KC8 (0.003 g, 0.02 mmol) were added to a dry schlenck tube. After adding 15 mL of dry-cooled tetrahydrofuran under N₂ atmosphere, the resulting mixture was stirred at −78° C. for 1 h, then the result was heated to room temperature and stirred for 2 h. After the reaction was completed, the result was filtrated, then the filtrate was concentrated and dried to yield 0.011 g (90% yield) of azacarbene carbon free radical C-007 (solid).

### Synthesis Example 8: Synthesis of Azacarbene Carbon Free Radical C-008

The synthetic route of the azacarbene carbon free radical C-008 is as follows:

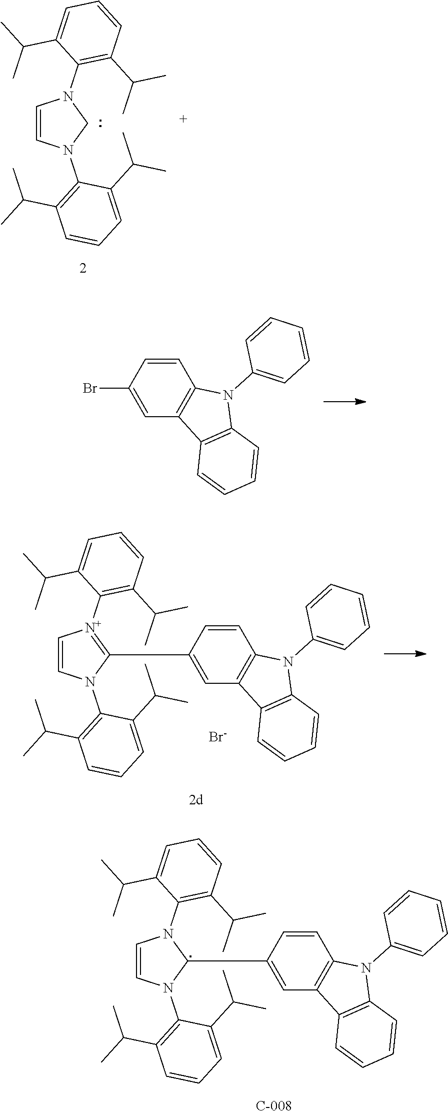

### Synthesis of Compound 2d:

Carbene 2 (0.031 g, 0.08 mmol), 3-bromo-9-phenylcarbazole (0.028 g, 0.09 mmol), and Ni(cod)₂ (1.1 mg, 0.004 mmol, 5 mol %) were added to a dry double-necked flask. After adding 8 mL of dry o-xylene under N₂ atmosphere, the resulting mixture was stirred at room temperature for 10 min, heated to reflux, then stirred for 4 h. After cooling down to room temperature, the organic phases were concentrated, then the resulting sample was purified by column chromatography (eluent: dichloromethane:ethanol=10:1) to yield 0.037 g (65% yield) of compound 2d (yellow solid).

### Synthesis of Azacarbene Carbon Free Radical C-008:

Compound 2d (0.014 mg, 0.02 mmol) and KC8 (0.003 g, 0.02 mmol) were added to a dry schlenck tube. After adding 15 mL of dry-cooled tetrahydrofuran under N₂ atmosphere, the resulting mixture was stirred at −78° C. for 1 h, then the result was heated to room temperature and stirred for 2 h. After the reaction was completed, the result was filtrated, then the filtrate was concentrated and dried to yield 0.011 g (90% yield) of azacarbene carbon free radical C-008 (solid).

### Synthesis Example 9: Synthesis of Azacarbene Carbon Free Radical C-009

The synthetic route of the azacarbene carbon free radical C-009 is as follows:

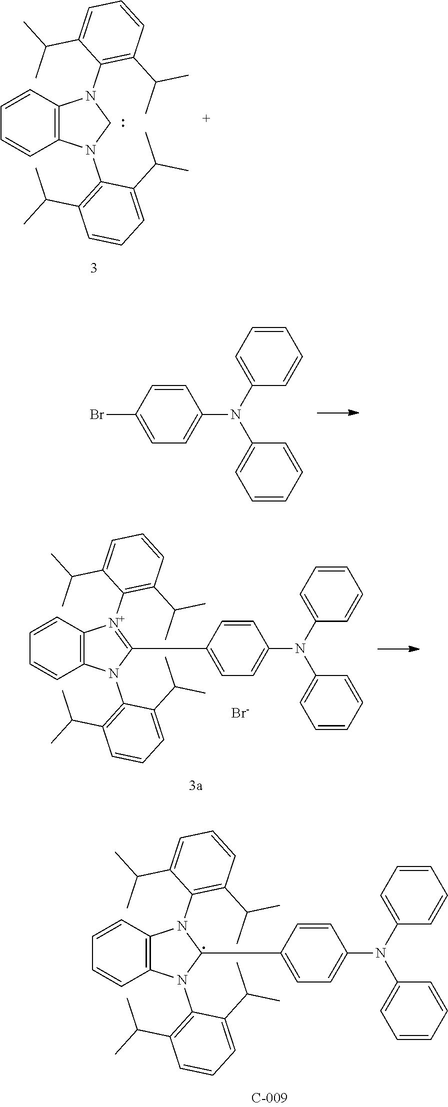

### Synthesis of Compound 3a:

Carbene 3 (0.035 g, 0.08 mmol), 4-bromotriphenylamine (0.029 g, 0.09 mmol), and Ni(cod)₂ (1.1 mg, 0.004 mmol, 5 mol %) were added to a dry double-necked flask. After adding 8 mL of dry o-xylene under N₂ atmosphere, the resulting mixture was stirred for 10 min at room temperature, heated to reflux, then stirred for 4 h. After cooling down to room temperature, the organic phases were concentrated, then the resulting sample was purified by column chromatography (eluent: dichloromethane:ethanol=10:1) to yield 0.033 g (55% yield) of compound 3a (yellow solid).

### Synthesis of Azacarbene Carbon Free Radical C-009:

Compound 3a (0.015 mg, 0.02 mmol) and KC8 (0.003 g, 0.02 mmol) were added to a dry schlenck tube. After adding 15 mL of dry-cooled tetrahydrofuran under N₂ atmosphere, the resulting mixture was stirred at −78° C. for 1 h, then the result was heated to room temperature and stirred for 2 h. After the reaction was completed, the result was filtrated, then the filtrate was concentrated and dried to yield 0.011 g (90% yield) of azacarbene carbon free radical C-009 (solid).

### Synthesis Example 10: Synthesis of Azacarbene Carbon Free Radical C-010

The synthetic route of the azacarbene carbon free radical C-010 is as follows:

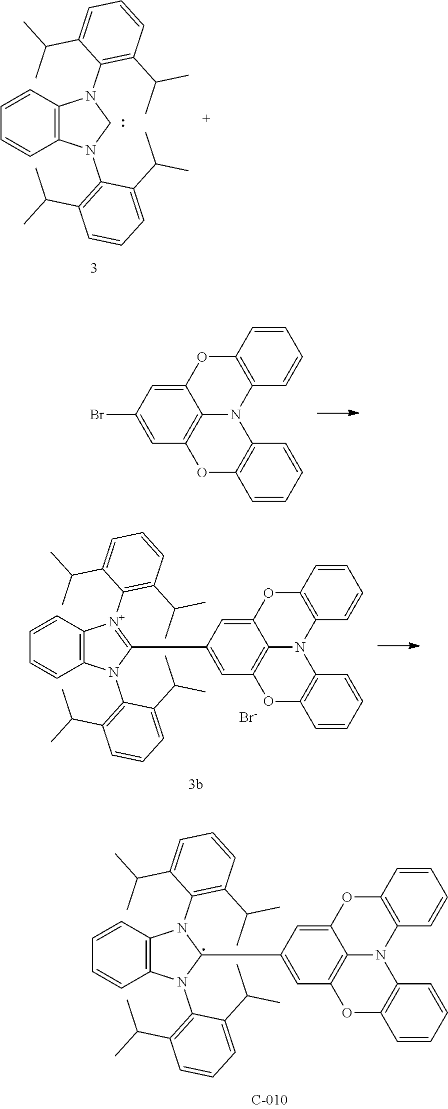

Synthesis of compound 3b:

Carbene 3 (0.035 g, 0.08 mmol), 4-bromo-dioxotriphenylamine (0.032 g, 0.09 mmol), and Ni(cod)₂ (1.1 mg, 0.004 mmol, 5 mol %) were added to a dry double-necked flask. After adding 8 mL of dry o-xylene under N₂ atmosphere, the resulting mixture was stirred at room temperature for 10 min, heated to reflux, then stirred for 4 h. After cooling down to room temperature, the organic phases were concentrated, then the resulting sample was purified by column chromatography (eluent: dichloromethane:ethanol=10:1) to yield 0.038 g (60% yield) of compound 3b (yellow solid).

### Synthesis of Azacarbene Carbon Free Radical C-010:

Compound 3b (0.016 mg, 0.02 mmol) and KCs (0.003 g, 0.02 mmol) were added to a dry schlenck tube. After adding 15 mL of dry-cooled tetrahydrofuran under N₂ atmosphere, the resulting mixture was stirred at −78° C. for 1 h, then the result was heated to room temperature and stirred for 2 h. After the reaction was completed, the result was filtrated, then the filtrate was concentrated and dried to yield 0.012 g (90% yield) of azacarbene carbon free radical C-010 (solid).

### Synthesis Example 11: Synthesis of Azacarbene Carbon Free Radical C-011

The synthetic route of the azacarbene carbon free radical C-011 is as follows:

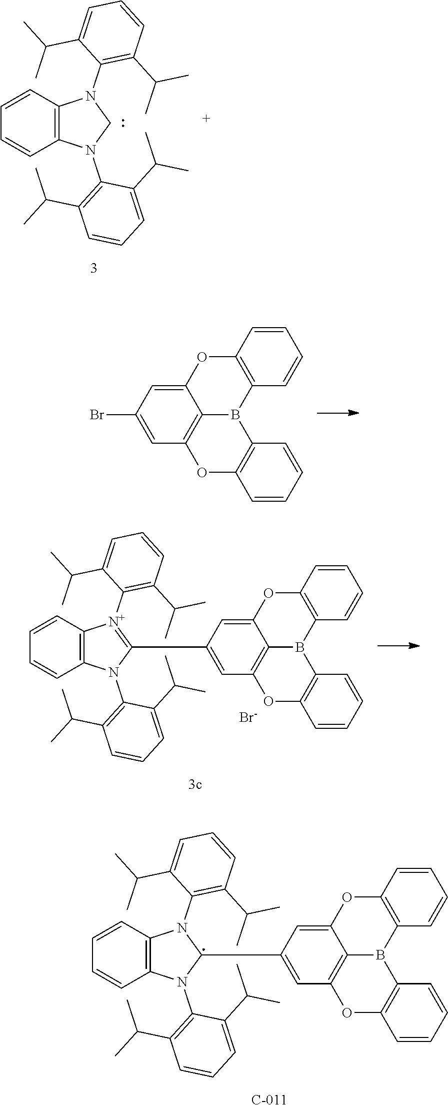

### Synthesis of Compound 3c:

Carbene 3 (0.035 g, 0.08 mmol), 4-bromo-dioxotriphenylamine (0.029 g, 0.09 mmol), and Ni(cod)₂ (1.1 mg, 0.004 mmol, 5 mol %) were added to a dry double-necked flask. After adding 8 mL of dry o-xylene under N₂ atmosphere, the resulting mixture was stirred at room temperature for 10 min, heated to reflux, then stirred for 4 h. After cooling down to room temperature, the organic phases were concentrated, then the resulting sample was purified by column chromatography (eluent: dichloromethane:ethanol=10:1) to yield 0.034 g (55% yield) of compound 3c (yellow solid).

### Synthesis of Azacarbene Carbon Free Radical C-011:

Compound 3c (0.015 mg, 0.02 mmol) and KC8 (0.003 g, 0.02 mmol) were added to a dry schlenck tube. After adding 15 mL of dry-cooled tetrahydrofuran under N₂ atmosphere, the resulting mixture was stirred at −78° C. for 1 h, then the result was heated to room temperature and stirred for 2 h. After the reaction was completed, the result was filtrated, then the filtrate was concentrated and dried to yield 0.012 g (90% yield) of azacarbene carbon free radical C-011 (solid).

### Synthesis Example 12: Synthesis of Azacarbene Carbon Free Radical C-012

The synthetic route of the azacarbene carbon free radical C-012 is as follows:

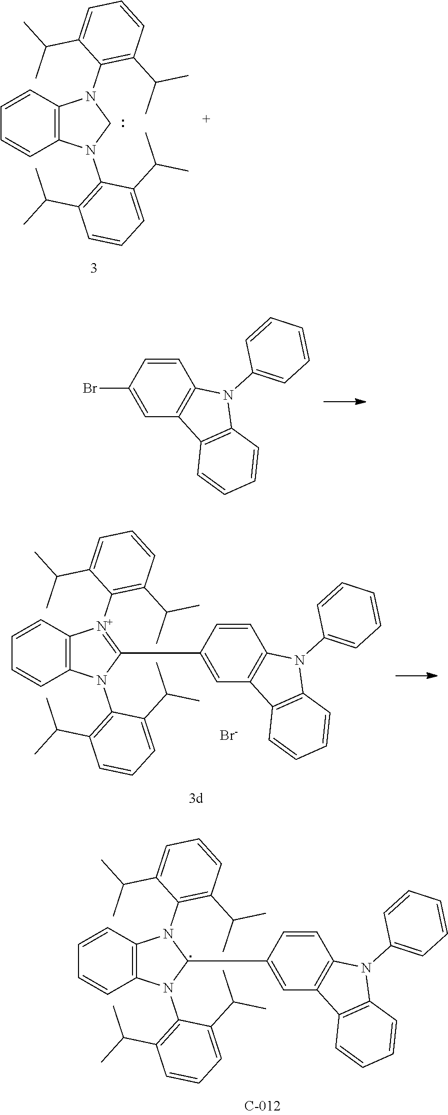

### Synthesis of Compound 3d:

Carbene 3 (0.035 g, 0.08 mmol), 3-bromo-9-phenylcarbazole (0.028 g, 0.09 mmol), and Ni(cod)₂ (1.1 mg, 0.004 mmol, 5 mol %) were added to a dry double-necked flask. After adding 8 mL of dry o-xylene under N₂ atmosphere, the resulting mixture was stirred at room temperature for 10 min, heated to reflux, then stirred for 4 h. After cooling down to room temperature, the organic phases were concentrated, then the resulting sample was purified by column chromatography (eluent: dichloromethane:ethanol=10:1) to yield 0.039 g (65% yield) of compound 3d (yellow solid).

### Synthesis of Azacarbene Carbon Free Radical C-012:

Compound 3d (0.015 mg, 0.02 mmol) and KC8 (0.003 g, 0.02 mmol) were added to a dry schlenck tube. After adding 15 mL of dry-cooled tetrahydrofuran under N₂ atmosphere, the resulting mixture was stirred at −78° C. for 1 h, then the result was heated to room temperature and stirred for 2 h. After the reaction was completed, the result was filtrated, then the filtrate was concentrated and dried to yield 0.012 g (90% yield) of azacarbene carbon free radical C-012 (solid).

### Mixtures and Formulations

The above-mentioned free radical molecules C-001˜C-012 and TPBi with TPBi: 4 wt % C-00x (x=001-012) were thoroughly ground and mixed in a nitrogen-regulated glove box to obtain a mixture x (x=001-012).

The mixture x (x=001-012) was added to chloroform at a ratio of 10 mg/mL, then heated and stirred thoroughly to dissolve the above mixture to obtain a formulation x (x=001-012). The obtained formulation x can be used to prepare solution-processed organic electronic devices.

### 3. Preparation and Characterization of OLED Devices

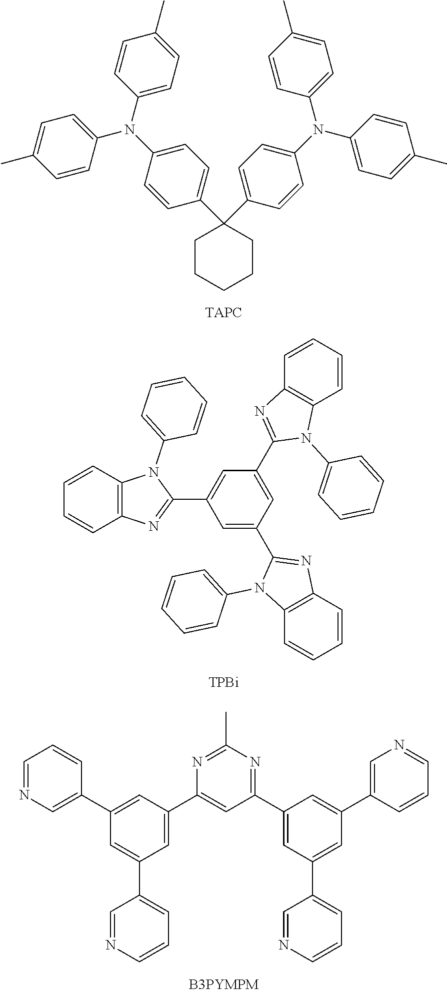

The preparation process of the OLEDs comprising the free radical molecule as an emitter will be described in detail with reference to specified examples below. The structure of the OLED is as follows: ITO/MoO₃ (3 nm)/TAPC (40 nm)/TPBi: 4 wt % Emitter (30 nm)/B3PYMPM (40 nm)/LiF (0.8 nm)/Al (100 nm).

a. Cleaning of the ITO (Indium Tin Oxide) conductive glass substrate: the substrates are washed with various solvents (such as one or more of chloroform, ketone, or isopropyl alcohol), and then treated with UV and ozone.

b. Evaporation: The resultant ITO substrate was mounted on a vacuum deposition apparatus in high vacuum (1×10⁻⁶ mbar), and MoO₃ was then vacuum-deposited on the anode to form a hole-injection layer having a thickness of 30 nm, then HT (TAPC) was evaporated on the hole-injection layer to form a hole-transport layer having a thickness of 40 nm. Then TPBi and Emitter in two different evaporation sources were deposited at a weight ratio of 96:4 to form a light-emitting layer having a thickness of 30 nm. Subsequently, an electron-transport layer (B3PYMPM) (40 nm) was evaporated, LiF was then deposited on the electron-transport layer to form an electron-injection layer having a thickness of 0.8 nm, and Al was deposited on the electron-injection layer to form a cathode having a thickness of 100 nm.

c. Encapsulation: encapsulating the device in a nitrogen-regulated glove box with UV curable resin.

As shown in Table 1, the specific performance of the device examples were tested; wherein EQE@1000 nits is defined as the external quantum efficiency of the device at a brightness of 1000 nits.

TABLE 1

| OLED Devices | Emitter | EQE@1000 nits | Color |
|---|---|---|---|
| OLED1 | C-005 | 11.5% | Green |
| OLED2 | C-006 | 9.8% | Yellow |

Other free radical molecules as emitters for OLEDs can be prepared and characterized in the same way.

It will be understood that the application of the present disclosure is not limited to the foregoing examples, and may be improved or transformed in accordance with the foregoing description to one of ordinary skill in the art, all these modifications and improvements are within the scope of the present disclosure.

What is claimed is:

1. An azacarbene carbon free radical molecule, comprising a structure of formula (I): 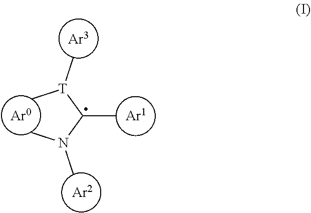
wherein:
T is a single bond, or a carbon atom, or a nitrogen atom, when T is a single bond, Ar³ does not exist;
Ar⁰ is a substituted/unsubstituted single bond or double bond, or an alkane, olefin, aromatic hydrocarbon or heteroaromatic cyclic hydrocarbon system, which is unsubstituted or substituted by one or more Rs;
Ar¹-Ar³ are each independently selected from an aromatic group containing 6 to 60 ring atoms, a heteroaromatic group containing 5 to 60 ring atoms, or a non-aromatic ring system containing 3 to 30 ring atoms, which are unsubstituted or substituted by R;
R, in multiple occurrences, is independently selected from a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, or any combination thereof.

2. The azacarbene carbon free radical molecule according to claim 1, wherein Ar⁰ is selected from a substituted/unsubstituted single bond, a substituted/unsubstituted double bond, a substituted/unsubstituted aromatic group containing 6 to 60 ring atoms, a substituted/unsubstituted heteroaromatic group containing 5 to 60 ring atoms, or a substituted/unsubstituted non-aromatic ring system containing 3 to 30 ring atoms.

3. The azacarbene carbon free radical molecule according to claim 1, wherein Ar¹-Ar³ are each independently selected from the following groups or any combination thereof: 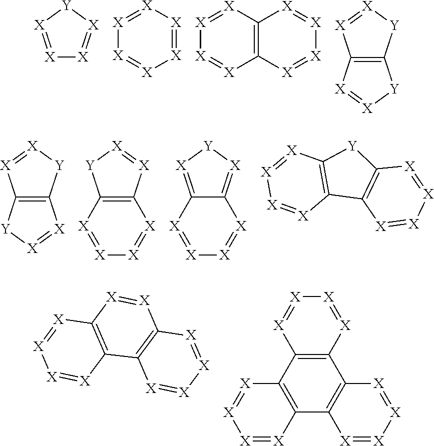
wherein:
Y at each occurrence independently represents CR¹R², NR¹, O, S, SiR¹R², PR¹, P(—O)R¹, S═O, S(═O)², or C═O;
X at each occurrence independently represents CR¹ or N;
each of R¹ and R² at each occurrence is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, or any combination thereof.

4. The azacarbene carbon free radical molecule according to claim 2, wherein Ar¹-Ar³ are each independently selected from the following groups or any combination thereof: 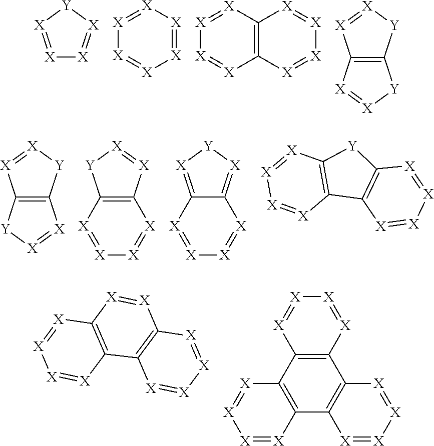
wherein:
Y at each occurrence independently represents CR¹R², NR¹, O, S, SiR¹R², PR¹, P(═O)R¹, S═O, S(═O)₂, or C═O;
X at each occurrence independently represents CR¹ or N;
each of R¹ and R² at each occurrence is independently selected from —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, or any combination thereof.

5. The azacarbene carbon free radical molecule according to claim 1, wherein the azacarbene carbon free radical molecule is selected from one of formulas (II-1)-(II-5): 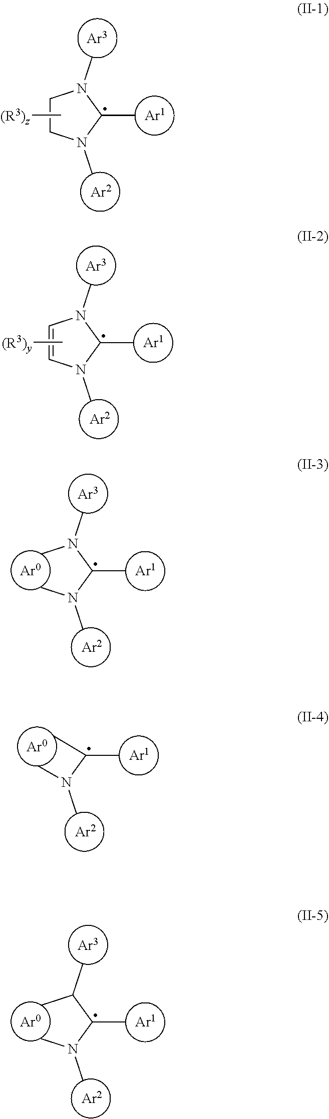
wherein, Ar⁰ is a substituted/unsubstituted single bond or double bond, or an alkane, olefin, aromatic hydrocarbon or heteroaromatic cyclic hydrocarbon system, which is unsubstituted or substituted by one or more Rs; Ar¹-Ar³ are each independently selected from an aromatic group containing 6 to 60 ring atoms, a heteroaromatic group containing 5 to 60 ring atoms, or a non-aromatic ring system containing 3 to 30 ring atoms, which are unsubstituted or substituted by R; y is an integer from 0 to 2; z is an integer from 0 to 4; R³, in multiple occurrences, is independently selected from a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, or any combination thereof.

6. The azacarbene carbon free radical molecule according to claim 2, wherein the azacarbene carbon free radical molecule is selected from one of formulas (II-1)-(II-5): 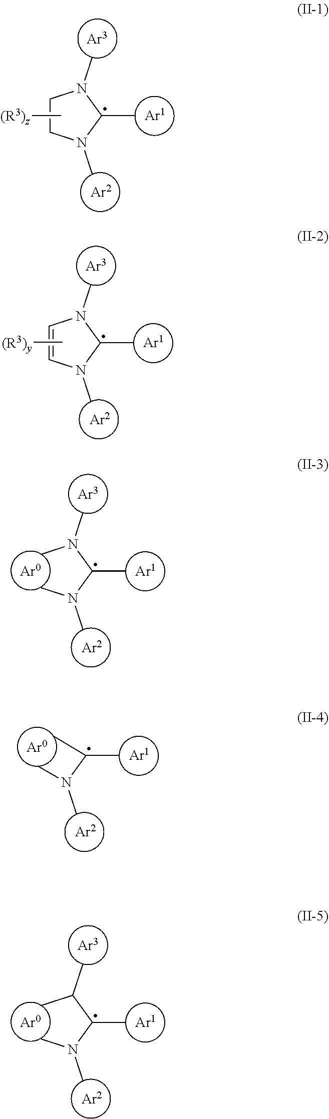
wherein, Ar⁰ is a substituted/unsubstituted single bond or double bond, or an alkane, olefin, aromatic hydrocarbon or heteroaromatic cyclic hydrocarbon system, which is unsubstituted or substituted by one or more Rs; Ar¹-Ar³ are each independently selected from an aromatic group containing 6 to 60 ring atoms, a heteroaromatic group containing 5 to 60 ring atoms, or a non-aromatic ring system containing 3 to 30 ring atoms, which are unsubstituted or substituted by R; y is an integer from 0 to 2; z is an integer from 0 to 4; R³, in multiple occurrences, is independently selected from a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, or any combination thereof.

7. The azacarbene carbon free radical molecule according to claim 3, wherein the azacarbene carbon free radical molecule is selected from one of formulas (II-1)-(II-5): 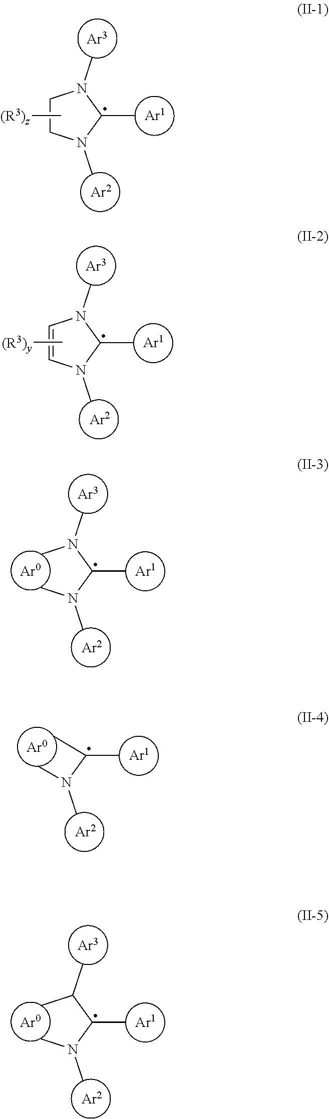
wherein, Ar⁰ is a substituted/unsubstituted single bond or double bond, or an alkane, olefin, aromatic hydrocarbon or heteroaromatic cyclic hydrocarbon system, which is unsubstituted or substituted by one or more Rs; Ar¹-Ar³ are each independently selected from an aromatic group containing 6 to 60 ring atoms, a heteroaromatic group containing 5 to 60 ring atoms, or a non-aromatic ring system containing 3 to 30 ring atoms, which are unsubstituted or substituted by R; y is an integer from 0 to 2; z is an integer from 0 to 4; R³, in multiple occurrences, is independently selected from a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, or any combination thereof.

8. The azacarbene carbon free radical molecule according to claim 4, wherein the azacarbene carbon free radical molecule is selected from one of formulas (II-1)-(II-5): 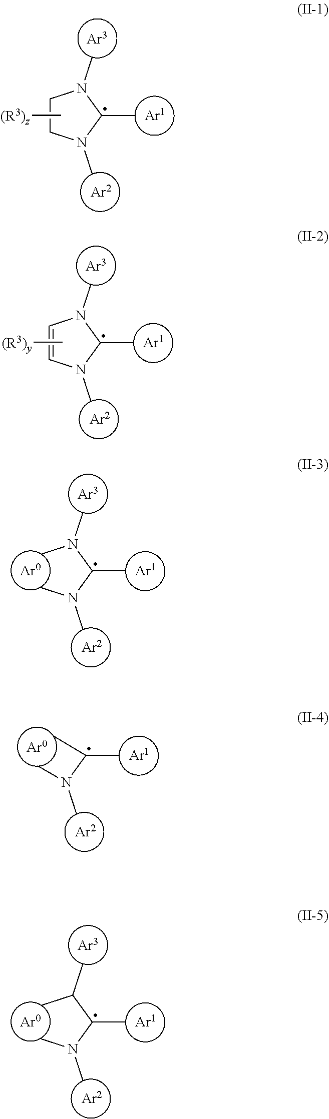
wherein, Ar⁰ is a substituted/unsubstituted single bond or double bond, or an alkane, olefin, aromatic hydrocarbon or heteroaromatic cyclic hydrocarbon system, which is unsubstituted or substituted by one or more Rs; Ar¹-Ar³ are each independently selected from an aromatic group containing 6 to 60 ring atoms, a heteroaromatic group containing 5 to 60 ring atoms, or a non-aromatic ring system containing 3 to 30 ring atoms, which are unsubstituted or substituted by R; y is an integer from 0 to 2; z is an integer from 0 to 4; R³, in multiple occurrences, is independently selected from a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, or any combination thereof.

9. The azacarbene carbon free radical molecule according to claim 1, wherein each Ar¹ is independently selected from one of formulas A1-A20: 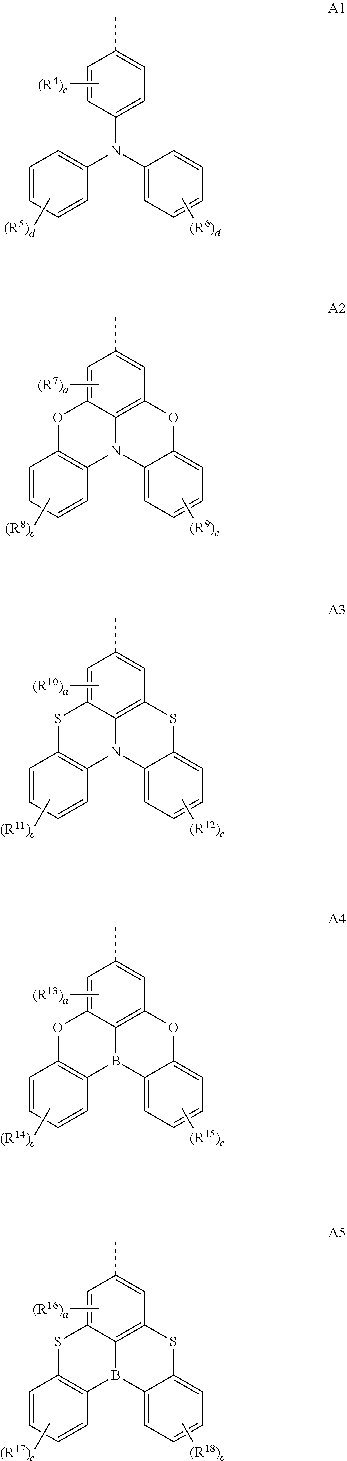 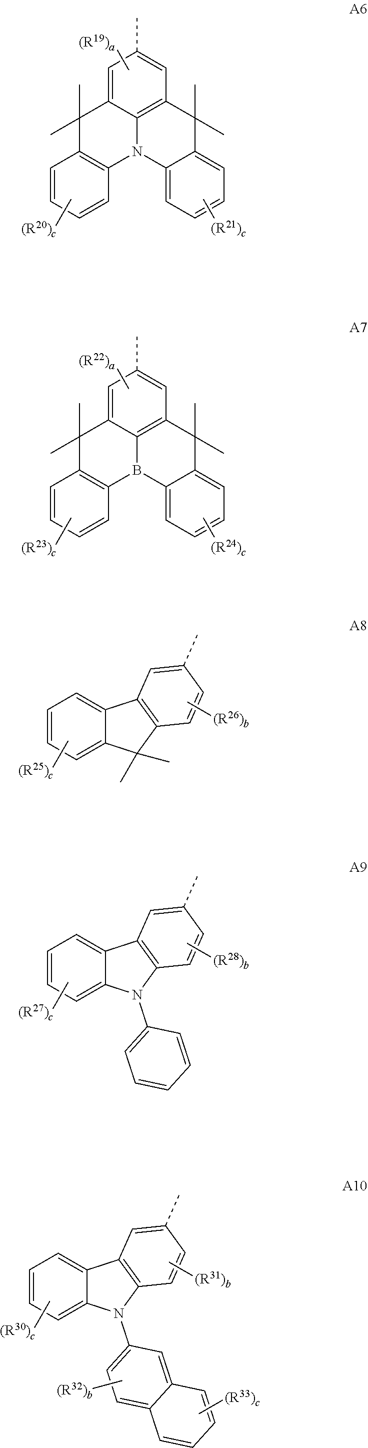 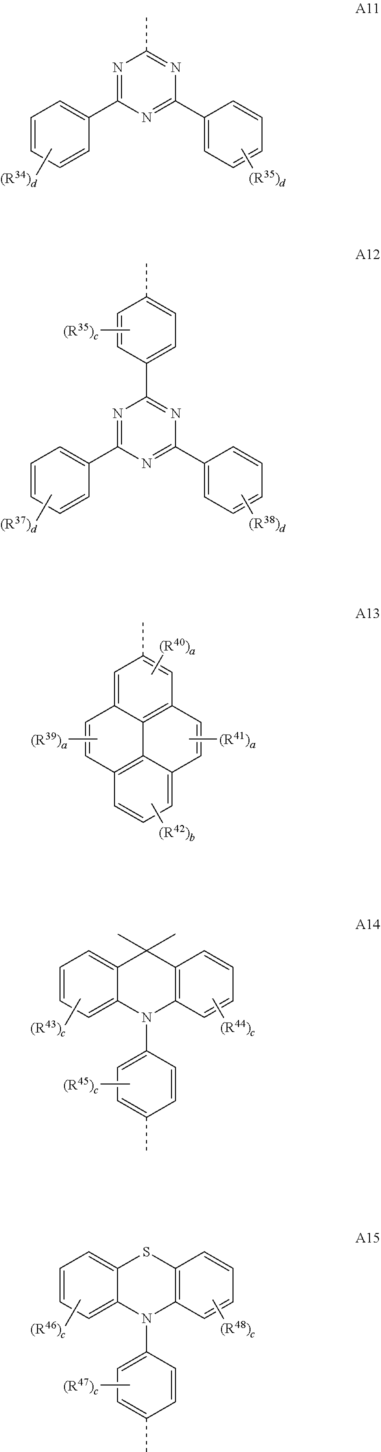 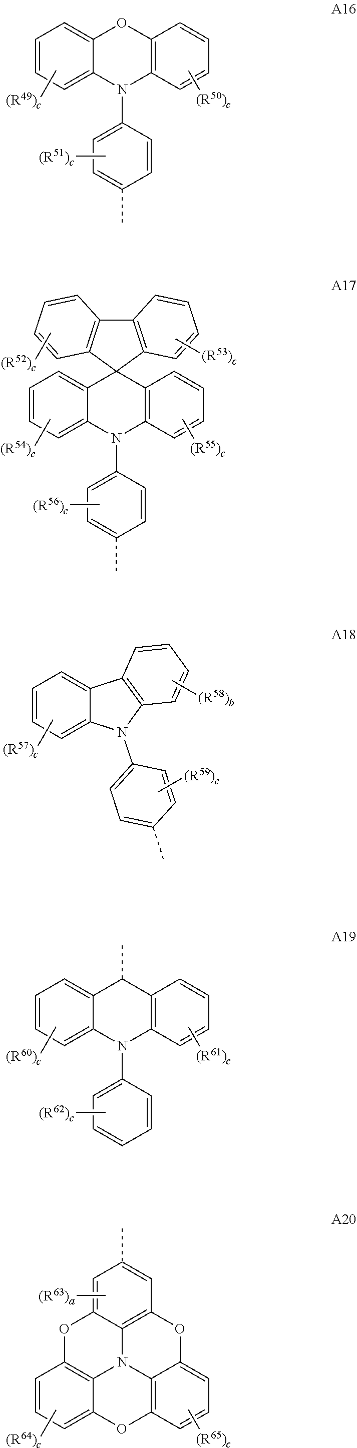
wherein:
a is an integer from 0 to 2; b is an integer from 0 to 3; c is an integer from 0 to 4; d is an integer from 0 to 5;
R⁴-R⁶⁵ are each independently selected from F, Cl, Br, I, D, CN, NO₂, CF₃, B(OR₀)₂, Si(R₀)₃, a linear alkane, an alkane ether, a C₁-C₁₀ alkane sulfide, a C₁-C₁₀ branched alkane, a C₁-C₁₀ cycloalkane, a C₃-C₁₀ alkane ether, a C₃-C₁₀ alkane sulfide group, or a C₆-C₁₀ aryl group;
each dotted line independently represents a bond directly linked to an azacarbene carbon free radical group.

10. The azacarbene carbon free radical molecule according to claim 2, wherein each Ar¹ is independently selected from one of formulas A1-A20: 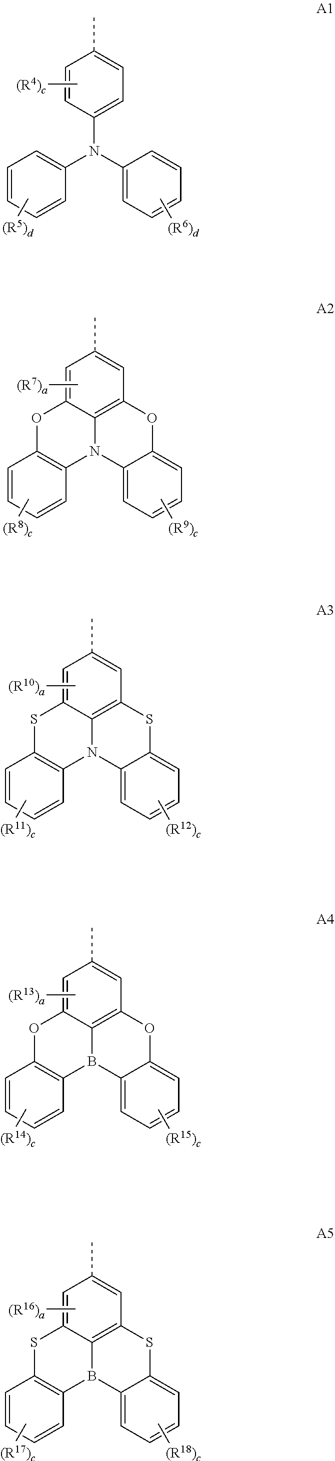 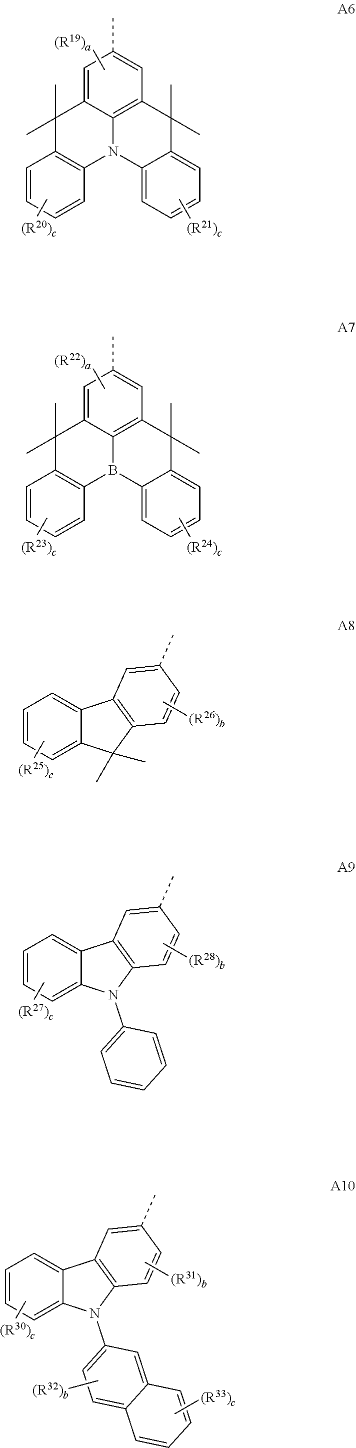 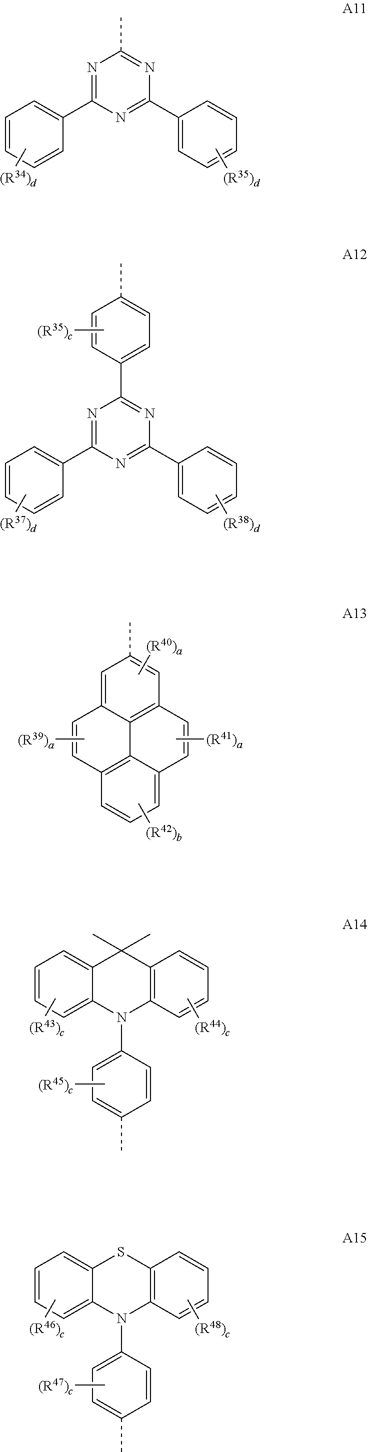 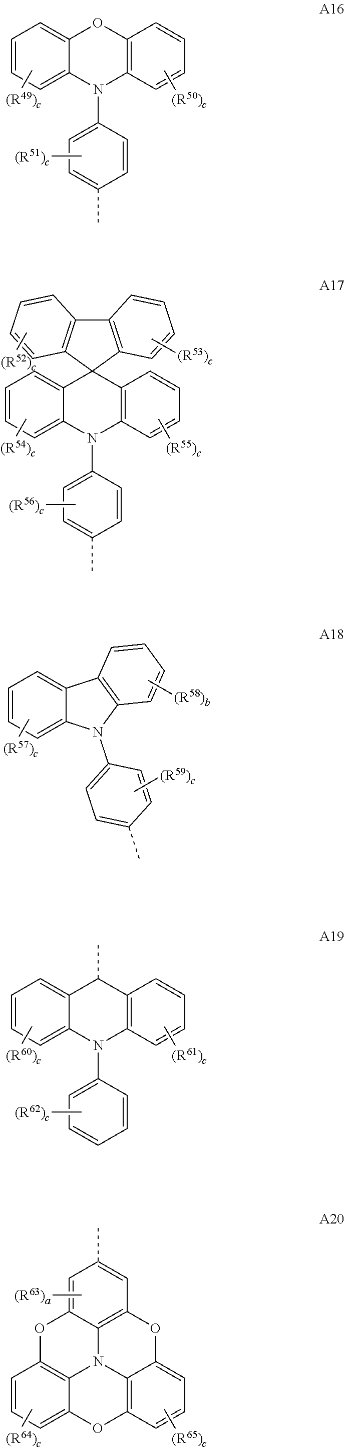
wherein:
a is an integer from 0 to 2; b is an integer from 0 to 3; c is an integer from 0 to 4; d is an integer from 0 to 5;
R⁴-R⁶⁵ are each independently selected from F, Cl, Br, I, D, CN, NO₂, CF₃, B(OR₀)₂, Si(R₀)₃, a linear alkane, an alkane ether, a C₁-C₁₀ alkane sulfide, a C₁-C₁₀ branched alkane, a C₁-C₁₀ cycloalkane, a C₃-C₁₀ alkane ether, a C₃-C₁₀ alkane sulfide group, or a C₆-C₁₀ aryl group;
each dotted line independently represents a bond directly linked to an azacarbene carbon free radical group.

11. The azacarbene carbon free radical molecule according to claim 3, wherein each Ar¹ is independently selected from one of formulas A1-A20: 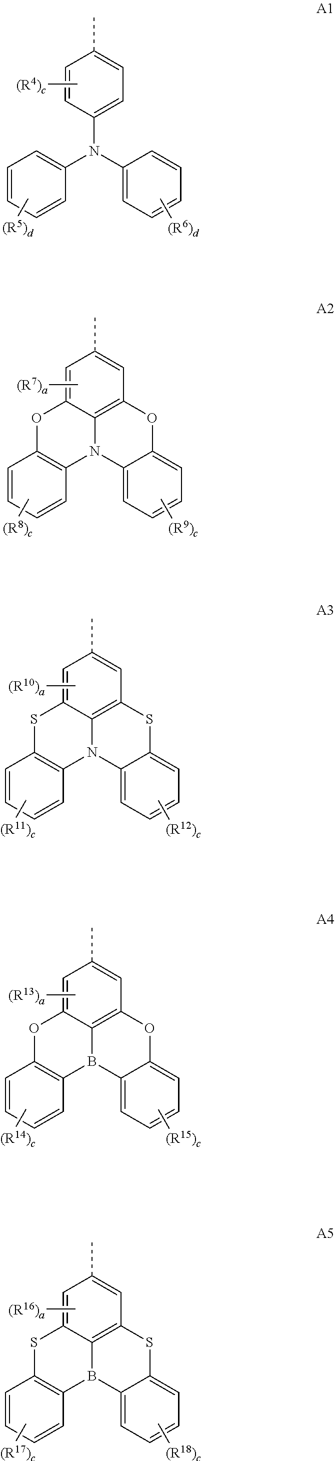 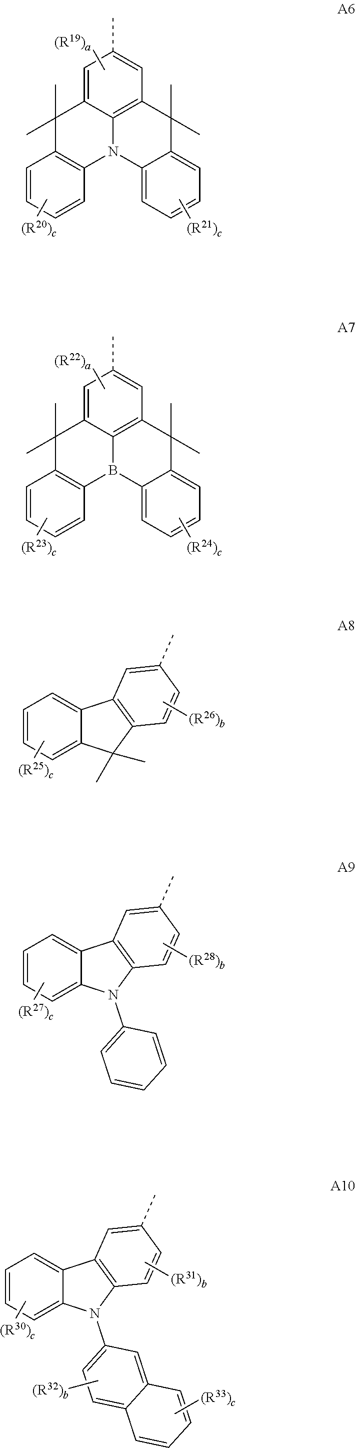 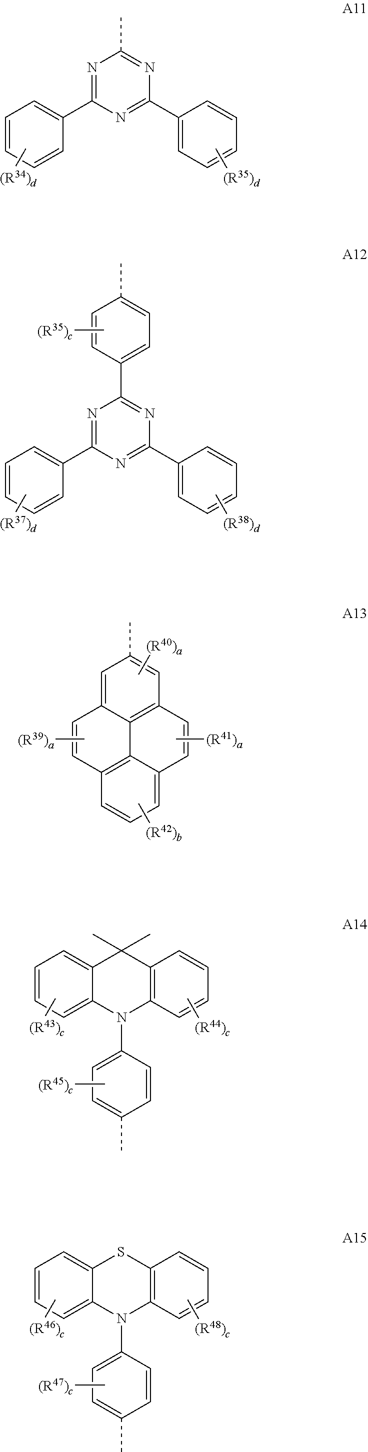 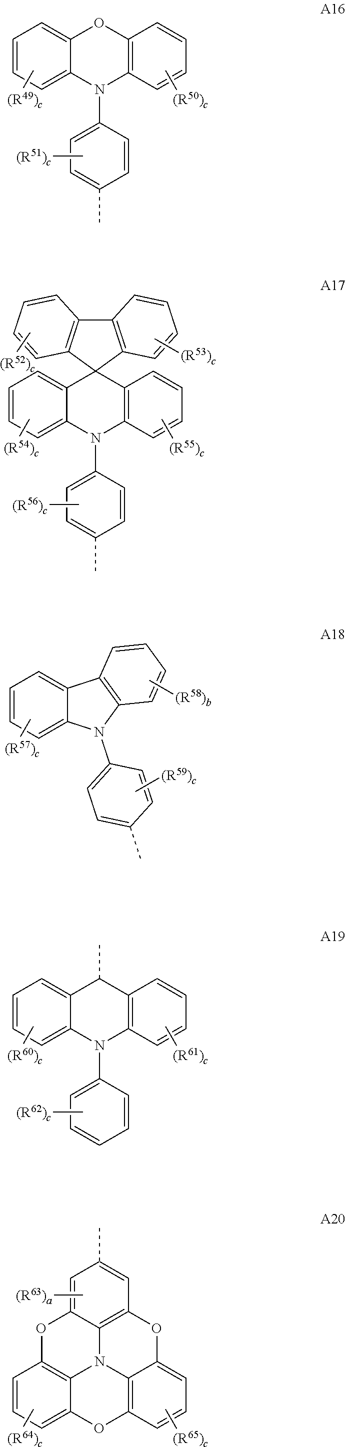
wherein:
a is an integer from 0 to 2; b is an integer from 0 to 3; c is an integer from 0 to 4; d is an integer from 0 to 5;
R⁴-R⁶⁵ are each independently selected from F, Cl, Br, I, D, CN, NO₂, CF₃, B(OR₀)₂, Si(R₀)₃, a linear alkane, an alkane ether, a C₁-C₁₀ alkane sulfide, a C₁-C₁₀ branched alkane, a C₁-C₁₀ cycloalkane, a C₃-C₁₀ alkane ether, a C₃-C₁₀ alkane sulfide group, or a C₆-C₁₀ aryl group;
each dotted line independently represents a bond directly linked to an azacarbene carbon free radical group.

12. The azacarbene carbon free radical molecule according to claim 4, wherein each Ar¹ is independently selected from one of formulas A1-A20: 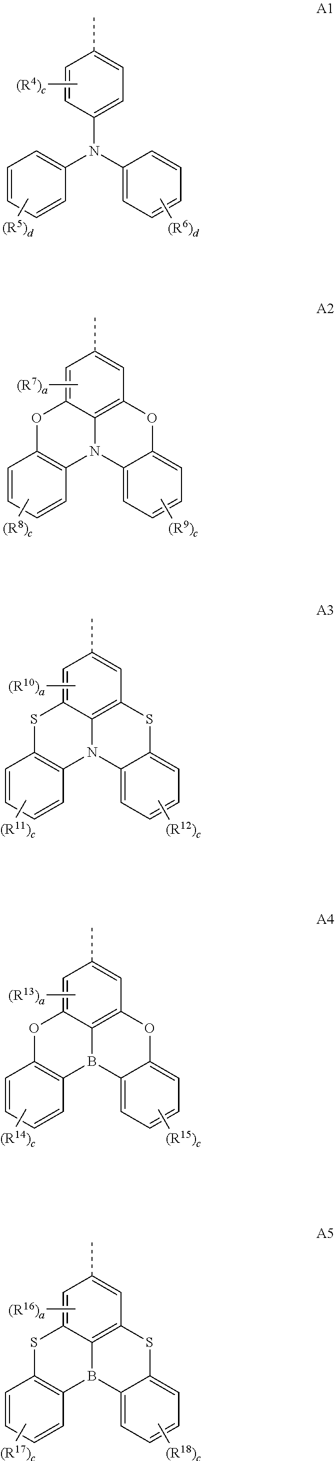 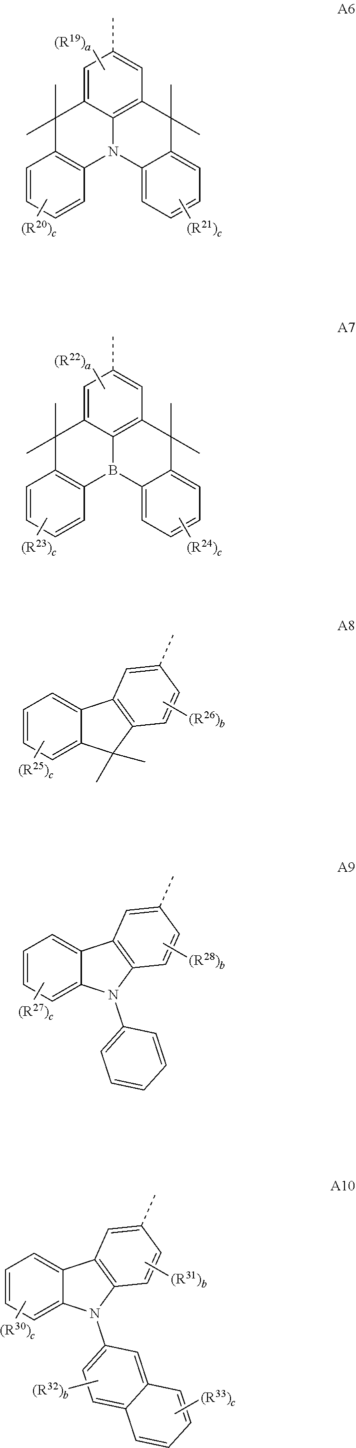 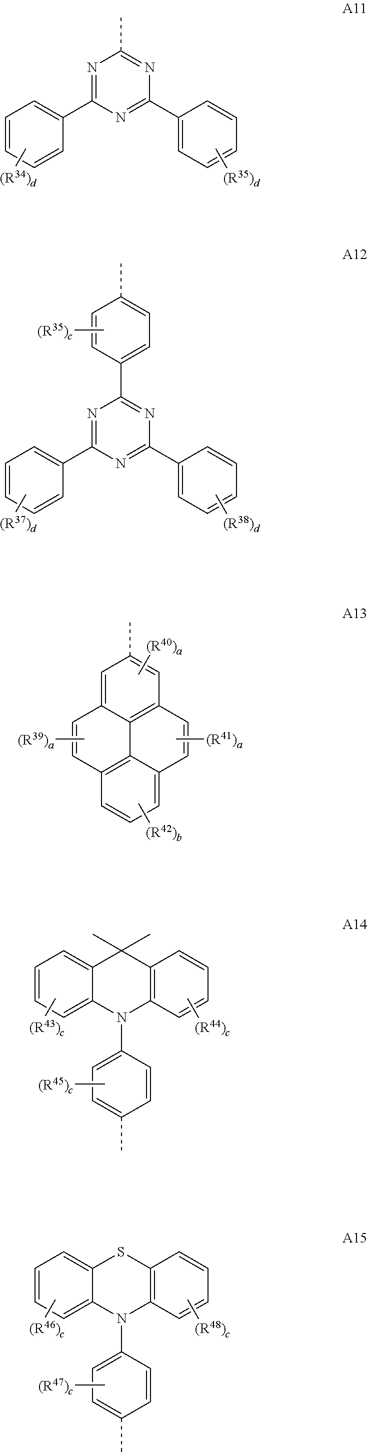 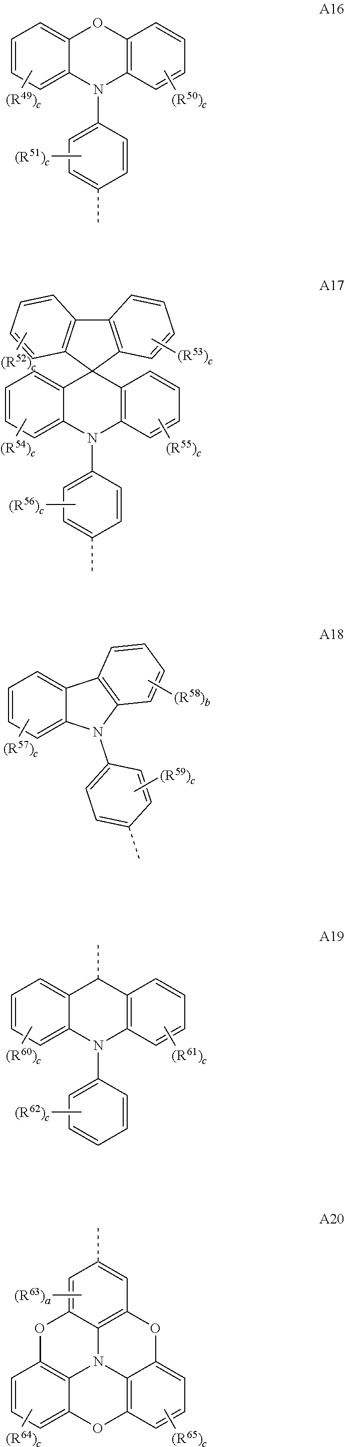
wherein:
a is an integer from 0 to 2; b is an integer from 0 to 3; c is an integer from 0 to 4; d is an integer from 0 to 5;
R⁴-R⁶⁵ are each independently selected from F, Cl, Br, I, D, CN, NO₂, CF₃, B(OR₀)₂, Si(R₀)₃, a linear alkane, an alkane ether, a C₁-C₁₀ alkane sulfide, a C₁-C₁₀ branched alkane, a C₁-C₁₀ cycloalkane, a C₃-C₁₀ alkane ether, a C₃-C₁₀ alkane sulfide group, or a C₆-C₁₀ aryl group;
each dotted line independently represents a bond directly linked to an azacarbene carbon free radical group.

13. The azacarbene carbon free radical molecule according to claim 5, wherein each Ar¹ is independently selected from one of formulas A1-A20: 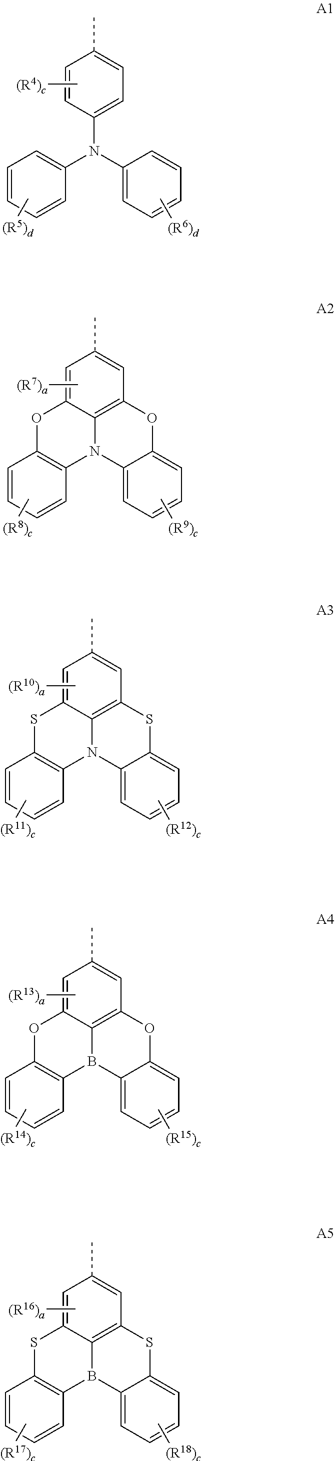 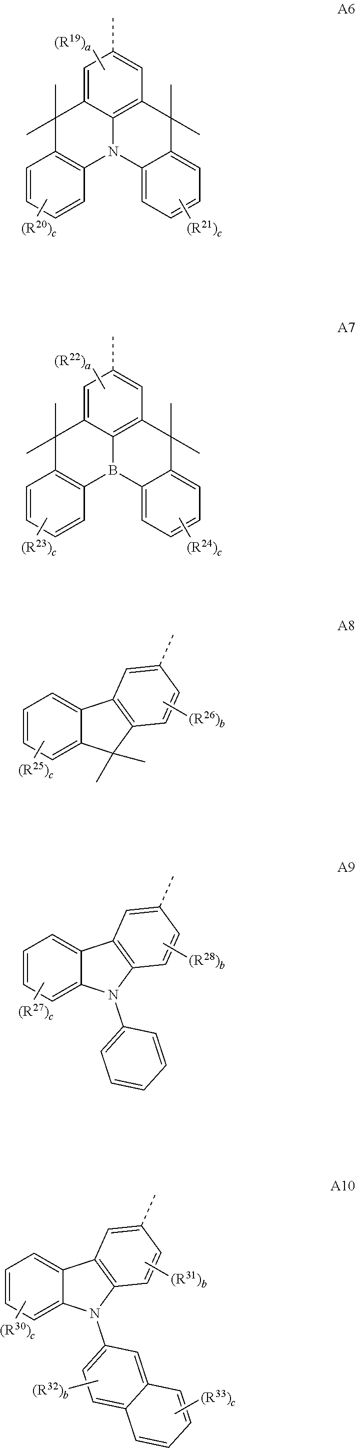 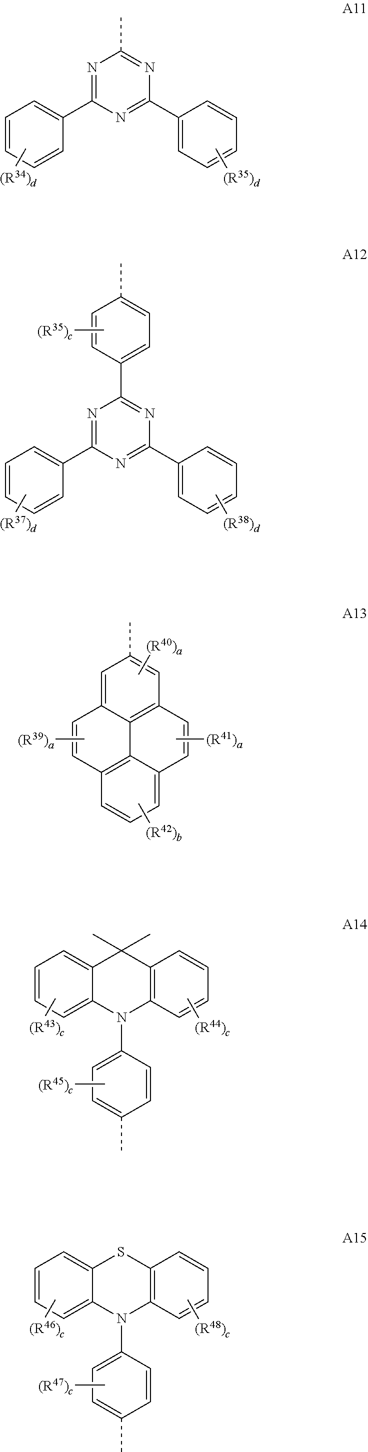 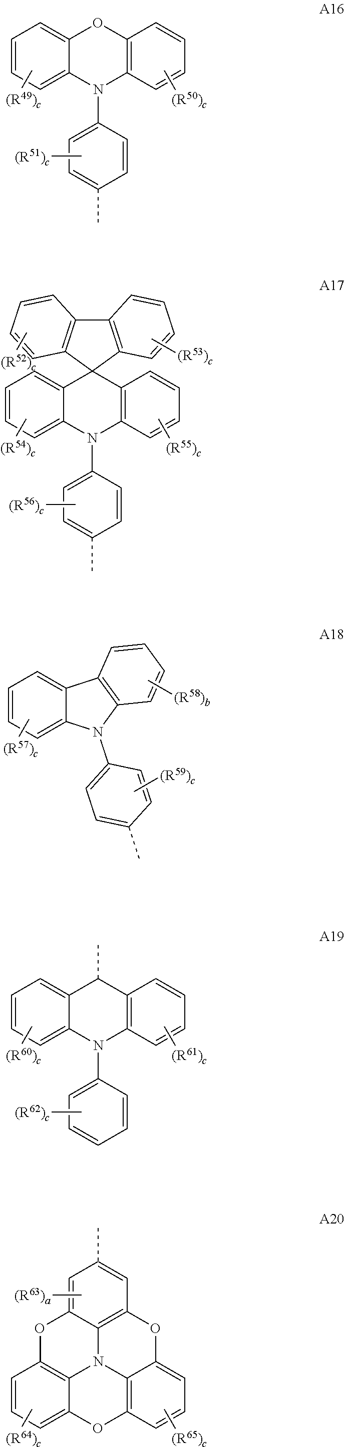
wherein:
a is an integer from 0 to 2; b is an integer from 0 to 3; c is an integer from 0 to 4; d is an integer from 0 to 5;
R⁴-R⁶⁵ are each independently selected from F, Cl, Br, I, D, CN, NO₂, CF₃, B(OR₀)₂, Si(R₀)₃, a linear alkane, an alkane ether, a C₁-C₁₀ alkane sulfide, a C₁-C₁₀ branched alkane, a C₁-C₁₀ cycloalkane, a C₃-C₁₀ alkane ether, a C₃-C₁₀ alkane sulfide group, or a C₆-C₁₀ aryl group;
each dotted line independently represents a bond directly linked to an azacarbene carbon free radical group.

14. The azacarbene carbon free radical molecule according to claim 6, wherein each Ar¹ is independently selected from one of formulas A1-A20: 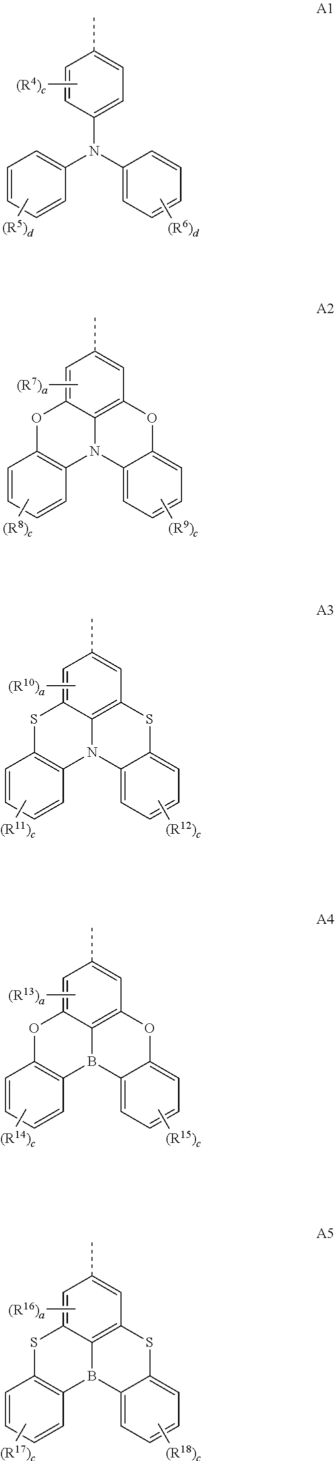 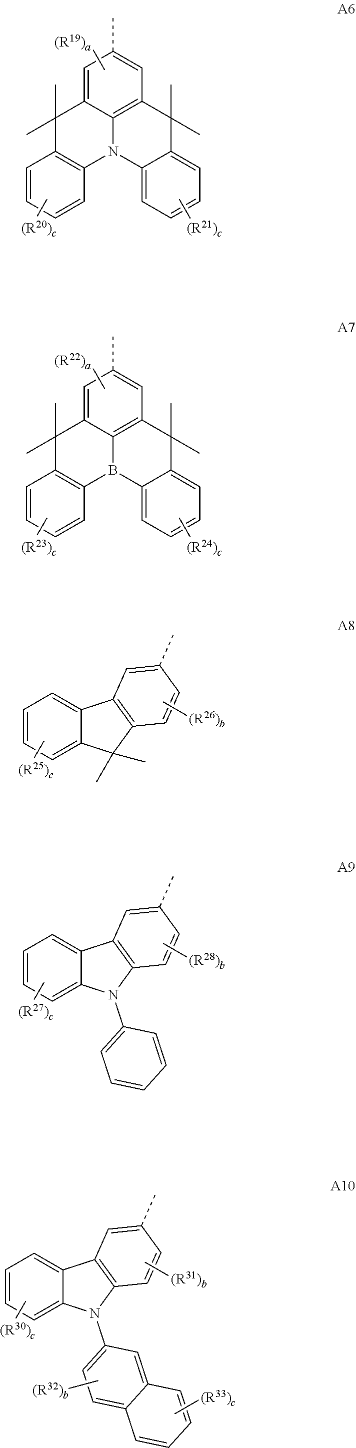 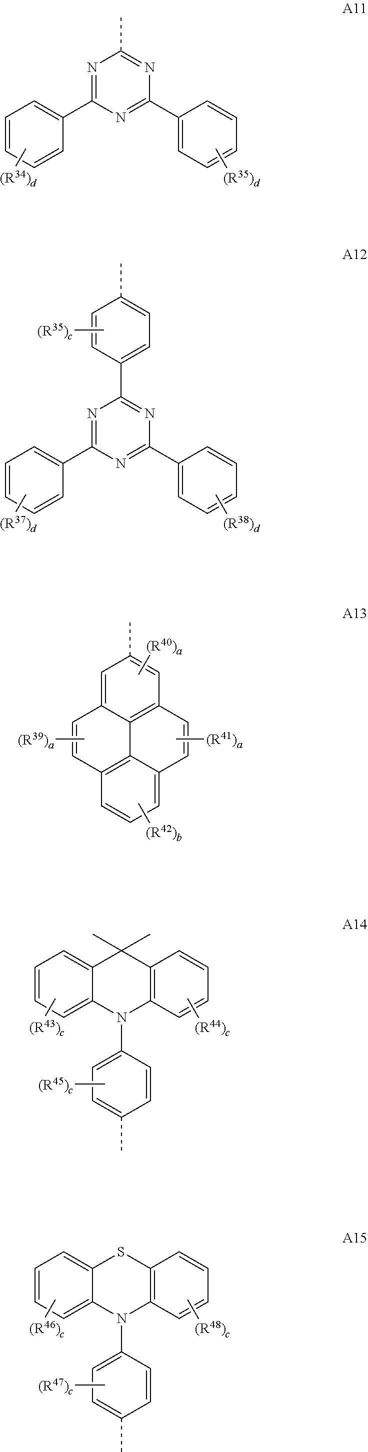 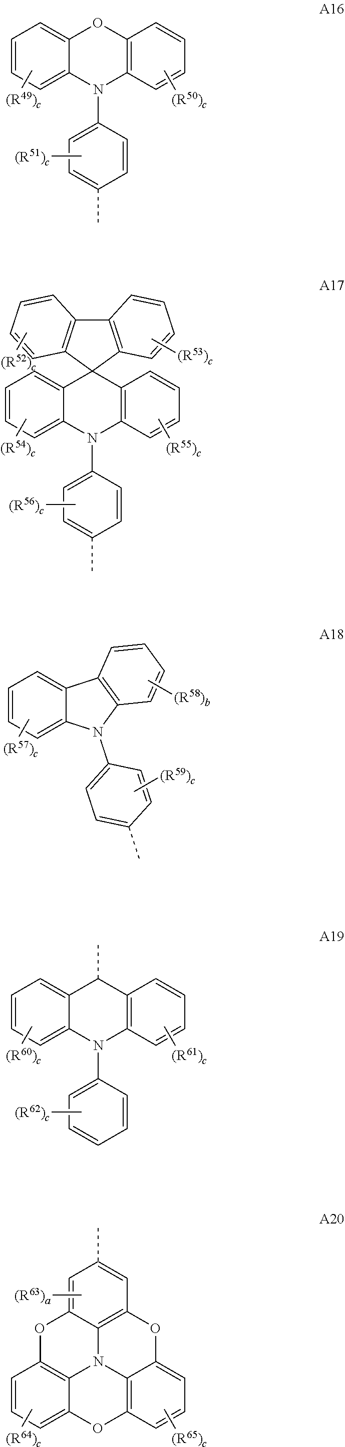
wherein:
a is an integer from 0 to 2; b is an integer from 0 to 3; c is an integer from 0 to 4; d is an integer from 0 to 5;
R⁴-R⁶⁵ are each independently selected from F, Cl, Br, I, D, CN, NO₂, CF₃, B(OR₀)₂, Si(R₀)₃, a linear alkane, an alkane ether, a C₁-C₁₀ alkane sulfide, a C₁-C₁₀ branched alkane, a C₁-C₁₀ cycloalkane, a C₃-C₁₀ alkane ether, a C₃-C₁₀ alkane sulfide group, or a C₆-C₁₀ aryl group;
each dotted line independently represents a bond directly linked to an azacarbene carbon free radical group.

15. The azacarbene carbon free radical molecule according to claim 7, wherein each Ar¹ is independently selected from one of formulas A1-A20: 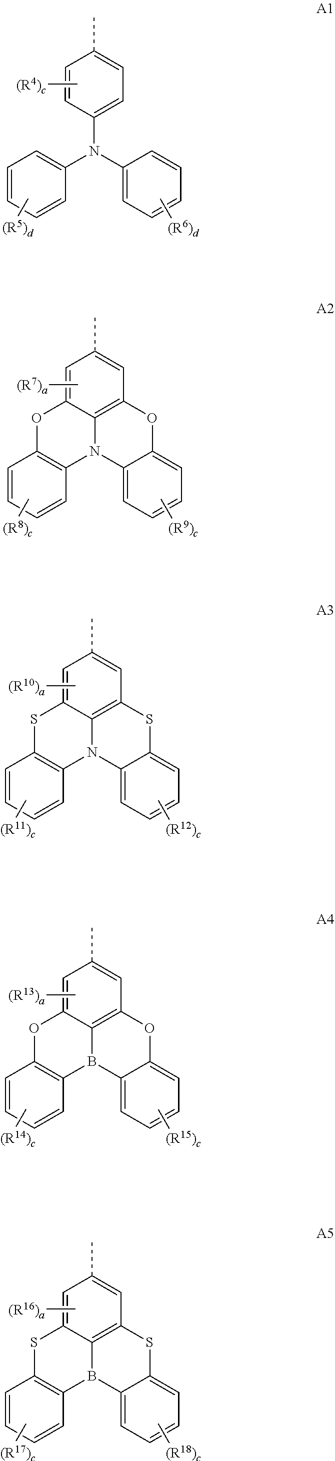 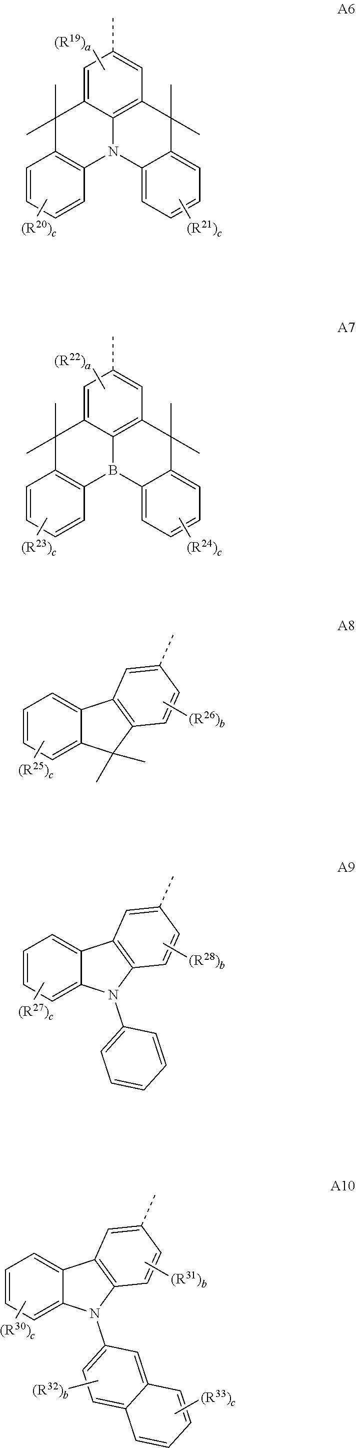 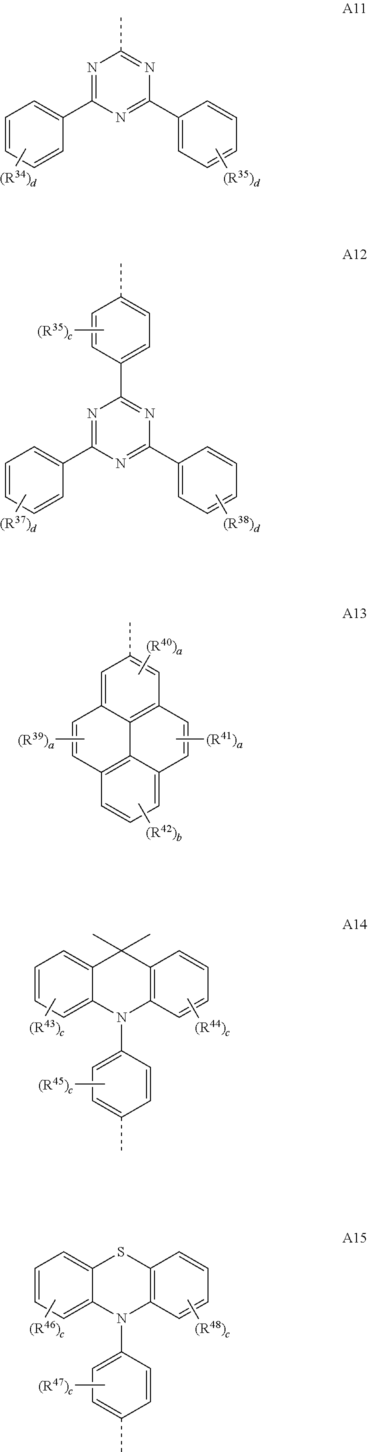 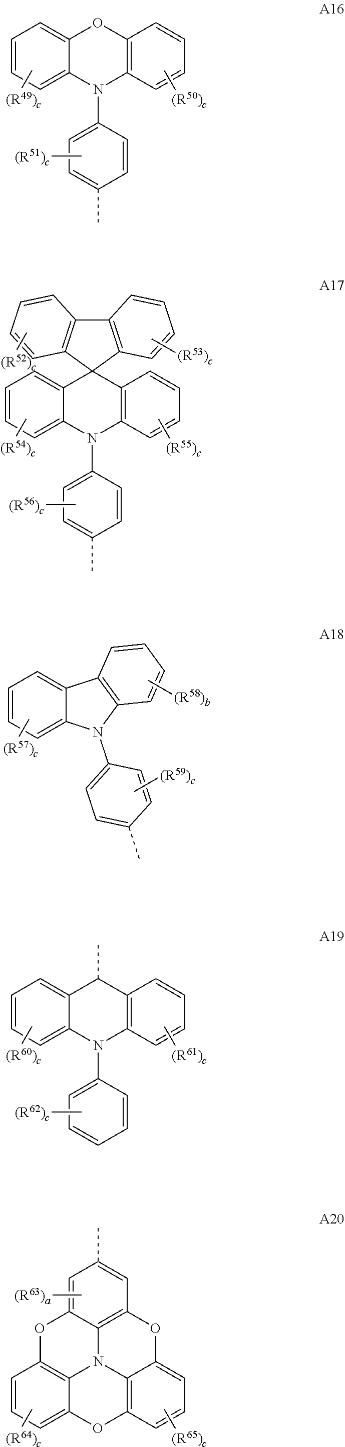
wherein:
a is an integer from 0 to 2; b is an integer from 0 to 3; c is an integer from 0 to 4; d is an integer from 0 to 5;
R⁴-R⁶⁵ are each independently selected from F, Cl, Br, I, D, CN, NO₂, CF₃, B(OR₀)₂, Si(R₀)₃, a linear alkane, an alkane ether, a C₁-C₁₀ alkane sulfide, a C₁-C₁₀ branched alkane, a C₁-C₁₀ cycloalkane, a C₃-C₁₀ alkane ether, a C₃-C₁₀ alkane sulfide group, or a C₆-C₁₀ aryl group;
each dotted line independently represents a bond directly linked to an azacarbene carbon free radical group.

16. The azacarbene carbon free radical molecule according to claim 8, wherein each Ar¹ is independently selected from one of formulas A1-A20: 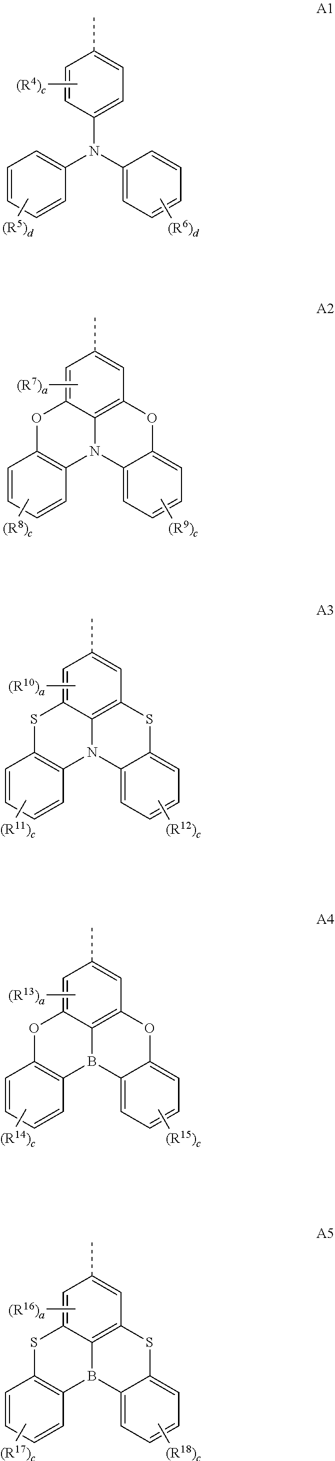 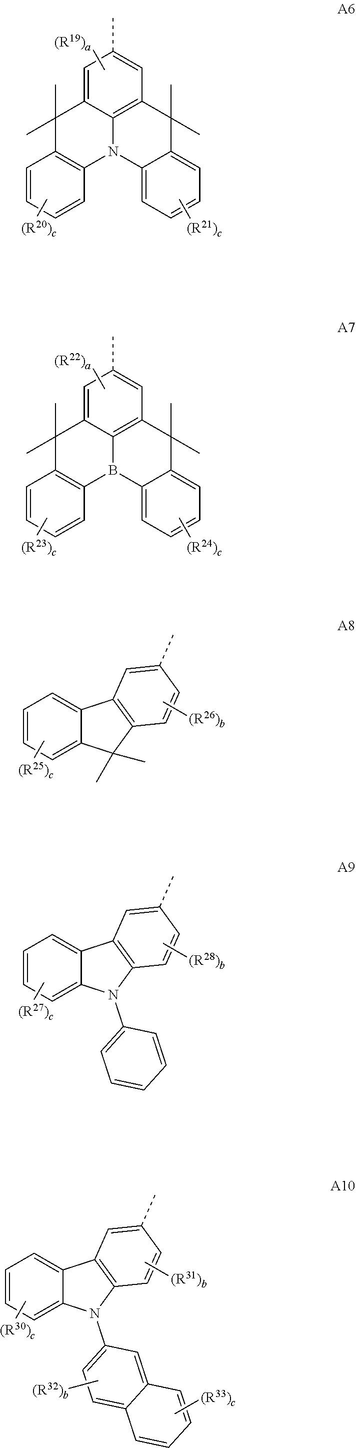 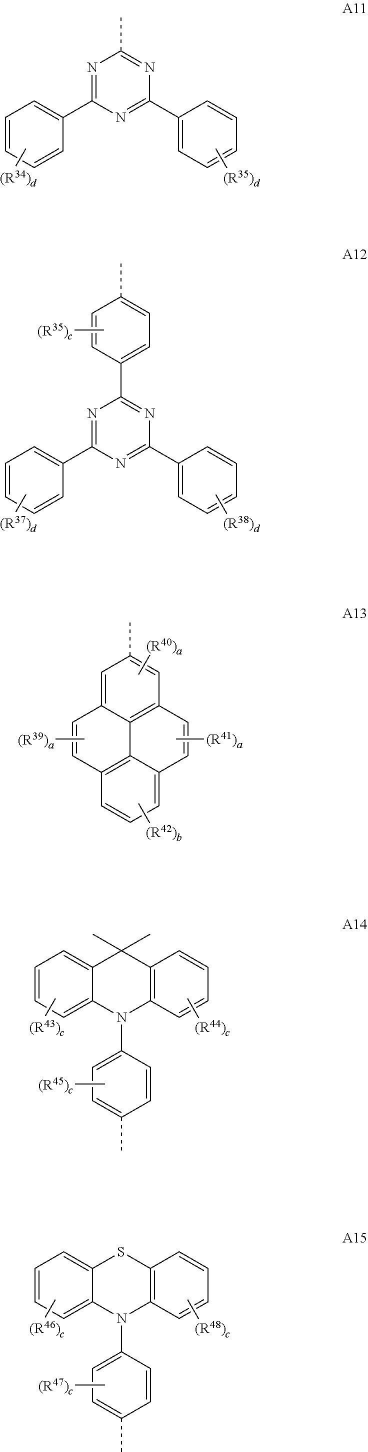 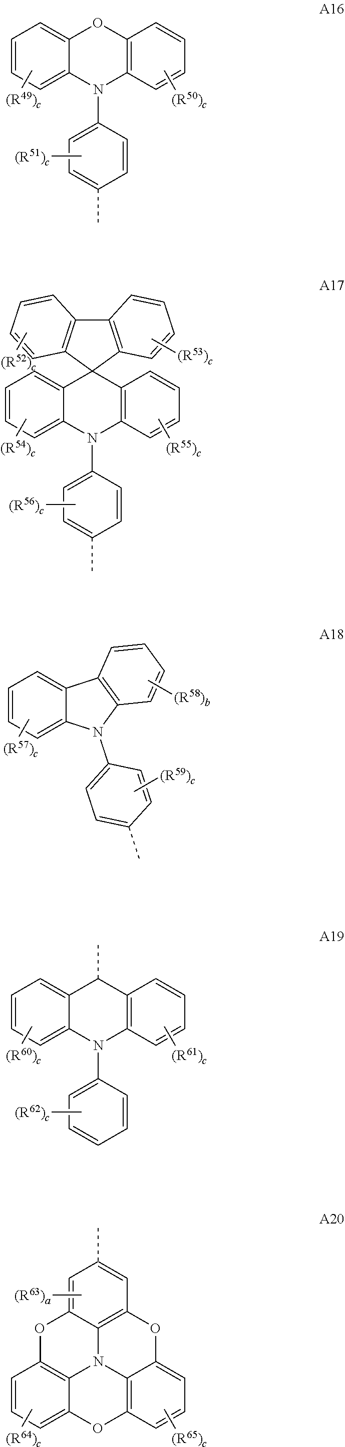
wherein:
a is an integer from 0 to 2; b is an integer from 0 to 3; c is an integer from 0 to 4; d is an integer from 0 to 5;
R⁴-R⁶⁵ are each independently selected from F, Cl, Br, I, D, CN, NO₂, CF₃, B(OR₀)₂, Si(R₀)₃, a linear alkane, an alkane ether, a C₁-C₁₀ alkane sulfide, a C₁-C₁₀ branched alkane, a C₁-C₁₀ cycloalkane, a C₃-C₁₀ alkane ether, a C₃-C₁₀ alkane sulfide group, or a C₆-C₁₀ aryl group;
each dotted line independently represents a bond directly linked to an azacarbene carbon free radical group.

17. A formulation, comprising at least one of the azacarbene carbon free radical molecule according to claim 1, and at least one organic solvent.

18. An organic electronic device, comprising at least one of the azacarbene carbon free radical molecule according to claim 1.

19. The organic electronic device according to claim 18, wherein the organic electronic device is selected from an organic light-emitting diode, an organic photovoltaic cell, an organic light emitting electrochemical cell, an organic field effect transistor, an organic light emitting field effect transistor, an organic laser, an organic spintronic device, an organic spin-valve, a photodiode, an organic sensor, or an organic plasmon emitting diode.
